(19) United States
(12) Patent Application Publication
(10) Pub. No.: US 2019/0062428 A1
(43) Pub. Date: Feb. 28, 2019

(54) COMBINATION OF ANTI-CD47 ANTIBODIES AND CELL DEATH-INDUCING AGENTS, AND USES THEREOF

(71) Applicant: Surface Oncology, Inc., Cambridge, MA (US)

(72) Inventors: Alison PATERSON, Dedham, MA (US); Marisa O. PELUSO, Natick, MA (US); Vito PALOMBELLA, Needham, MA (US); Ammar E. ADAM, Acton, MA (US)

(21) Appl. No.: 16/012,538

(22) Filed: Jun. 19, 2018

Related U.S. Application Data
(60) Provisional application No. 62/521,644, filed on Jun. 19, 2017.

(51) Int. Cl. C07K 16/28; A61P 35/02; A61P 35/00; A61K 39/395; A61K 31/635; A61K 45/06; A61K 31/495

(52) U.S. Cl. CPC C07K 16/2803; A61P 35/02; A61P 35/00; A61K 39/3955; A61K 2039/572; A61K 45/06; A61K 31/495; C07K 2317/565; C07K 2317/56; A61K 31/635

(57) ABSTRACT

The present disclosure relates to combinations of anti-CD47 antibodies and cell death-inducing agents. The disclosure also relates to methods for treating or ameliorating one or more symptoms of a disease, such as cancer, by administering the combination.

### RELATED APPLICATION

This application claims the benefit of U.S. Provisional Application Ser. No. 62/521,644, filed on Jun. 19, 2017. The entire contents of the above-referenced application is incorporated herein by this reference.

### BACKGROUND

The transmembrane protein CD47, also known as integrin-associated protein (TAP), ovarian cancer antigen OA3, Rh-related antigen and MER6, is an immunoglobulin superfamily member involved in multiple cellular processes, including migration, adhesion and T cell function. CD47 was originally identified as a tumor antigen on human ovarian cancer and was subsequently shown to be expressed on multiple human tumor types, including both hematologic and solid tumors. The interaction between CD47 and regulatory protein alpha (SIRPα), an inhibitory protein expressed on macrophages, prevents phagocytosis of CD47-expressing cells. CD47 is expressed at low levels on virtually all non-malignant cells, and loss of expression or changes in membrane distribution can serve as markers of aged or damaged cells, particularly on red blood cells (RBC).

However, high expression of CD47 on cancer cells blocks phagocytic uptake, subsequent antigen cross-presentation and T cell activation, which collectively contribute to tumor immune evasion. Certain human leukemias upregulate CD47 to evade macrophage killing (U.S. Pat. No. 8,562,997). In many hematologic cancers, high CD47 expression is believed to be associated with poor clinical outcomes, for example, Non-Hodgkin Lymphoma, Acute Lymphocytic Leukemia, etc. (U.S. Pat. No. 9,045,541). Similarly, high CD47 expression has been observed in solid tumors such as small cell lung cancer (see, Weiskopf et al, (2016) J. Clin. Investigation 124(7): 2610-2620). Agents that block CD47-SIRPα interaction can restore phagocytic uptake of CD47+ target cells and lower the threshold for macrophage activation, thereby leading to cancer cell destruction.

Although several agents targeting the CD47-SIRPα interaction have been developed, their combinatorial use with other standard-of-care or novel cancer therapies is an area of innovation. Combinatorial therapy has become an important development in cancer treatment, however, determining which therapies are more effective when combined is not intuitive. New combination therapies are needed to more effectively combat various cancers.

### SUMMARY OF THE DISCLOSURE

The present disclosure is based, at least in part, on the discovery that treatment with a combination of a monoclonal antibody that specifically binds human CD47 and a cell death-inducing agent (e.g., a BCL-2 inhibitor) results in a synergistic effect and inhibits tumor re-growth of a hematological cancer. Moreover, the antibody that specifically binds human CD47 and cell death-inducing agent effect non-overlapping mechanisms of tumor cell destruction (e.g., phagocytosis and apoptosis, respectively, and/or caspase-independent and -dependent pathways of tumor cell death induction, respectively), and thus the combinations described herein are useful for treating cancer.

Accordingly, in some aspects, the disclosure provides a method for treating or delaying progression of a cancer and/or reducing or inhibiting tumor growth in a subject in need thereof, the method comprising administering to the subject an effective amount of a monoclonal antibody that specifically binds human CD47, or antigen binding fragment thereof, and an effective amount of a cell death-inducing agent. In other aspects, the disclosure provides a method of inducing apoptosis of tumor cells in a subject in need thereof, the method comprising administering to the subject an effective amount of monoclonal antibody that specifically binds human CD47, or antigen binding fragment thereof, and an effective amount of a cell death-inducing agent. In some aspects, the cell death-inducing agent is selected from an agent that induces apoptosis, an agent that induces immunogenic cell death (ICD), and an agent that inhibits a DNA damage response pathway.

In related aspects, the disclosure provides a composition comprising a monoclonal antibody that specifically binds human CD47, or an antigen binding fragment thereof, and an optional pharmaceutically acceptable carrier, for use in treating or delaying progression of cancer in a subject, wherein the treatment comprises administration of the monoclonal antibody, or antigen binding fragment thereof, in combination with a second composition, wherein the second composition comprises a cell-death inducing agent, and an optional pharmaceutically acceptable carrier. In some aspects, the cell death-inducing agent is selected from an agent that induces apoptosis, an agent that induces immunogenic cell death (ICD), and an agent that inhibits a DNA damage response pathway.

In any of the foregoing or related aspects of the disclosure, the cell death-inducing agent is an agent that induces apoptosis of tumor cells. In some aspects, the cell death-inducing agent is selected from an agent that induces apoptosis, an agent that inhibits a DNA damage response pathway, or a proteasome inhibitor. In some aspects, the cell death-inducing agent is an agent that induces apoptosis selected from the group consisting of: an inhibitor of BCL-2, an inhibitor of MCL-1, an inhibitor of BCL-XL, and an inhibitor of MDM2. In some aspects, the inhibitor of BCL-2 is Venetoclax™, Navitoclax, or obatoclax. In some aspects, the MCL-1 inhibitor is AMG176. In some aspects, the BCL-XL inhibitor is WEHI-539. In some aspects, the MDM2 inhibitor is AMG232.

In any of the foregoing and related aspects of the disclosure, the cell death-inducing agent is an agent that inhibits a DNA damage response pathway selected from an inhibitor of poly ADP ribose polymerase (PARP). In some aspects, the inhibitor of PARP is Olapirib, Niraparib or Rucaparib. In some aspects, the cell death-inducing agent is an agent that inhibits a DNA damage response pathway selected from temozolomide.

In any of the foregoing and related aspects of the disclosure, the cell death-inducing agent is a proteasome inhibitor, wherein the proteasome inhibitor is bortezomib or ixazomib.

In any of the foregoing or related aspects of the disclosure, the cell death-inducing agent is a cytotoxic chemotherapeutic agent, e.g., a chemotherapeutic agent that induces immunogenic cell death. In some aspects, the cytotoxic chemotherapeutic agent is selected from the group consisting of: anthracyclines, topoisomerase inhibitors, bleomycin, gemcitabine, platins, taxanes, DNA alkylating agents, CHOP and fluorouracil.

In any of the foregoing or related aspects of the disclosure, the cell death-inducing agent (e.g., an agent that induces apoptosis, an agent that induces immunogenic cell death (ICD), an agent that inhibits a DNA damage response pathway or a cytotoxic chemotherapeutic agent) enhances surface expression of CD47 on tumor cells, enhances release of soluble CD47, enhances release of membrane-bound fragments or exosomes comprising CD47, and or any combination thereof. In some aspects the cell death-inducing agent is an inhibitor of BCL-2 (e.g., Venetoclax™).

In any of the foregoing or related aspects of the disclosure, the cell death-inducing agent (e.g., an agent that induces apoptosis, an agent that induces immunogenic cell death (ICD), an agent that inhibits a DNA damage response pathway or a cytotoxic chemotherapeutic agent) enhances expression of at least one signal that induces phagocytosis, e.g., calreticulin. In some aspects the cell death-inducing agent is an inhibitor of BCL-2 (e.g., Venetoclax™).

In any of the foregoing or related aspects of the disclosure, the monoclonal antibody that specifically binds human CD47, or antigen binding fragment thereof, binds to human CD47 expressed on tumor cells. In some aspects, the monoclonal antibody that specifically binds human CD47, or antigen binding fragment thereof, blocks the interaction between CD47 and SIRPα. In some aspects, blocking the interaction between CD47 and SIRPα induces macrophage phagocytosis of tumor cells expressing CD47. In some aspects, the monoclonal antibody that specifically binds human CD47, or antigen binding fragment thereof, does not cause significant hemagglutination of human erythrocytes.

In other aspects, the disclosure provides a method for treating or delaying progression of a cancer, reducing or inhibiting tumor growth, and/or inducing apoptosis of tumor cells in a subject in need thereof, the method comprising administering to the subject an effective amount of a monoclonal antibody that specifically binds human CD47, or antigen binding fragment thereof, and an effective amount of a cell death-inducing agent (e.g., an agent that induces apoptosis, an agent that induces immunogenic cell death (ICD), an agent that inhibits a DNA damage response pathway or a cytotoxic chemotherapeutic agent). In some aspects the cell death-inducing agent is an inhibitor of BCL-2 (e.g., Venetoclax™).

In some aspects, the method or composition of the disclosure features a combination of an inhibitor of BCL-2 (e.g., Venetoclax™) and a monoclonal antibody that specifically binds human CD47, or antigen binding fragment thereof, wherein the monoclonal antibody comprises:

a heavy chain complementarity determining region 1 (HC CDR1) comprising the amino acid sequence set forth in SEQ ID NO: 5;

a heavy chain complementarity determining region 2 (HC CDR2) comprising the amino acid sequence set forth in SEQ ID NO: 6;

a heavy chain complementarity determining region 3 (HC CDR3) comprising the amino acid sequence set forth in SEQ ID NO: 7;

a light chain complementarity determining region 1 (LC CDR1) comprising the amino acid sequence set forth in SEQ ID NO: 8;

a light chain complementarity determining region 2 (LC CDR2) comprising the amino acid sequence set forth in SEQ ID NO: 9; and

a light chain complementarity determining region 3 (LC CDR3) comprising the amino acid sequence set forth in SEQ ID NO: 10.

In some aspects, the method or composition of the disclosure features a combination of a cell death-inducing agent (e.g., an agent that induces apoptosis, an agent that induces immunogenic cell death (ICD), an agent that inhibits a DNA damage response pathway or a cytotoxic chemotherapeutic agent), such as an inhibitor of BCL-2 (e.g., Venetoclax™) and a monoclonal antibody that specifically binds to human CD47, or antigen binding fragment thereof, wherein the monoclonal antibody comprises a heavy chain variable region (VH) comprising the amino acid sequence set forth in SEQ ID NO: 3 and a light chain variable region (VL) comprising the amino acid sequence set forth in SEQ ID NO: 4. In some aspects, the monoclonal antibody that specifically binds human CD47, or antigen binding fragment thereof, is a human antibody.

In any of the foregoing or related aspects of the disclosure, the monoclonal antibody that specifically binds human CD47, or antigen binding fragment thereof, comprises a wild-type human IgG1 or a wild-type human IgG4 heavy chain constant region. In some aspects, the monoclonal antibody that specifically binds human CD47, or antigen binding fragment thereof, comprises a mutant human IgG1 or a mutant human IgG4 heavy chain constant region. In some aspects, the mutant human IgG1 heavy chain constant region comprises a substitution at Glu233, Leu234, Leu235, Asn297, or a combination thereof, numbering according to EU numbering. In some aspects, the mutant IgG1 heavy chain constant region comprises an E233P substitution, an L234A or L234E substitution, an L235A substitution, an N297A substitution, or a combination thereof, numbering according to EU numbering. In some aspects, the mutant IgG4 heavy chain constant region comprises a substitution at Ser228, Leu235, Asn297, or a combination thereof, numbering according to EU numbering. In some aspects, the mutant IgG4 heavy chain constant region comprises an S228P substitution, an L235E substitution, an N297A substitution, or a combination thereof, numbering according to EU numbering. In some aspects, the mutant IgG4 heavy chain constant region comprises an S228P substitution and an L235E substitution, numbering according to EU numbering. In some aspects, the monoclonal antibody that specifically binds human CD47, or antigen binding fragment thereof, comprises a human kappa constant region. In some aspects, the wild-type human IgG4 heavy chain constant region comprises the amino acid sequence set forth in SEQ ID NO: 21. In some aspects, the human kappa constant region comprises the amino acid sequence set forth in SEQ ID NO: 17.

In some aspects, the method or composition of the disclosure features a combination of a cell death-inducing agent (e.g., an agent that induces apoptosis, an agent that induces immunogenic cell death (ICD), an agent that inhibits a DNA damage response pathway or a cytotoxic chemotherapeutic agent), such as an inhibitor of BCL-2 (e.g., Venetoclax™) and a monoclonal antibody that specifically binds human CD47, or antigen binding fragment thereof, wherein the monoclonal antibody comprises a heavy chain comprising the amino acid sequence set forth in SEQ ID NO: 11, SEQ ID NO: 12, SEQ ID NO: 13, or SEQ ID NO: 14; and a light chain comprising the amino acid sequence set forth in SEQ ID NO: 15 or SEQ ID NO: 16. In some aspects, the monoclonal antibody that specifically binds human CD47, or antigen binding fragment thereof, comprises a heavy chain comprising the amino acid sequence set forth in SEQ ID NO: 13; and a light chain comprising the amino acid sequence set forth in SEQ ID NO: 16.

In any of the foregoing or related aspects of the disclosure, the monoclonal antibody or antigen binding fragment is administered preceding or subsequent to administration of the cell-death inducing agent. In some aspects, the cell-death inducing agent is administered preceding or subsequent to administration of the monoclonal antibody of antigen binding fragment. In some aspects the monoclonal antibody or antigen-binding fragment is administered intravenously. In some aspects, the cell death-inducing agent (e.g., an agent that induces apoptosis, an agent that induces immunogenic cell death (ICD), and an agent that inhibits a DNA damage response pathway or a cytotoxic chemotherapeutic agent), such as an inhibitor of BCL-2 (e.g., Venetoclax™) is administered orally.

In any of the foregoing or related aspects of the disclosure, the cancer or tumor is a hematological cancer or hematological tumor. In some aspects, the hematological cancer or tumor is chronic lymphocytic leukemia (CLL), acute lymphocytic leukemia (ALL), chronic myelogenous leukemia (CML), diffuse large cell B cell lymphoma (DLBCL), follicular lymphoma (FL), Non-Hodgkin's lymphoma, myelofibrosis, mastocytosis, mantle cell lymphoma, multiple myeloma (MM) or acute myeloid leukemia (AML). In some aspects, the cancer or tumor is a cancer or tumor of a tissue selected from the group consisting of lung, pancreas, breast, liver, ovary, testicle, kidney, bladder, spine, brain, cervix, endrometrium, colon/rectum, anus, esophagus, gallbladder, gastrointestinal tract, skin, prostate, testicle, pituitary, stomach, uterus, vagina and thyroid.

Other aspects of the disclosure relate to use of a composition comprising a monoclonal antibody that specifically binds human CD47, or antigen-binding fragment thereof, as disclosed herein, and an optional pharmaceutically acceptable carrier, in the manufacture of a medicament for treating or delaying progression of cancer, or reducing or inhibiting tumor growth, and/or inducing apoptosis of tumor cells in a subject, wherein the medicament comprises the composition and an optional pharmaceutically acceptable carrier, and wherein the treatment comprises administration of the medicament in combination with a second composition, wherein the second composition comprises a cell-death inducing agent (e.g., an agent that induces apoptosis, an agent that induces immunogenic cell death (ICD), an agent that inhibits a DNA damage response pathway or a cytotoxic chemotherapeutic agent), such as an inhibitor of BCL-2 (e.g., Venetoclax™), and an optional pharmaceutically acceptable carrier.

In other aspects, the disclosure provides a kit comprising a medicament comprising a composition comprising a monoclonal antibody that specifically binds human CD47, or antigen-binding fragment thereof, as disclosed herein, and an optional pharmaceutically acceptable carrier, and a package insert comprising instructions for administration of the medicament in combination with a second medicament comprising a composition comprising a cell-death inducing agent (e.g., an agent that induces apoptosis, an agent that induces immunogenic cell death (ICD), an agent that inhibits a DNA damage response or a cytotoxic chemotherapeutic agent), such as an inhibitor of BCL-2 (e.g., Venetoclax™), and an optional pharmaceutically acceptable carrier, for treating or delaying progression of cancer in a subject.

In some aspects, the kit comprises a container comprising a composition comprising a monoclonal antibody that specifically binds human CD47, or antigen-binding fragment thereof, as disclosed herein, and an optional pharmaceutically acceptable carrier, and a package insert comprising instructions for administration of the composition in combination with a second composition comprising a cell-death inducing agent (e.g., an agent that induces apoptosis, an agent that induces immunogenic cell death (ICD), an agent that inhibits a DNA damage response or a cytotoxic chemotherapeutic agent), such as an inhibitor of BCL-2 (e.g., Venetoclax™), and an optional pharmaceutically acceptable carrier, for treating or delaying progression of cancer in a subject.

In some aspects, the kit comprises a container comprising composition comprising a monoclonal antibody that specifically binds human CD47, or antigen binding fragment thereof, wherein the monoclonal antibody comprises:

a heavy chain complementarity determining region 1 (HC CDR1) comprising the amino acid sequence set forth in SEQ ID NO: 5;

a heavy chain complementarity determining region 2 (HC CDR2) comprising the amino acid sequence set forth in SEQ ID NO: 6;

a heavy chain complementarity determining region 3 (HC CDR3) comprising the amino acid sequence set forth in SEQ ID NO: 7;

a light chain complementarity determining region 1 (LC CDR1) comprising the amino acid sequence set forth in SEQ ID NO: 8;

a light chain complementarity determining region 2 (LC CDR2) comprising the amino acid sequence set forth in SEQ ID NO: 9; and

a light chain complementarity determining region 3 (LC CDR3) comprising the amino acid sequence set forth in SEQ ID NO: 10, and an optional pharmaceutically acceptable carrier, and a package insert comprising instructions for administration of the composition in combination with a second composition comprising an inhibitor of BCL-2 (e.g., Venetoclax™), and an optional pharmaceutically acceptable carrier.

In some aspects, the kit comprises a container comprising composition comprising a monoclonal antibody that specifically binds human CD47, or antigen binding fragment thereof, wherein the monoclonal antibody comprises a heavy chain variable region (VH) comprising the amino acid sequence set forth in SEQ ID NO: 3 and a light chain variable region (VL) comprising the amino acid sequence set forth in SEQ ID NO: 4, and an optional pharmaceutically acceptable carrier, and a package insert comprising instructions for administration of the composition in combination with a second composition comprising an inhibitor of BCL-2 (e.g., Venetoclax™), and an optional pharmaceutically acceptable carrier.

In some aspects, the monoclonal antibody that specifically binds human CD47, or antigen binding fragment thereof, is a human antibody. In any of the foregoing or related aspects of the disclosure, the monoclonal antibody that specifically binds human CD47, or antigen binding fragment thereof, comprises a wild-type human IgG1 or a wild-type human IgG4 heavy chain constant region. In some aspects, the monoclonal antibody that specifically binds human CD47, or antigen binding fragment thereof, comprises a mutant human IgG1 or a mutant human IgG4 heavy chain constant region. In some aspects, the mutant human IgG1 heavy chain constant region comprises a substitution at Glu233, Leu234, Leu235, Asn297, or a combination thereof, numbering according to EU numbering. In some aspects, the mutant IgG1 heavy chain constant region comprises an E233P substitution, an L234A or L234E substitution, an L235A substitution, an N297A substitution, or a combination thereof, numbering according to EU numbering. In some aspects, the mutant IgG4 heavy chain constant region comprises a substitution at Ser228, Leu235, Asn297, or a combination thereof, numbering according to EU numbering. In some aspects, the mutant IgG4 heavy chain constant region comprises an S228P substitution, an L235E substitution, an N297A substitution, or a combination thereof, numbering according to EU numbering. In some aspects, the mutant IgG4 heavy chain constant region comprises an S228P substitution and an L235E substitution, numbering according to EU numbering. In some aspects, the monoclonal antibody that specifically binds human CD47, or antigen binding fragment thereof, comprises a human kappa constant region. In some aspects, the wild-type human IgG4 heavy chain constant region comprises the amino acid sequence set forth in SEQ ID NO: 21. In some aspects, the human kappa constant region comprises the amino acid sequence set forth in SEQ ID NO: 17.

In some aspects, the kit comprises a container comprising composition comprising a monoclonal antibody that specifically binds human CD47, or antigen binding fragment thereof, wherein the monoclonal antibody comprises a heavy chain comprising the amino acid sequence set forth in SEQ ID NO: 11, SEQ ID NO: 12, SEQ ID NO: 13, or SEQ ID NO: 14; and a light chain comprising the amino acid sequence set forth in SEQ ID NO: 15 or SEQ ID NO: 16, and an optional pharmaceutically acceptable carrier, and a package insert comprising instructions for administration of the composition in combination with a second composition comprising an inhibitor of BCL-2 (e.g., Venetoclax™), and an optional pharmaceutically acceptable carrier. In some aspects, the monoclonal antibody that specifically binds human CD47, or antigen binding fragment thereof, comprises a heavy chain comprising the amino acid sequence set forth in SEQ ID NO: 13; and a light chain comprising the amino acid sequence set forth in SEQ ID NO: 16.

In some aspects, the kit includes instructions for administration of the composition comprising the monoclonal antibody or antigen binding fragment preceding or subsequent to administration of the second composition comprising a cell-death inducing agent. In some aspects, the kit includes instructions for administration of the second composition comprising a cell-death inducing agent preceding or subsequent to administration of the composition comprising the monoclonal antibody of antigen binding fragment. In some aspects the kit includes instructions for administration of the composition comprising the monoclonal antibody or antigen-binding fragment intravenously. In some aspects, the kit includes instructions for administration of the composition comprising the cell death-inducing agent (e.g., an agent that induces apoptosis, an agent that induces immunogenic cell death (ICD), an agent that inhibits a DNA damage response or a cytotoxic chemotherapeutic agent), such as an inhibitor of BCL-2 (e.g., Venetoclax™) orally.

In some aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof, and an effective amount an inhibitor of BCL-2, wherein the monoclonal antibody or antigen binding fragment comprises:

a heavy chain complementarity determining region 1 (HC CDR1) comprising the amino acid sequence set forth in SEQ ID NO: 5;

a heavy chain complementarity determining region 2 (HC CDR2) comprising the amino acid sequence set forth in SEQ ID NO: 6;

a heavy chain complementarity determining region 3 (HC CDR3) comprising the amino acid sequence set forth in SEQ ID NO: 7;

a light chain complementarity determining region 1 (LC CDR1) comprising the amino acid sequence set forth in SEQ ID NO: 8;

a light chain complementarity determining region 2 (LC CDR2) comprising the amino acid sequence set forth in SEQ ID NO: 9; and

a light chain complementarity determining region 3 (LC CDR3) comprising the amino acid sequence set forth in SEQ ID NO: 10.

In some aspects, the inhibitor of BCL-2 is selected from Venetoclax™, Navitoclax, or obatoclax. In some aspects, the inhibitor of BCL-2 is Venetoclax™.

In other aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof, and an effective amount an inhibitor of BCL-2, wherein the inhibitor of BCL-2 is selected from Venetoclax™, Navitoclax, or obatoclax, or is Venetoclax™ and the monoclonal antibody that specifically binds to human CD47, or antigen binding fragment thereof, comprises a heavy chain variable region (VH) comprising the amino acid sequence set forth in SEQ ID NO: 3 and a light chain variable region (VL) comprising the amino acid sequence set forth in SEQ ID NO: 4. In some aspects, the monoclonal antibody comprises a wild-type human IgG4 heavy chain constant region. In some aspects, the wild-type human IgG4 heavy chain constant region comprises the amino acid sequence set forth in SEQ ID NO: 21. In some aspects, the monoclonal antibody comprises a human kappa constant region. In some aspects, the human kappa constant region comprises the amino acid sequence set forth in SEQ ID NO: 17.

In other aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof, and an effective amount an inhibitor of BCL-2, wherein the inhibitor of BCL-2 is selected from Venetoclax™, Navitoclax, or obatoclax, or is Venetoclax™ and the monoclonal antibody that specifically binds to human CD47 comprises a heavy chain comprising the amino acid sequence set forth in SEQ ID NO: 13; and a light chain comprising the amino acid sequence set forth in SEQ ID NO: 16.

In other aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof (e.g., comprises the VH and VL regions set forth in SEQ ID NOs 3 and 4 or comprises the heavy and light chains set forth in SEQ ID NOs 13 and 16), an effective amount an inhibitor of BCL-2, and an effective amount of an MCL-1 inhibitor. In some aspects, the inhibitor of BCL-2 is selected from Venetoclax™, Navitoclax, or obatoclax. In some aspects, the inhibitor of BCL-2 is Venetoclax™. In some aspects, the cancer or tumor is sensitive to Venetoclax™ treatment. In some aspects, the cancer or tumor is resistant to Venetoclax™ treatment.

In some aspects, the subject is administered a combination therapy comprising an anti-CD47 antibody as described herein, an inhibitor of BCL-2 (e.g., Venetoclax™), optionally an MCL-1 inhibitor, and an effective amount of a hypomethylating agent. In some aspects, the hypomethylating agent is selected from azacitidine, and decitibine. In some aspects, the hypomethylating agent is azacitidine.

In other aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof, and an effective amount an inhibitor of MCL-1, wherein the monoclonal antibody that specifically binds to human CD47, or antigen binding fragment thereof, comprises a heavy chain variable region (VH) comprising the amino acid sequence set forth in SEQ ID NO: 3 and a light chain variable region (VL) comprising the amino acid sequence set forth in SEQ ID NO: 4. In some aspects, the monoclonal antibody comprises a wild-type human IgG4 heavy chain constant region. In some aspects, the wild-type human IgG4 heavy chain constant region comprises the amino acid sequence set forth in SEQ ID NO: 21. In some aspects, the monoclonal antibody comprises a human kappa constant region. In some aspects, the human kappa constant region comprises the amino acid sequence set forth in SEQ ID NO: 17.

In other aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof, and an effective amount an inhibitor of MCL-1, wherein the monoclonal antibody that specifically binds to human CD47 comprises a heavy chain comprising the amino acid sequence set forth in SEQ ID NO: 13; and a light chain comprising the amino acid sequence set forth in SEQ ID NO: 16.

In other aspects, the disclosure provides methods and compositions for treating or delaying progression of a cancer or reducing or inhibiting tumor growth in a subject in need thereof, the method comprising administering to the subject an effective amount of a monoclonal antibody that specifically binds human CD47, or antigen binding fragment thereof, and an effective amount an anthracycline, wherein the monoclonal antibody or antigen binding fragment comprises:

a heavy chain complementarity determining region 1 (HC CDR1) comprising the amino acid sequence set forth in SEQ ID NO: 5;

a heavy chain complementarity determining region 2 (HC CDR2) comprising the amino acid sequence set forth in SEQ ID NO: 6;

a heavy chain complementarity determining region 3 (HC CDR3) comprising the amino acid sequence set forth in SEQ ID NO: 7;

a light chain complementarity determining region 1 (LC CDR1) comprising the amino acid sequence set forth in SEQ ID NO: 8;

a light chain complementarity determining region 2 (LC CDR2) comprising the amino acid sequence set forth in SEQ ID NO: 9; and

a light chain complementarity determining region 3 (LC CDR3) comprising the amino acid sequence set forth in SEQ ID NO: 10.

In some aspects, the anthracycline is selected from doxorubicin, daunorubicin, epirubicin, idarubicin, and valrubicin. In some aspects, the anthracycline is doxorubicin.

In other aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof, and an effective amount an anthracycline, wherein the anthracycline is selected from doxorubicin, daunorubicin, epirubicin, idarubicin, and valrubicin, or is doxorubicin, and the monoclonal antibody that specifically binds to human CD47, or antigen binding fragment thereof, comprises a heavy chain variable region (VH) comprising the amino acid sequence set forth in SEQ ID NO: 3 and a light chain variable region (VL) comprising the amino acid sequence set forth in SEQ ID NO: 4. In some aspects, the monoclonal antibody comprises a wild-type human IgG4 heavy chain constant region. In some aspects, the wild-type human IgG4 heavy chain constant region comprises the amino acid sequence set forth in SEQ ID NO: 21. In some aspects, the monoclonal antibody comprises a human kappa constant region. In some aspects, the human kappa constant region comprises the amino acid sequence set forth in SEQ ID NO: 17.

In other aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof, and an effective amount an anthracycline, wherein the anthracycline is selected from doxorubicin, daunorubicin, epirubicin, idarubicin, and valrubicin, or is doxorubicin, and the monoclonal antibody that specifically binds to human CD47 comprises a heavy chain comprising the amino acid sequence set forth in SEQ ID NO: 13; and a light chain comprising the amino acid sequence set forth in SEQ ID NO: 16.

In some aspects, the subject is administered a combination therapy comprising an anti-CD47 antibody as described herein, an anthracycline (e.g., doxorubicin), and an effective amount of a hypomethylating agent. In some aspects, the hypomethylating agent is selected from azacitidine, and decitibine. In some aspects, the hypomethylating agent is azacitidine.

In other aspects, the disclosure provides methods and compositions for treating or delaying progression of a cancer or reducing or inhibiting tumor growth in a subject in need thereof, the method comprising administering to the subject an effective amount of a monoclonal antibody that specifically binds human CD47, or antigen binding fragment thereof, and an effective amount a proteasome inhibitor, wherein the monoclonal antibody or antigen binding fragment comprises:

a heavy chain complementarity determining region 1 (HC CDR1) comprising the amino acid sequence set forth in SEQ ID NO: 5;

a heavy chain complementarity determining region 2 (HC CDR2) comprising the amino acid sequence set forth in SEQ ID NO: 6;

a heavy chain complementarity determining region 3 (HC CDR3) comprising the amino acid sequence set forth in SEQ ID NO: 7;

a light chain complementarity determining region 1 (LC CDR1) comprising the amino acid sequence set forth in SEQ ID NO: 8;

a light chain complementarity determining region 2 (LC CDR2) comprising the amino acid sequence set forth in SEQ ID NO: 9; and

a light chain complementarity determining region 3 (LC CDR3) comprising the amino acid sequence set forth in SEQ ID NO: 10.

In some aspects, the proteasome inhibitor is selected from bortezomib, carfilzomib, and ixazomib. In some aspects, the proteasome inhibitor is bortezomib.

In other aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof, and an effective amount of a proteasome inhibitor, wherein the proteasome inhibitor is selected from bortezomib, carfilzomib, and ixazomib, or is bortezomib, and the monoclonal antibody that specifically binds to human CD47, or antigen binding fragment thereof, comprises a heavy chain variable region (VH) comprising the amino acid sequence set forth in SEQ ID NO: 3 and a light chain variable region (VL) comprising the amino acid sequence set forth in SEQ ID NO: 4. In some aspects, the monoclonal antibody comprises a wild-type human IgG4 heavy chain constant region. In some aspects, the wild-type human IgG4 heavy chain constant region comprises the amino acid sequence set forth in SEQ ID NO: 21. In some aspects, the monoclonal antibody comprises a human kappa constant region. In some aspects, the human kappa constant region comprises the amino acid sequence set forth in SEQ ID NO: 17.

In other aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof, and an effective amount of a proteasome inhibitor, wherein the proteasome inhibitor is selected from bortezomib, carfilzomib, and ixazomib, or is bortezomib, and the monoclonal antibody that specifically binds to human CD47 comprises a heavy chain comprising the amino acid sequence set forth in SEQ ID NO: 13; and a light chain comprising the amino acid sequence set forth in SEQ ID NO: 16.

In some aspects, the subject is administered a combination therapy comprising an anti-CD47 antibody as described herein, a proteasome inhibitor (e.g., bortezomib), and an effective amount of a hypomethylating agent. In some aspects, the hypomethylating agent is selected from azacitidine, and decitibine. In some aspects, the hypomethylating agent is azacitidine.

In other aspects, the disclosure provides methods and compositions for treating or delaying progression of a cancer or reducing or inhibiting tumor growth in a subject in need thereof, the method comprising administering to the subject an effective amount of a monoclonal antibody that specifically binds human CD47, or antigen binding fragment thereof, and an effective amount a platinum derivative, wherein the monoclonal antibody or antigen binding fragment comprises:

a heavy chain complementarity determining region 1 (HC CDR1) comprising the amino acid sequence set forth in SEQ ID NO: 5;

a heavy chain complementarity determining region 2 (HC CDR2) comprising the amino acid sequence set forth in SEQ ID NO: 6;

a heavy chain complementarity determining region 3 (HC CDR3) comprising the amino acid sequence set forth in SEQ ID NO: 7;

a light chain complementarity determining region 1 (LC CDR1) comprising the amino acid sequence set forth in SEQ ID NO: 8;

a light chain complementarity determining region 2 (LC CDR2) comprising the amino acid sequence set forth in SEQ ID NO: 9; and

a light chain complementarity determining region 3 (LC CDR3) comprising the amino acid sequence set forth in SEQ ID NO: 10.

In some aspects, the platinum derivative is selected from oxaliplatin, carboplatin, and cisplatin. In some aspects, the platinum derivative is oxaliplatin.

In other aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof, and an effective amount of a platinum derivative, wherein the platinum derivative is selected from oxaliplatin, carboplatin, and cisplatin, or is oxaliplatin, and the monoclonal antibody that specifically binds to human CD47, or antigen binding fragment thereof, comprises a heavy chain variable region (VH) comprising the amino acid sequence set forth in SEQ ID NO: 3 and a light chain variable region (VL) comprising the amino acid sequence set forth in SEQ ID NO: 4. In some aspects, the monoclonal antibody comprises a wild-type human IgG4 heavy chain constant region. In some aspects, the wild-type human IgG4 heavy chain constant region comprises the amino acid sequence set forth in SEQ ID NO: 21. In some aspects, the monoclonal antibody comprises a human kappa constant region. In some aspects, the human kappa constant region comprises the amino acid sequence set forth in SEQ ID NO: 17.

In other aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof, and an effective amount of a platinum derivative, wherein the platinum derivative is selected from oxaliplatin, carboplatin, and cisplatin, or is oxaliplatin, and the monoclonal antibody that specifically binds to human CD47 comprises a heavy chain comprising the amino acid sequence set forth in SEQ ID NO: 13; and a light chain comprising the amino acid sequence set forth in SEQ ID NO: 16.

In some aspects, the subject is administered a combination therapy comprising an anti-CD47 antibody as described herein, a platinum derivative (e.g., oxaliplatin), and an effective amount of a hypomethylating agent. In some aspects, the hypomethylating agent is selected from azacitidine, and decitibine. In some aspects, the hypomethylating agent is azacitidine.

In other aspects, the disclosure provides methods and compositions for treating or delaying progression of a cancer or reducing or inhibiting tumor growth in a subject in need thereof, the method comprising administering to the subject an effective amount of a monoclonal antibody that specifically binds human CD47, or antigen binding fragment thereof, and an effective amount an inhibitor of PARP, wherein the monoclonal antibody or antigen binding fragment comprises:

a heavy chain complementarity determining region 1 (HC CDR1) comprising the amino acid sequence set forth in SEQ ID NO: 5;

a heavy chain complementarity determining region 2 (HC CDR2) comprising the amino acid sequence set forth in SEQ ID NO: 6;

a heavy chain complementarity determining region 3 (HC CDR3) comprising the amino acid sequence set forth in SEQ ID NO: 7;

a light chain complementarity determining region 1 (LC CDR1) comprising the amino acid sequence set forth in SEQ ID NO: 8;

a light chain complementarity determining region 2 (LC CDR2) comprising the amino acid sequence set forth in SEQ ID NO: 9; and

a light chain complementarity determining region 3 (LC CDR3) comprising the amino acid sequence set forth in SEQ ID NO: 10.

In some aspects, the inhibitor of PARP is selected from Olaparib, Niraparib and Rucaparib. In some aspects, the inhibitor of PARP is Olaparib.

In other aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof, and an effective amount of an inhibitor of PARP, wherein the inhibitor of PARP is selected from Olaparib, Niraparib and Rucaparib, or is Olaparib, and the monoclonal antibody that specifically binds to human CD47, or antigen binding fragment thereof, comprises a heavy chain variable region (VH) comprising the amino acid sequence set forth in SEQ ID NO: 3 and a light chain variable region (VL) comprising the amino acid sequence set forth in SEQ ID NO: 4. In some aspects, the monoclonal antibody comprises a wild-type human IgG4 heavy chain constant region. In some aspects, the wild-type human IgG4 heavy chain constant region comprises the amino acid sequence set forth in SEQ ID NO: 21. In some aspects, the monoclonal antibody comprises a human kappa constant region. In some aspects, the human kappa constant region comprises the amino acid sequence set forth in SEQ ID NO: 17.

In other aspects, the disclosure provides methods and compositions for treating or delaying progression of a cancer or reducing or inhibiting tumor growth in a subject in need thereof, by administering to the subject an effective amount of a monoclonal antibody that specifically binds human CD47, or antigen binding fragment thereof, and an effective amount of an inhibitor of PARP, wherein the inhibitor of PARP is selected from Olaparib, Niraparib and Rucaparib, or is Olaparib, and the monoclonal antibody that specifically binds to human CD47 comprises a heavy chain comprising the amino acid sequence set forth in SEQ ID NO: 13; and a light chain comprising the amino acid sequence set forth in SEQ ID NO: 16.

In any of the foregoing or related aspects, the subject has a cancer or tumor, wherein the cancer or tumor is selected from chronic lymphocytic leukemia (CLL), acute lymphocytic leukemia (ALL), chronic myelogenous leukemia (CML), diffuse large cell B cell lymphoma (DLBCL), follicular lymphoma (FL), Non-Hodgkin's lymphoma, myelofibrosis, mastocytosis, mantle cell lymphoma, multiple myeloma (MM) or acute myeloid leukemia (AML). In some aspects, the cancer or tumor is a cancer or tumor selected from DLBCL, MM, and AML. In some aspects, the cancer or tumor is DLBCL. In some aspects, the cancer or tumor is MM. In some aspects, the cancer or tumor is AML. In some aspects, the cancer or tumor is ovarian.

In other aspects, the disclosure provides a composition comprising a monoclonal antibody that specifically binds human CD47, or an antigen binding fragment thereof, and an optional pharmaceutically acceptable carrier, for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises a cell-death inducing agent, and an optional pharmaceutically acceptable carrier, wherein the cell death-inducing agent is selected from an agent that induces apoptosis, an agent that induces immunogenic cell death (ICD), and an agent that inhibits a DNA damage response pathway.

In some aspects, the second composition comprises a cell death-inducing agent which is an agent that induces apoptosis selected from the group consisting of: an inhibitor of BCL-2, an inhibitor of MCL-1, an inhibitor of BCL-XL, an inhibitor of MDM2, and a combination thereof. In some aspects, the agent that induces apoptosis is an inhibitor of BCL-2. In some aspects, the inhibitor of BCL-2 is selected from Venetoclax™, Navitoclax, or obatoclax. In some aspects, the second composition comprises Venetoclax™, and an optional pharmaceutical carrier.

In some aspects, the second composition comprises a cell death-inducing agent which is an MCL-1 inhibitor. In some aspects, the MCL-1 inhibitor is selected from AMG176, MIK665 and AZD5991.

In some aspects, the second composition comprises an inhibitor of BCL-2 (e.g., Venetoclax™, Navitoclax, or obatoclax), and an optional pharmaceutical carrier, and the treatment comprises administration of the monoclonal antibody, or antigen binding fragment thereof, in combination with a second and third composition, wherein the third composition comprises an MCL-1 inhibitor. In some aspects, the second composition comprises Venetoclax™, and an optional pharmaceutical carrier. In some aspects, the MCL-1 inhibitor is selected from AMG176, MIK665 and AZD5991.

In some aspects, the second composition comprises an inhibitor of BCL-2 (e.g., Venetoclax™, Navitoclax, or obatoclax) and the treatment comprises administration of the monoclonal antibody, or antigen binding fragment thereof, in combination with a second and third composition, wherein the third composition comprises a hypomethylating agent, and an optional pharmaceutically acceptable carrier. In some aspects, the second composition comprises Venetoclax™, and an optional pharmaceutical carrier. In some aspects, the hypomethylating agent is selected from azacitidine, and decitibine. In some aspects, the third composition comprises azacitidine, and an optional pharmaceutical carrier.

In some aspects, the second composition comprises a cell death-inducing agent which is a BCL-XL inhibitor. In some aspects, the BCL-XL inhibitor is WEHI-539.

In some aspects, the second composition comprises a cell death-inducing agent which is MDM2 inhibitor. In some aspects, the MDM2 inhibitor is AMG232.

In other aspects, the disclosure provides a composition comprising a monoclonal antibody that specifically binds human CD47, or an antigen binding fragment thereof, and an optional pharmaceutically acceptable carrier, for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises a cell-death inducing agent, and an optional pharmaceutically acceptable carrier, wherein the cell death-inducing agent is an agent that induces immunogenic cell death (ICD). In some aspects, the agent that induces ICD is selected from an anthracycline, a proteasome inhibitor, and a platinum derivative.

In some aspects, the second composition comprises an anthracycline, and an optional pharmaceutical carrier. In some aspects, the anthracycline is selected from doxorubicin, daunorubicin, epirubicin, idarubicin, and valrubicin. In some aspects the second composition comprises doxorubicin, and an optional pharmaceutical carrier.

In some aspects, the second composition comprises an anthracycline (e.g., doxorubicin, daunorubicin, epirubicin, idarubicin, or valrubicin), and an optional pharmaceutical carrier and the treatment comprises administration of the monoclonal antibody, or antigen binding fragment thereof, in combination with a second and third composition, wherein the third composition comprises a hypomethylating agent, and an optional pharmaceutically acceptable carrier. In some aspects, the second composition comprises doxorubicin, and an optional pharmaceutical carrier. In some aspects, the hypomethylating agent is selected from azacitidine, and decitibine. In some aspects, the third composition comprises azacitidine, and an optional pharmaceutical carrier.

In some aspects, the second composition comprises a proteasome inhibitor, and an optional pharmaceutical carrier. In some aspects, the proteasome inhibitor is selected from bortezomib, carfilzomib, and ixazomib. In some aspects, the second composition comprises bortezomib, and an optional pharmaceutical carrier.

In some aspects, the second composition comprises a proteasome inhibitor (e.g., bortezomib, carfilzomib, or ixazomib), and an optional pharmaceutical carrier and the treatment comprises administration of the monoclonal antibody, or antigen binding fragment thereof, in combination with a second and third composition, wherein the third composition comprises a hypomethylating agent, and an optional pharmaceutically acceptable carrier. In some aspects, the second composition comprises bortezomib, and an optional pharmaceutical carrier. In some aspects, the hypomethylating agent is selected from azacitidine, and decitibine. In some aspects, the third composition comprises azacitidine, and an optional pharmaceutical carrier.

In some aspects, the second composition comprises a platinum derivative, and an optional pharmaceutical carrier. In some aspects, the platinum derivative is selected from oxaliplatin, carboplatin, and cisplatin. In some aspects, the second composition comprises oxaliplatin, and an optional pharmaceutical carrier.

In some aspects, the second composition comprises a platinum derivative (e.g., oxaliplatin, carboplatin, and cisplatin), and an optional pharmaceutical carrier, and the treatment comprises administration of the monoclonal antibody, or antigen binding fragment thereof, in combination with a second and third composition, wherein the third composition comprises a hypomethylating agent, and an optional pharmaceutically acceptable carrier. In some aspects, the second composition comprises oxaliplatin, and an optional pharmaceutical carrier. In some aspects, the hypomethylating agent is selected from azacitidine, and decitibine. In some aspects, the third composition comprises azacitidine, and an optional pharmaceutical carrier.

In some aspects, the second composition comprises an agent that inhibits a DNA damage response pathway, and an optional pharmaceutical carrier. In some aspects, the agent that inhibits a DNA damage response pathway is selected from an inhibitor of poly ADP ribose polymerase (PARP), wherein the inhibitor of PARP is Olaparib, Niraparib or Rucaparib. In some aspects, the second composition comprises Olaparib, and an optional pharmaceutical carrier.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47, or antigen binding fragment thereof, comprises:

a heavy chain complementarity determining region 1 (HC CDR1) comprising the amino acid sequence set forth in SEQ ID NO: 5;

a heavy chain complementarity determining region 2 (HC CDR2) comprising the amino acid sequence set forth in SEQ ID NO: 6;

a heavy chain complementarity determining region 3 (HC CDR3) comprising the amino acid sequence set forth in SEQ ID NO: 7;

a light chain complementarity determining region 1 (LC CDR1) comprising the amino acid sequence set forth in SEQ ID NO: 8;

a light chain complementarity determining region 2 (LC CDR2) comprising the amino acid sequence set forth in SEQ ID NO: 9; and

a light chain complementarity determining region 3 (LC CDR3) comprising the amino acid sequence set forth in SEQ ID NO: 10, for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises an inhibitor of BCL-2, and an optional pharmaceutically acceptable carrier. In some aspects, the inhibitor of BCL-2 is selected from Venetoclax™, Navitoclax, or obatoclax. In some aspects, the second composition comprises Venetoclax™, and an optional pharmaceutically acceptable carrier.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47, or antigen binding fragment thereof, comprises a heavy chain variable region (VH) comprising the amino acid sequence set forth in SEQ ID NO: 3 and a light chain variable region (VL) comprising the amino acid sequence set forth in SEQ ID NO: 4, for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises an inhibitor of BCL-2, and an optional pharmaceutically acceptable carrier. In some aspects, the inhibitor of BCL-2 is selected from Venetoclax™, Navitoclax, or obatoclax. In some aspects, the second composition comprises Venetoclax™, and an optional pharmaceutically acceptable carrier. In some aspects, the monoclonal antibody comprises a wild-type human IgG4 heavy chain constant region. In some aspects, the wild-type human IgG4 heavy chain constant region comprises the amino acid sequence set forth in SEQ ID NO: 21. In some aspects, the monoclonal antibody comprises a human kappa constant region. In some aspects, the human kappa constant region comprises the amino acid sequence set forth in SEQ ID NO: 17.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47 comprises a heavy chain comprising the amino acid sequence set forth in SEQ ID NO: 13; and a light chain comprising the amino acid sequence set forth in SEQ ID NO: 16.

for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises an inhibitor of BCL-2, and an optional pharmaceutically acceptable carrier. In some aspects, the inhibitor of BCL-2 is selected from Venetoclax™, Navitoclax, or obatoclax. In some aspects, the second composition comprises Venetoclax™, and an optional pharmaceutically acceptable carrier.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47, or antigen binding fragment thereof, comprises:

a heavy chain complementarity determining region 1 (HC CDR1) comprising the amino acid sequence set forth in SEQ ID NO: 5;

a heavy chain complementarity determining region 2 (HC CDR2) comprising the amino acid sequence set forth in SEQ ID NO: 6;

a heavy chain complementarity determining region 3 (HC CDR3) comprising the amino acid sequence set forth in SEQ ID NO: 7;

a light chain complementarity determining region 1 (LC CDR1) comprising the amino acid sequence set forth in SEQ ID NO: 8;

a light chain complementarity determining region 2 (LC CDR2) comprising the amino acid sequence set forth in SEQ ID NO: 9; and

a light chain complementarity determining region 3 (LC CDR3) comprising the amino acid sequence set forth in SEQ ID NO: 10,

for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises an anthracycline, and an optional pharmaceutically acceptable carrier. In some aspects, the anthracycline is selected from doxorubicin, daunorubicin, epirubicin, idarubicin, and valrubicin. In some aspects, the second composition comprises doxorubicin, and an optional pharmaceutically acceptable carrier.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47, or antigen binding fragment thereof, comprises a heavy chain variable region (VH) comprising the amino acid sequence set forth in SEQ ID NO: 3 and a light chain variable region (VL) comprising the amino acid sequence set forth in SEQ ID NO: 4,

for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises an anthracycline, and an optional pharmaceutically acceptable carrier. In some aspects, the anthracycline is selected from doxorubicin, daunorubicin, epirubicin, idarubicin, and valrubicin. In some aspects, the second composition comprises doxorubicin, and an optional pharmaceutically acceptable carrier. In some aspects, the monoclonal antibody comprises a wild-type human IgG4 heavy chain constant region. In some aspects, the wild-type human IgG4 heavy chain constant region comprises the amino acid sequence set forth in SEQ ID NO: 21. In some aspects, the monoclonal antibody comprises a human kappa constant region. In some aspects, the human kappa constant region comprises the amino acid sequence set forth in SEQ ID NO: 17.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47 comprises a heavy chain comprising the amino acid sequence set forth in SEQ ID NO: 13; and a light chain comprising the amino acid sequence set forth in SEQ ID NO: 16.

for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises an anthracycline, and an optional pharmaceutically acceptable carrier. In some aspects, the anthracycline is selected from doxorubicin, daunorubicin, epirubicin, idarubicin, and valrubicin. In some aspects, the second composition comprises doxorubicin, and an optional pharmaceutically acceptable carrier.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47, or antigen binding fragment thereof, comprises:

a heavy chain complementarity determining region 1 (HC CDR1) comprising the amino acid sequence set forth in SEQ ID NO: 5;

a heavy chain complementarity determining region 2 (HC CDR2) comprising the amino acid sequence set forth in SEQ ID NO: 6;

a heavy chain complementarity determining region 3 (HC CDR3) comprising the amino acid sequence set forth in SEQ ID NO: 7;

a light chain complementarity determining region 1 (LC CDR1) comprising the amino acid sequence set forth in SEQ ID NO: 8;

a light chain complementarity determining region 2 (LC CDR2) comprising the amino acid sequence set forth in SEQ ID NO: 9; and

a light chain complementarity determining region 3 (LC CDR3) comprising the amino acid sequence set forth in SEQ ID NO: 10,

for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises a proteasome inhibitor, and an optional pharmaceutically acceptable carrier. In some aspects, the proteasome inhibitor is selected from bortezomib, carfilzomib, and ixazomib. In some aspects, the second composition comprises bortezomib, and an optional pharmaceutically acceptable carrier.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47, or antigen binding fragment thereof, comprises a heavy chain variable region (VH) comprising the amino acid sequence set forth in SEQ ID NO: 3 and a light chain variable region (VL) comprising the amino acid sequence set forth in SEQ ID NO: 4, for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises a proteasome inhibitor, and an optional pharmaceutically acceptable carrier. In some aspects, the proteasome inhibitor is selected from bortezomib, carfilzomib, and ixazomib. In some aspects, the second composition comprises bortezomib, and an optional pharmaceutically acceptable carrier. In some aspects, the monoclonal antibody comprises a wild-type human IgG4 heavy chain constant region. In some aspects, the wild-type human IgG4 heavy chain constant region comprises the amino acid sequence set forth in SEQ ID NO: 21. In some aspects, the monoclonal antibody comprises a human kappa constant region. In some aspects, the human kappa constant region comprises the amino acid sequence set forth in SEQ ID NO: 17.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47 comprises a heavy chain comprising the amino acid sequence set forth in SEQ ID NO: 13; and a light chain comprising the amino acid sequence set forth in SEQ ID NO: 16, for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises a proteasome inhibitor, and an optional pharmaceutically acceptable carrier. In some aspects, the proteasome inhibitor is selected from bortezomib, carfilzomib, and ixazomib. In some aspects, the second composition comprises bortezomib, and an optional pharmaceutically acceptable carrier.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47, or antigen binding fragment thereof, comprises:

a heavy chain complementarity determining region 1 (HC CDR1) comprising the amino acid sequence set forth in SEQ ID NO: 5;

a heavy chain complementarity determining region 2 (HC CDR2) comprising the amino acid sequence set forth in SEQ ID NO: 6;

a heavy chain complementarity determining region 3 (HC CDR3) comprising the amino acid sequence set forth in SEQ ID NO: 7;

a light chain complementarity determining region 1 (LC CDR1) comprising the amino acid sequence set forth in SEQ ID NO: 8;

a light chain complementarity determining region 2 (LC CDR2) comprising the amino acid sequence set forth in SEQ ID NO: 9; and

a light chain complementarity determining region 3 (LC CDR3) comprising the amino acid sequence set forth in SEQ ID NO: 10,

for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises a platinum derivative, and an optional pharmaceutically acceptable carrier. In some aspects, the platinum derivative is selected from oxaliplatin, carboplatin, and cisplatin. In some aspects, the second composition comprises oxaliplatin, and an optional pharmaceutically acceptable carrier.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47, or antigen binding fragment thereof, comprises a heavy chain variable region (VH) comprising the amino acid sequence set forth in SEQ ID NO: 3 and a light chain variable region (VL) comprising the amino acid sequence set forth in SEQ ID NO: 4, for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises a platinum inhibitor, and an optional pharmaceutically acceptable carrier. In some aspects, the platinum derivative is selected from oxaliplatin, carboplatin, and cisplatin. In some aspects, the second composition comprises oxaliplatin, and an optional pharmaceutically acceptable carrier. In some aspects, the monoclonal antibody comprises a wild-type human IgG4 heavy chain constant region. In some aspects, the wild-type human IgG4 heavy chain constant region comprises the amino acid sequence set forth in SEQ ID NO: 21. In some aspects, the monoclonal antibody comprises a human kappa constant region. In some aspects, the human kappa constant region comprises the amino acid sequence set forth in SEQ ID NO: 17.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47 comprises a heavy chain comprising the amino acid sequence set forth in SEQ ID NO: 13; and a light chain comprising the amino acid sequence set forth in SEQ ID NO: 16,

for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises a platinum derivative, and an optional pharmaceutically acceptable carrier. In some aspects, the platinum derivative is selected from oxaliplatin, carboplatin, and cisplatin. In some aspects, the second composition comprises oxaliplatin, and an optional pharmaceutically acceptable carrier.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47, or antigen binding fragment thereof, comprises:

a heavy chain complementarity determining region 1 (HC CDR1) comprising the amino acid sequence set forth in SEQ ID NO: 5;

a heavy chain complementarity determining region 2 (HC CDR2) comprising the amino acid sequence set forth in SEQ ID NO: 6;

a heavy chain complementarity determining region 3 (HC CDR3) comprising the amino acid sequence set forth in SEQ ID NO: 7;

a light chain complementarity determining region 1 (LC CDR1) comprising the amino acid sequence set forth in SEQ ID NO: 8;

a light chain complementarity determining region 2 (LC CDR2) comprising the amino acid sequence set forth in SEQ ID NO: 9; and

a light chain complementarity determining region 3 (LC CDR3) comprising the amino acid sequence set forth in SEQ ID NO: 10,

for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises an inhibitor of PARP, and an optional pharmaceutically acceptable carrier. In some aspects, the inhibitor of PARP is selected from Olaparib, Niraparib and Rucaparib. In some aspects, the second composition comprises Olaparib, and an optional pharmaceutically acceptable carrier.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47, or antigen binding fragment thereof, comprises a heavy chain variable region (VH) comprising the amino acid sequence set forth in SEQ ID NO: 3 and a light chain variable region (VL) comprising the amino acid sequence set forth in SEQ ID NO: 4,

for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises an inhibitor of PARP, and an optional pharmaceutically acceptable carrier. In some aspects, the inhibitor of PARP is selected from Olaparib, Niraparib and Rucaparib. In some aspects, the second composition comprises Olaparib, and an optional pharmaceutically acceptable carrier. In some aspects, the monoclonal antibody comprises a wild-type human IgG4 heavy chain constant region. In some aspects, the wild-type human IgG4 heavy chain constant region comprises the amino acid sequence set forth in SEQ ID NO: 21. In some aspects, the monoclonal antibody comprises a human kappa constant region. In some aspects, the human kappa constant region comprises the amino acid sequence set forth in SEQ ID NO: 17.

In other aspects, the disclosure provides a composition comprising a monoclonal antibody that specifically binds human CD47, or an antigen binding fragment thereof, and an optional pharmaceutically acceptable carrier, wherein the monoclonal antibody that specifically binds human CD47 comprises a heavy chain comprising the amino acid sequence set forth in SEQ ID NO: 13; and a light chain comprising the amino acid sequence set forth in SEQ ID NO: 16,

for use in treating or delaying progression of cancer or reducing or inhibiting tumor growth in a subject in need thereof, wherein the treatment comprises administration of the monoclonal antibody, or antigen binding fragment thereof, in combination with a second composition, wherein the second composition comprises an inhibitor of PARP, and an optional pharmaceutically acceptable carrier. In some aspects, the inhibitor of PARP is selected from Olaparib, Niraparib and Rucaparib. In some aspects, the second composition comprises Olaparib, and an optional pharmaceutically acceptable carrier.

In any of the foregoing or related aspects, the subject has a cancer or tumor, wherein the cancer or tumor is a hematological cancer or hematological tumor. In some aspects, the hematological cancer or tumor is chronic lymphocytic leukemia (CLL), acute lymphocytic leukemia (ALL), chronic myelogenous leukemia (CML), diffuse large cell B cell lymphoma (DLBCL), follicular lymphoma (FL), Non-Hodgkin's lymphoma, myelofibrosis, mastocytosis, mantle cell lymphoma, multiple myeloma (MM) or acute myeloid leukemia (AML).

In other aspects, the disclosure provides use of a monoclonal antibody composition of the disclosure, and an optional pharmaceutically acceptable carrier, in the manufacture of a first medicament for treating or delaying progression of cancer or reducing or inhibiting tumor growth in a subject in need thereof, wherein the first medicament comprises the monoclonal antibody and an optional pharmaceutically acceptable carrier, and wherein the treatment comprises administration of the medicament in combination with a second composition, and, optionally a third composition, wherein the second composition comprises a cell-death inducing agent, and an optional pharmaceutically acceptable carrier, wherein the cell death-inducing agent is selected from an agent that induces apoptosis, an agent that induces immunogenic cell death (ICD), and an agent that inhibits a DNA damage response pathway, optionally in combination with a third composition, wherein the third composition comprises a hypomethylating agent and an optional pharmaceutically acceptable carrier.

Other aspects of the disclosure provide kits comprising a medicament comprising a monoclonal antibody composition of the disclosure, and an optional pharmaceutically acceptable carrier, and a package insert comprising instructions for administration of the medicament in combination with a second medicament comprising a composition comprising a cell-death inducing agent, and an optional pharmaceutically acceptable carrier, for treating or delaying progression of cancer or reducing or inhibiting tumor growth in a subject in need thereof, wherein the cell death-inducing agent is selected from an agent that induces apoptosis, an agent that induces immunogenic cell death (ICD), and an agent that inhibits a DNA damage response pathway, optionally comprising instructions for administration of the medicament in combination with a third medicament comprising a hypomethylating agent, and an optional pharmaceutically acceptable carrier.

In other aspects, the disclosure provides a kit comprising a container comprising a monoclonal antibody composition of the disclosure, and an optional pharmaceutically acceptable carrier, and a package insert comprising instructions for administration of the composition in combination with a second composition comprising a cell-death inducing agent, and an optional pharmaceutically acceptable carrier, for treating or delaying progression of cancer or reducing or inhibiting tumor growth in a subject in need thereof, wherein the cell death-inducing agent is selected from an agent that induces apoptosis, an agent that induces immunogenic cell death (ICD), and an agent that inhibits a DNA damage response pathway, optionally comprising instructions for administration of the medicament in combination with a third medicament comprising a hypomethylating agent, and an optional pharmaceutically acceptable carrier.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The patent or application file contains at least one drawing executed in color. Copies of this patent or patent application publication with color drawing(s) will be provided by the Office upon request and payment of the necessary fee.

FIG. 1 is a graph showing the percentage of dead non-phagocytosed Jurkat cells (CFSE+CD14-) as analyzed by flow cytometry from co-cultures with macrophages after treatment with anti-CD47 antibodies 2.3D11 or B6H12, and their respective isotype controls.

FIG. 2 is a graph showing the percentage of phagocytosed Jurkat cells (CSFE+CD14+) as analyzed by flow cytometry from co-cultures with macrophages after treatment with anti-CD47 antibodies 2.3D11 or B6H12, and their respective isotype controls.

FIGS. 3A and 3B show that antibody 2.3D11-induced cell death is caspase-independent, but dependent on PLCγ1. FIG. 3A graphically depicts that the effect of the pan-caspase inhibitor Z-VAD-FMK on antibody 2.3D11 induced cell death. FIG. 3B graphically depicts the effects of PLCγ1 expression on antibody 2.3D11 induced cell death.

FIGS. 4A-4E show the anti-tumor efficacy of the combination of anti-CD47 antibody 2.3D11 and BCL-2 inhibitor ABT-199. FIGS. 4A-4D provides graphs showing tumor volumes in individual mice with Ri-1 tumors treated with isotype control (top left), ABT-199 (top right), 2.3D11 (bottom left) or 2.3D11+ABT-199 (bottom right). FIG. 4E is a graph showing the mean (+95% confidence interval) tumor volumes of mice described in FIGS. 4A-4D

FIGS. 5A and 5B show the anti-tumor efficacy of the combination of anti-CD47 antibody 2.3D11 and BCL-2 inhibitor ABT-199 in a MOLM-13 (AML) xenograft model. FIG. 5A provides graphs showing tumor volumes in mice treated with isotype control, 10 μg 2.3D11, 50 mg/kg ABT-199, and 10 μg 2.3D11 in combination with 50 mpk ABT-199. FIG. 5B provides graphs showing tumor volumes in mice treated with isotype control, 60 μg 2.3D11, 100 mg/kg ABT-199, and 60 μg 2.3D11 in combination with 100 mpk ABT-199.

FIG. 6 graphically depicts the anti-tumor efficacy of the combination of anti-CD47 antibody 2.3D11 and BCL-2 inhibitor ABT-199 in a HL-60 (AML) xenograft model. Mice were treated with isotype control, 30 μg 2.3D11, 100 mg/kg ABT-199, and 30 μg 2.3D11 in combination with 100 mpk ABT-199.

FIG. 7 graphically depicts the anti-tumor efficacy of the combination of anti-CD47 antibody 2.3D11 and BCL-2 inhibitor ABT-199 in an OPM2 (Human Multiple Myeloma) xenograft model. Mice were treated with isotype control, 30 μg 2.3D11, 100 mg/kg ABT-199, and 30 μg 2.3D11 in combination with 100 mpk ABT-199.

FIG. 8 graphically depicts the ability of the in vitro combination of anti-CD47 antibody 2.3D11 and BCL-2 inhibitor ABT-199 to induce cell death in HL-60 cells.

FIGS. 9A and 9B show the ability of the combination of anti-CD47 antibody 2.3D11 and BCL-2 inhibitor ABT-199 to effect the levels of the chemokines, MCP-1 and MIP-1a, in an HL-60 xenograft model. FIG. 9A provides a graph showing MCP-1 concentration (ng/ml) after mice were treated for three hours or 24 hours with isotype control, 100 μg 2.3D11, 100 mg/kg ABT-199, and 100 μg 2.3D11 in combination with 100 mg/kg ABT-199. FIG. 9B provides a graph showing MIP-1a concentration (ng/ml) after mice were treated for three hours or 24 hours with isotype control, 100 μg 2.3D11, 100 mg/kg ABT-199, and 100 μg 2.3D11 in combination with 100 mg/kg ABT-199.

FIGS. 9C and 9D show the ability of the combination of anti-CD47 antibody 2.3D11 and BCL-2 inhibitor ABT-199 to effect the levels of the cytokines TNFα and IL-1β in an HL-60 xenograft model. FIG. 9C provides a graph showing TNFα concentration (ng/ml) after mice were treated for three hours or 24 hours with isotype control, 100 μg 2.3D11, 100 mg/kg ABT-199, and 100 μg 2.3D11 in combination with 100 mg/kg ABT-199. FIG. 9D provides a graph showing IL-1β concentration (ng/ml) after mice were treated for three hours or 24 hours with isotype control, 100 μg 2.3D11, 100 mg/kg ABT-199, and 100 μg 2.3D11 in combination with 100 mg/kg ABT-199.

FIG. 9E graphically depicts the ability of the combination of anti-CD47 antibody 2.3D11 and BCL-2 inhibitor ABT-199 to effect the levels of cleaved caspase-3 in an HL-60 xenograft model.

FIGS. 10A, 10B, and 10C show the ability of the combination of anti-CD47 antibody 2.3D11 and BCL-2 inhibitor ABT-199 to induces cell death in Jurkat cells (human malignant T cells). FIG. 10A graphically depicts necrotic cells showing positive staining for both Annexin V and PI. FIG. 10B graphically depicts apoptotic cells that are Annexin V positive and PI negative. FIG. 10C. graphically depicts apoptotic and necrotic cells by total AnnexinV+staining.

FIGS. 11A, 11B, and 11C show the ability of the combination of anti-CD47 antibody 2.3D11 and oxaliplatin to induces cell death in Jurkat cells (human malignant T cells). FIG. 11A graphically depicts necrotic cells showing positive staining for both Annexin V and PI. FIG. 11B graphically depicts apoptotic cells that are Annexin V positive and PI negative. FIG. 11C. graphically depicts apoptotic and necrotic cells by total AnnexinV+staining.

FIGS. 12A, 12B, and 12C show the ability of the combination of anti-CD47 antibody 2.3D11 and bortezomib to induces cell death in Jurkat cells (human malignant T cells). FIG. 12A graphically depicts necrotic cells showing positive staining for both Annexin V and PI. FIG. 12B graphically depicts apoptotic cells that are Annexin V positive and PI negative. FIG. 12C. graphically depicts apoptotic and necrotic cells by total AnnexinV+staining.

FIG. 13 graphically depicts the anti-tumor efficacy of the combination of anti-CD47 antibody 2.3D11 and proteasome inhibitor Velcade® (bortezomib) in an OPM2 (Human Multiple Myeloma) xenograft model. Mice were treated with isotype control, 30 μg 2.3D11, 0.5 mg/kg Velcade®, and 30 μg 2.3D11 in combination with 0.5 mg/kg Velcade®.

FIG. 14 graphically depicts the survival proportions of mice treated with the combination of anti-CD47 antibody 2.3D11 and the PARP inhibitor Olaparib. Mice were treated with isotype control, 100 μg 2.3D11, 50 mg/kg Olaparib, and 100 μg 2.3D11 in combination with 50 mg/kg Olaparib.

### DETAILED DESCRIPTION

### Overview

The present disclosure relates to methods and compositions for treating cancer in a subject. The disclosure is based, at least in part, upon the discovery that treatment of cancer by administering a monoclonal antibody that specifically binds to human CD47 is enhanced when the antibody is administered in combination with a cell death-inducing agent, e.g., an apoptosis-inducing agent, e.g., a Bcl-2 inhibitor. Without being bound by theory, it is believed that induction of cell death by one agent and induction of phagocytosis or other anti-tumor activity by the anti-CD47 agent bring together two, non-overlapping mechanisms to deepen anti-tumor effect and lower the probability of tumor escape by a resistance mechanism.

Specifically, it was discovered that combination of an anti-CD47 antibody (2.3D11) with a cell death-inducing agent, ABT-199 (also referred to herein as Venetoclax™), lead to synergistic enhancements in anti-tumor effects and decreased resistance to the cell death-inducing drug. Accordingly, in some aspects, the present disclosure provides a combination therapy in which apoptosis of tumor cells is induced by both an anti-CD47 monoclonal antibody (e.g., 2.3D11) and a cell death-inducing agent (e.g., ABT-199). Without being bound by theory it is believed that distinct events that lead to apoptosis via the intrinsic or extrinsic pathway could synergize to deepen the effect on tumor cell death. Several approved agents (including ABT-199) target the Bcl-2 family of anti-apoptotic molecules to trigger or lower the threshold for triggering intrinsic, caspase-dependent cell death. Anti-CD47 antibodies may target these same pathways or distinct pathways, such as the extrinsic and/or caspase-independent pathways to further lower the threshold for apoptosis (see Mateo et al. Nat Med 1999 5(11):1277-1284 and Bras et al. Mol Cell Biot 2007 27(20):7073-7088). It is believed that impinging on non-overlapping pathways to the same cellular outcome (tumor cell death) may increase molecular pressure on the tumor in a manner that lowers the probability of drug-resistance.

In other aspects, the present disclosure provides a combination therapy in which CD47 expression or availability on a tumor is upregulated by administration of a cell death-inducing agent (e.g., an apoptosis-inducing agent), such that cancer-specific targeting of the tumor by the anti-CD47 antibody is enhanced. Without being bound by theory, it is believed that a cell death-inducing agent may lead to preferential cell death of tumor cells and upregulation of CD47 thereby enhancing targeting of the anti-CD47 antibody to the tumor.

In other aspects, the present disclosure provides a combination therapy in which administration of a cell death-inducing agent may lead to release of soluble CD47 or membrane-bound fragments or exosomes containing CD47 that could act as an immunosuppressive cloud to engage SIRPα and limit myeloid cell responsiveness. Without being bound by theory, it is believed that providing CD47 blockade (e.g., an anti-CD47 antibody) in these contexts may relieve the immunosuppression and thereby deepen the anti-tumor effect of the cell death-inducing agent.

In other aspects, the present disclosure provides a combination therapy in which early events associated with subsequent cell death, stimulated by the cell death-inducing agent, may facilitate phagocytosis, which is further enhanced by the other pro-phagocytic functions of administration of an anti-CD47 antibody. Without being bound by theory, it is believed that during these early events, molecules on the surface of tumor cells may be increased or exposed that act as ‘eat me’ signals to enhance phagocytosis. The concomitant upregulation of ‘eat me’ signals (e.g., calreticulin), by the cell death-inducing agent, with the blockade of the ‘don't eat me’ signal provided by an anti-CD47 antibody may synergize to enhance phagocytosis and the subsequent presentation of tumor antigen to T cells to drive not only tumor cell removal, but also anti-tumor immunity.

Accordingly, in one aspect, the disclosure provides a method of treating or delaying progression of cancer in a subject in need thereof by administering an effective amount of a monoclonal antibody that specifically binds human CD47 and an effective amount of a cell death-inducing agent, e.g., an apoptosis inducing agent, e.g., a Bcl-2 inhibitor. In some embodiments, the cancer comprises cells that express CD47.

The present disclosure is also based, at least in part, on the discovery that an anti-CD47 antibody which induces tumor cell death, when combined with another cell death inducing agent, cooperate to induce cell death and promote an enhanced anti-tumor inflammatory response. As described herein, it has been shown that antibody 2.3D11 (having the heavy and light chain amino acid sequences set forth in SEQ ID Nos 13 and 16), which was previously shown to potently inhibit the interaction between CD47 and SIRPα and enhance phagocytosis of tumor cells, induces cell death of CD47 expressing cells via a pathway which is caspase independent and partially dependent on PLCγ1 expression. It is believed that antibody 2.3D11 (having the heavy and light chain amino acid sequences set forth in SEQ ID Nos 13 and 16) induces cell death in a manner that enhances inflammatory responses via tumor intrinsic cell death mechanisms and tumor cell extrinsic mechanisms, including phagocytosis induction and production of inflammatory cytokines which result in recruitment of myeloid cells.

Accordingly, without being bound by theory, it is believed that a combination of an anti-CD47 antibody, such as antibody 2.3D11 (having the heavy and light chain amino acid sequences set forth in SEQ ID Nos 13 and 16), with another cell death inducing agent, such as an apoptosis inducing agent (e.g., a BCL-2 inhibitor) or an immunogenic cell death (ICD) inducing agent (e.g., an anthracycline, a proteasome inhibitor, or a platinum derivative) results in enhanced cell death via non-overlapping mechanisms (e.g., caspase-independent and caspase dependent) or overlapping (e.g., ICD). As described herein, it has been shown that a combination of antibody 2.3D11 (having the heavy and light chain amino acid sequences set forth in SEQ ID Nos 13 and 16) with a BCL-2 inhibitor, ABT-199, which induces cell death in a caspase-dependent manner, synergizes and significantly increases tumor levels of the monocyte chemoattractant protein 1 (MCP-1) and macrophage inflammatory protein 1-alpha (MIP-1α). Both chemokines are known to elicit an inflammatory response that leads to macrophage infiltration into the tumor. The combination was also shown to upregulate production of TNFα and IL-1β, suggesting the presence of immune cell infiltration. Unexpectedly, and in contrast to monotherapy, the level of cleaved caspase-3 in the tumor was significantly lower following administration of an antibody 2.3D11 (having the heavy and light chain amino acid sequences set forth in SEQ ID Nos 13 and 16) and ABT-199.

Without being bound by theory, these results suggest an acceleration of tumor cell clearance, likely due to the presence of recruited macrophages in the tumor. The 2.3D11-driven properties of inducing an alternative pathway of tumor intrinsic cell death induction, recruiting myeloid cells with associated production of inflammatory cytokines and increasing tumor clearance are all mechanisms whereby 2.3D11 (having the heavy and light chain amino acid sequences set forth in SEQ ID Nos 13 and 16) may cooperate with, augment, or enhance the properties of ABT-199 to deepen the anti-tumor response (e.g., augment, enhance, or increase the anti-tumor response). Significantly, the combination of antibody 2.3D11 (having the heavy and light chain amino acid sequences set forth in SEQ ID Nos 13 and 16) and ABT-199 was shown to have a synergistic effect in vivo by inhibiting tumor re-growth in ABT-199-sensitive models. Likewise, in a tumor cell line which expresses high levels of MCL-1 with reduced sensitivity to ABT-199, the combination of antibody 2.3D11 (having the heavy and light chain amino acid sequences set forth in SEQ ID Nos 13 and 16) and ABT-199 resulted in an enhanced anti-tumor activity. These results suggest that, while combination of antibody 2.3D11 (having the heavy and light chain amino acid sequences set forth in SEQ ID Nos 13 and 16) with ABT-199 in a cell line with reduced ABT-199 sensitivity may provide an anti-tumor benefit to patients, a combination of antibody 2.3D11 with inhibitors of other Bcl2 family members, such as MCL-1 inhibitor, may lead to improved outcomes.

It has also been shown that a combination of an anti-CD47 antibody and agents that induce immunogenic cell death by, for example, inducing calreticulin surface localization, maturation and enhanced functionality of antigen-presenting cells, result in enhanced cell death in vitro and enhanced anti-tumor efficacy in vivo. Specifically, combination of antibody 2.3D11 (having the heavy and light chain amino acid sequences set forth in SEQ ID Nos 13 and 16) and an anthracycline (doxorubicin), or a proteasome inhibitor (bortezomib) or a platinum derivative (oxaliplatin) was shown to enhance cell death. And, a combination of antibody 2.3D11 and bortezomib enhanced anti-tumor efficacy in a human multiple myeloma model. Borezomib or Velcade® is also known to induce cell death by upregulation of NOXA, a pro-apoptotic BH3 protein and trigger cleavage of MCL-1, caspase-3, caspase-9, and PARP. These results suggest that a combination of an anti-CD47 antibody with an immunogenic cell death inducing agent may provide an enhanced anti-tumor benefit to patients relative to either compound individually.

It has also been shown that a combination of an anti-CD47 antibody and an agent which induces cell death by inhibiting the DNA damage response pathway and/or DNA repair pathway in tumor cells enhanced anti-tumor efficacy in vivo. Specifically, a combination of antibody 2.3D11 (having the heavy and light chain amino acid sequences set forth in SEQ ID Nos 13 and 16) and a PARP inhibitor, Olaparib, was shown to prolong survival in a human intraperitoneal ovarian model. These results suggest that a combination of an anti-CD47 antibody with PARP inhibitor may provide an enhanced anti-tumor benefit to patients relative to either compound individually. Without being bound by theory, it is believed that Olaparib enhancing DNA damage/tumor cell stress results in increased sensitivity of tumor cells to anti-CD47 antibody induced cell death. PARP inhibition maintains the expression of inflammatory mediators such as IL-1, IL-6, IL-8, CCL3, TNF-a and IFN-gamma and reduces the expression of adhesion molecules, including ITAM-1 and VCAM which can affect the homeostasis of immune cells. PARP-1 inhibition may also diminish the immune response. Therefore, the combination of antibody 2.3D11 (having the heavy and light chain amino acid sequences set forth in SEQ ID Nos 13 and 16) may restore, promote, or enhance anti-tumor immune responses. In addition, DNA damage and cell death triggered by PARP-1 inhibitors may enhance the phagocytic activity of anti-CD47 antibodies, such as 2.3D11 (having the heavy and light chain amino acid sequences set forth in SEQ ID Nos 13 and 16) resulting in enhanced anti-tumor immune responses.

Accordingly, the present disclosure provides a methods and compositions for treating or delaying progression of cancer or reducing or inhibiting tumor growth in a subject in need thereof, by administering to the subject an effective amount of a monoclonal antibody that specifically binds human CD47, or antigen binding fragment thereof, and an effective amount of a cell death-inducing agent, wherein the cell death-inducing agent is selected from an agent that induces apoptosis, an agent that induces immunogenic cell death (ICD), and an agent that inhibits a DNA damage response pathway. These and other embodiments of the disclosure are described in detail herein.

### Definitions

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g.: Sambrook J. & Russell D. Molecular Cloning: A Laboratory Manual, 3rd ed., Cold Spring Harbor Laboratory Press, Cold Spring Harbor, N.Y. (2000); Ausubel et al., Short Protocols in Molecular Biology: A Compendium of Methods from Current Protocols in Molecular Biology, Wiley, John & Sons, Inc. (2002); Harlow and Lane Using Antibodies: A Laboratory Manual; Cold Spring Harbor Laboratory Press, Cold Spring Harbor, N.Y. (1998); and Coligan et al., Short Protocols in Protein Science, Wiley, John & Sons, Inc. (2003). Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

The term “ameliorating” refers to any therapeutically beneficial result in the treatment of a disease state, e.g., cancer, including prophylaxis, lessening in the severity or progression, remission, or cure thereof.

As used herein, an “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, larger “peptide insertions,” can also be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

As used herein, the term “antagonist” refers to any molecule that partially or fully blocks, inhibits, suppresses, abrogates, interferes, and/or neutralizes a biological activity of a native polypeptide disclosed herein. Suitable antagonist molecules specifically include antagonist antibodies or antibody fragments, fragments or amino acid sequence variants of native polypeptides, peptides, nucleic acids (e.g., antisense oligonucleotides), small organic molecules, carbohydrates, carbohydrate derivatives, lipids (e.g., phospholipids) etc. In some embodiments, inhibition in the presence of the antagonist is observed in a dose-dependent manner. In some embodiments, the measured signal (e.g., biological activity) is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100% lower than the signal measured with a negative control under comparable conditions. Also disclosed herein, are methods of identifying antagonists suitable for use in the methods of the disclosure. For example, these methods include, but are not limited to, binding assays such as enzyme-linked immuno-absorbent assay (ELISA), Forte Bio© systems, and radioimmunoassay (RIA). These assays determine the ability of an antagonist to bind the target polypeptide of interest (e.g., a receptor or ligand) and therefore indicate the ability of the antagonist to inhibit, neutralize or block the activity of the polypeptide. Efficacy of an antagonist can also be determined using functional assays, such as the ability of an antagonist to inhibit the function of the polypeptide or an agonist. For example, a functional assay may comprise contacting a polypeptide with a candidate antagonist molecule and measuring a detectable change in one or more biological activities normally associated with the polypeptide. The potency of an antagonist is usually defined by its IC₅₀ value (concentration required to inhibit 50% of the agonist response). The lower the IC₅₀ value the greater the potency of the antagonist and the lower the concentration that is required to inhibit the maximum biological response.

The polypeptides suitable for use in the methods disclosed herein may comprise conservative amino acid substitutions at one or more amino acid residues, e.g., at essential or non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In certain embodiments, a string of amino acids can be replaced with a structurally similar string that differs in order and/or composition of side chain family members. Alternatively, in certain embodiments, mutations may be introduced randomly along all or part of a coding sequence, such as by saturation mutagenesis, and the resultant mutants can be incorporated into binding polypeptides and/or molecules of the invention and screened for their ability to bind to the desired target.

Throughout this specification and claims, the word “comprise,” or variations such as “comprises” or “comprising,” will be understood to imply the inclusion of a stated integer or group of integers but not the exclusion of any other integer or group of integers. Further, as used herein, the term “includes” means includes without limitation.

As used herein, the term “antibody” (Ab), which is synonymous with the term “immunoglobulin” (Ig), means a tetramer comprising two heavy (H) chains (about 50-70 kDa) and two light (L) chains (about 25 kDa) inter-connected by disulfide bonds. There are two types of light chain: λ, and K. In humans they are similar, but only one type is present in each antibody. Heavy chains are classified as mu, delta, gamma, alpha, or epsilon, and define the antibody's isotype as IgM, IgD, IgG, IgA, and IgE1 respectively. See generally, Fundamental Immunology Ch. 7 (Paul, W., ed., 2nd ed. Raven Press, N.Y. (1989)). Each heavy chain (herein sometimes referred to as H-chain or Hc) is comprised of a heavy chain variable domain (VH, or H-variable domain) and a heavy chain constant region (CH). The heavy chain constant region is comprised of three domains, CH1, CH2 and CH3. Each light chain (herein sometimes referred to as L-chain or Lc) is comprised of a light chain variable domain (VL, or L-variable domain) and a light chain constant region. The light chain constant region is comprised of one domain, CL. Within light and heavy chains, the variable and constant regions are joined by a “J” region of about 12 or more amino acids, with the heavy chain also including a “D” region of about 3 or more amino acids. The VH and VL regions can be further subdivided into regions of hypervariability, termed “complementarity determining regions” (CDR), interspersed with regions that are more conserved, termed “framework regions” (FR). Each VH and VL is composed of three CDRs (H-CDR herein designates a CDR from the heavy chain; and L-CDR herein designates a CDR from the light chain) and four FRs, arranged from amino-terminus to carboxyl-terminus in the following order: FR1, CDR1, FR2, CDR2, FR3, CDR3, FR4. The assignment of amino acids to each domain is in accordance with the definitions of Kabat, Sequences of Proteins of Immunological Interest (National Institutes of Health, Bethesda, Md. (1987 and 1991)), or Chothia & Lesk, J. MoI. Biol. 196:901-917 (1987); Chothia et al., Nature 342:878-883 (1989).

As used herein, the term “affinity” means a measure of the attraction between an antigen and an antibody.

As used herein, the term “antigen-binding fragment” refers to one or more fragments of an antibody that retain the ability to specifically bind to an antigen. It has been shown that the antigen-binding function of an antibody can be performed by fragments of a full-length antibody. Non-limiting examples of binding fragments encompassed within the term “antigen-binding fragment” include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment which is obtained by cleaving a disulfide bond of the hinge region of the F(ab′)2; (iv) a Fd fragment consisting of the VH and CH1 domains; (v) a Fv fragment consisting of the VL and VH domains of a single arm of an antibody, (vi) a dAb fragment (Ward et al., (1989) Nature 341:544-546), which consists of a VH domain; (vii) an isolated complementarity determining region (CDR); and (viii) a dsFv, which consists of a VH::VL heterodimer stabilized by a disulfide bond. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al. Science 242:423-426 (1988) and Huston et al. Proc. Natl. Acad. Sci. USA 85:5879-5883 (1988)). Also within the scope of this disclosure are antigen-binding molecules comprising a VN and/or a VL, In the case of a VH, the molecule may also comprise one or more of a CH 1, hinge, CH2 or CH3 region. Such single chain antibodies are also intended to be encompassed within the term “antigen-binding fragment” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. Proc. Natl. Acad. Sci. USA 90:6444-6448 (1993); Poljak et al. Structure 2:1121-1123 (1994)).

As used herein, the term “antibody fragment” also includes, e.g., single domain antibodies such as camelized single domain antibodies. See, e.g., Muyldermans et al. (2001) Trends Biochem Sci 26:230-235; Nuttall et al. (2000) Curr Pharm Biotech 1:253-263; Reichmann et al. (1999) J Immunol Meth 231:25-38; PCT application publication nos. WO94/04678 and WO 94/25591; and U.S. Pat. No. 6,005,079, all of which are incorporated herein by reference in their entireties. In some embodiments, the disclosure provides single domain antibodies comprising two VH domains with modifications such that single domain antibodies are formed.

As used herein, the term “apoptosis-inducing agent” refers to chemical or biological agent that, upon contact, induces apoptosis resulting in cell death by a programmed sequence of events. Hallmarks of apoptosis include, but are not limited to, morphological changes, cell shrinkage, nuclear and cytoplasmic condensation, and alterations in plasma membrane topology. Biochemically, apoptotic cells are characterized by increased intracellular calcium concentration, fragmentation of chromosomal DNA, and expression of novel cell surface components.

As used herein, the term “bispecific” or “bifunctional antibody” refers to an artificial hybrid antibody having two different heavy/light chain pairs and two different binding sites. Bispecific antibodies can be produced by a variety of methods including fusion of hybridomas or linking of Fab′ fragments. See, e.g., Songsivilai & Lachmann, (1990) Clin. Exp. Immunol. 79:315-321; Kostelny et al., (1992) J. Immunol. 148:1547-1553.

Traditionally, the recombinant production of bispecific antibodies is based on the co-expression of two immunoglobulin heavy-chain/light-chain pairs, where the two heavy chain/light-chain pairs have different specificities (Milstein and Cuello, (1983) Nature 305:537-539). Antibody variable domains with the desired binding specificities (antibody-antigen combining sites) can be fused to immunoglobulin constant domain sequences. The fusion of the heavy chain variable region is preferably with an immunoglobulin heavy-chain constant domain, including at least part of the hinge, CH2, and CH3 regions. For further details of illustrative currently known methods for generating bispecific antibodies see, e.g., Suresh et al., (1986) Methods Enzymol. 121:210; PCT Publication No. WO 96/27011; Brennan et al., (1985) Science 229:81; Shalaby et al., J. Exp. Med. (1992) 175:217-225; Kostelny et al., (1992) 1 Immunol. 148(5):1547-1553; Hollinger et al., (1993) Proc. Natl. Acad. Sci. USA 90:6444-6448; Gruber et al., (1994) J. Immunol. 152:5368; and Tutt et al., (1991) J. Immunol. 147:60. Bispecific antibodies also include cross-linked or heteroconjugate antibodies. Heteroconjugate antibodies may be made using any convenient cross-linking methods. Suitable cross-linking agents are well known in the art, and are disclosed in U.S. Pat. No. 4,676,980, along with a number of cross-linking techniques.

Various techniques for making and isolating bispecific antibody fragments directly from recombinant cell culture have also been described. For example, bispecific antibodies have been produced using leucine zippers. See, e.g., Kostelny et al. (1992) J Immunol 148(5):1547-1553. The leucine zipper peptides from the Fos and Jun proteins may be linked to the Fab′ portions of two different antibodies by gene fusion. The antibody homodimers may be reduced at the hinge region to form monomers and then re-oxidized to form the antibody heterodimers. This method can also be utilized for the production of antibody homodimers. The “diabody” technology described by Hollinger et al. (1993) Proc Natl Acad Sci USA 90:6444-6448 has provided an alternative mechanism for making bispecific antibody fragments. The fragments comprise a heavy-chain variable domain (VH) connected to a light-chain variable domain (VL) by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (scFv) dimers has also been reported. See, e.g., Gruber et al. (1994) J Immunol 152:5368. Alternatively, the antibodies can be “linear antibodies” as described in, e.g., Zapata et al. (1995) Protein Eng. 8(10):1057-1062. Briefly, these antibodies comprise a pair of tandem Fd segments (VH-CH1-VH-CH1) which form a pair of antigen binding regions. Linear antibodies can be bispecific or monospecific.

Antibodies with more than two valencies (e.g., trispecific antibodies) are contemplated and described in, e.g., Tutt et al. (1991) J Immunol 147:60.

The disclosure also embraces variant forms of multi-specific antibodies such as the dual variable domain immunoglobulin (DVD-Ig) molecules described in Wu et al. (2007) Nat Biotechnol 25(11): 1290-1297. The DVD-Ig molecules are designed such that two different light chain variable domains (VL) from two different parent antibodies are linked in tandem directly or via a short linker by recombinant DNA techniques, followed by the light chain constant domain. Similarly, the heavy chain comprises two different heavy chain variable domains (VH) linked in tandem, followed by the constant domain CH1 and Fc region. Methods for making DVD-Ig molecules from two parent antibodies are further described in, e.g., PCT Publication Nos. WO 08/024188 and WO 07/024715. In some embodiments, the bispecific antibody is a Fabs-in-Tandem immunoglobulin, in which the light chain variable region with a second specificity is fused to the heavy chain variable region of a whole antibody. Such antibodies are described in, e.g., International Patent Application Publication No. WO 2015/103072.

In some embodiments, an antibody described herein can be formatted as a bispecific antibody.

As used herein, the term “cancer” means cells having the capacity for autonomous growth, i.e., an abnormal state or condition characterized by rapidly proliferating cell growth.

As used herein, the term “CDR” means a complementarity-determining region.

As used herein, the term “cell death-inducing agent” refers to a chemical, biological, or pharmacological agent, including those described herein (e.g., an agent that induces apoptosis, an agent that induces immunogenic cell death (ICD), an agent that inhibits a DNA damage response or a chemotherapeutic agent) that, upon contact, negatively impacts the viability of a cell, by inducing one or more cell death modalities, and results in a dying or dead cell. Functional classifications of cell death modalities (also referred to as “cell death subroutines” or “cell death pathways”) recognized in the art include, but are not limited to, anoikis, autophagic cell death, apoptosis, caspase-dependent intrinsic apoptosis, caspase-independent intrinsic apoptosis, cornification, entosis, excitotoxicity, extrinsic apoptosis by death receptors, extrinsic apoptosis by dependence receptors, immunogenic cell death (alternatively known as “immunogenic apoptosis”), mitotic catastrophe, necrosis, necroptosis, netosis, paraptosis, parthanatos, pyroptosis, and pyronecrosis. Further descriptions of cell death modalities, including their morphological and molecular definitions, are found in Kroemer et al., (2005) Cell Death Differ 12(Suppl 2):1463-1467; Kroemer et al., (2009) Cell Death Differ 16(1): 3-11; and Galluzzi et al., (2012) Cell Death Differ 19: 107-120, all of which are incorporated herein by reference in their entirety.

The Nomenclature Committee on Cell Death (NCCD) has proposed that a cell should be considered dead when any one of the following molecular or morphological criteria is met: (1) the cell has lost the integrity of its plasma membrane, as defined by the incorporation of vital dyes (e.g., PI) in vitro; (2) the cell, including its nucleus, has undergone complete fragmentation into discrete bodies (which are frequently referred to as ‘apoptotic bodies’); and/or (3) its corpse (or its fragments) has been engulfed by an adjacent cell in vivo. Thus, bona fide ‘dead cells’ would be different from ‘dying cells’ that have not yet concluded their demise (which can occur through a variety of biochemically distinct pathways) (Kroemer et al., (2009) Cell Death Differ 16(1): 3-11).

As used herein, the term “chemotherapeutic agent” (alternatively “cytotoxic chemotherapeutic agent”) refers to a chemical or pharmacological agent that is known to be of use in the treatment of cancer. Furthermore, as used herein, the term connotes those pharmacological agents that are generally cytotoxic, non-specific intracellular poisons, especially those that function to inhibit the process of cell division known as mitosis, and excludes pharmacological agents that more selectively target cellular components known to cause or contribute to the formation, development and/or maintenance of cancer. Chemotherapeutic agents can induce one or more cell death modalities including immunogenic cell death.

As used herein, the term “chimeric antibody” means a genetically engineered fusion of parts of an animal antibody, typically a mouse antibody, with parts of a human antibody. Chimeric antibodies are developed to reduce the human anti-animal antibody response elicited by animal antibodies, as they combine the specificity of the animal antibody with the efficient human immune system interaction of a human antibody.

As used herein, the term “chimeric antibody” means a genetically engineered fusion of parts of an animal antibody, typically a mouse antibody, with parts of a human antibody. Chimeric antibodies are developed to reduce a human anti-animal antibody response.

As used herein, the term “cross-reacts” refers to the ability of an antibody of the disclosure to bind to an antigen from a different species. For example, an antibody of the present disclosure which binds human CD47 may also bind another species of CD47. As used herein, cross-reactivity is measured by detecting a specific reactivity with purified antigen in binding assays (e.g., SPR, ELISA) or binding to, or otherwise functionally interacting with, cells physiologically expressing CD277. Methods for determining crossreactivity include standard binding assays as described herein, for example, by Biacore™ surface plasmon resonance (SPR) analysis using a Biacore™ 2000 SPR instrument (Biacore AB, Uppsala, Sweden), or flow cytometric techniques.

As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment or delay progression of cancer, as compared to the response obtained without administration of the agent. In some embodiments, a therapeutically effective amount is an amount of an agent to be delivered (e.g., a monoclonal antibody in combination with a cell-death inducing agent) that is sufficient, when administered to a subject with cancer, to treat, improve symptoms of, prevent, and/or delay progression of disease and/or condition.

As used herein, the term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from the extracellular domain of the target of interest (e.g., CD47) are tested for reactivity with the given antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

Also encompassed by the present disclosure are antibodies that bind the same epitope and/or antibodies that compete for binding to a target of interest (e.g., human CD47) with the antibodies described herein. Antibodies that recognize the same epitope or compete for binding can be identified using routine techniques. Such techniques include, for example, an immunoassay, which shows the ability of one antibody to block the binding of another antibody to a target antigen, i.e., a competitive binding assay. Competitive binding is determined in an assay in which the immunoglobulin under test inhibits specific binding of a reference antibody to a common target antigen, such as CD47. Numerous types of competitive binding assays are known, for example: solid phase direct or indirect radioimmunoassay (MA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least about 50-55%, 55-60%, 60-65%, 65-70% 70-75% or more.

Other techniques include, for example, epitope mapping methods, such as, xray analyses of crystals of antigen:antibody complexes which provides atomic resolution of the epitope and mass spectrometry combined with hydrogen/deuterium (H/D) exchange which studies the conformation and dynamics of antigen:antibody interactions. Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. The peptides are then regarded as leads for the definition of the epitope corresponding to the antibody used to screen the peptide library. For epitope mapping, computational algorithms have also been developed which have been shown to map conformational discontinuous epitopes.

As used herein, the term “FR” means a framework region.

As used herein, the term “HCDR” means a heavy chain complementarity-determining region.

As used herein, the term “humanized antibody” means an antibody that has variable region framework and constant regions from a human antibody but retains the CDRs of the animal antibody.

As used herein, the term “immunogenic cell death (“ICD”)” (alternatively known as “immunogenic apoptosis”) refers to a cell death modality wherein contact of a tumor cell with a chemical, biological, or pharmacological agent is associated with activation of one or more signaling pathways that induces the pre-mortem expression and emission of damaged-associated molecular pattern (DAMPs) molecules from the tumor cell, resulting in the increase of immunogenicity of the tumor cell and the death of the cell in an immunogenic manner (e.g., by phagocytosis). ICD is a form of cell death which induces endoplasmic reticulum (ER) stress and involves changes in the composition of the cell surface as well as the release of DAMPs that elevate the immunogenic potential of dying cells. DAMPs include calreticulin, heat-shock proteins, secreted amphoterin (HMGB1) and ATP. Following ICD induction, calreticulin is translocated to the surface of dying cell where it functions as an “eat me” signal for professional phagocytes. HSP70 and HSP90 are also translocated to the plasma membrane where they interact with antigen-presenting cell (APCs) and facilitate cross-presentation of tumor antigens with MHC class I molecules, resulting in a CD8+ T cell response. HMGB1 is released into the extracellular space where is binds Toll-like receptors on APCs and facilitates presentation of tumor antigens by dendritic cells (professional APCs) to T cells. ATP secretion recruits monocytes to the site of cell death. Changes associated with ICD of tumor or cancer cells can induce an effective anti-tumor immune response through activation, maturation and enhanced antigen presentation of dendritic cells and activation of a specific T cell response in a subject.

As used herein, the term “immunogenic cell death (“ICD”) inducing agent” refers to an agent that induces ICD or a chemical, biological, or pharmacological agent that induces an immunogenic cell death process, pathway, or modality in a tumor cell. In some embodiments, an immunogenic cell death inducing agent is, for example, an anthracycline (e.g., doxorubicin, daunorubicin, epirubicin, idarubicin, valrubicin), a platinum derivative (e.g., oxaliplatin, carboplatin, cisplatin) or a proteasome inhibitor (e.g., Bortezomib, carfilzomib, ixazomib).

As used herein, the term “in combination,” as used in connection with a therapeutic treatment, is understood to mean that two (or more) different treatments, for example, two (or more) therapeutic agents, are delivered to the subject during the course of the subject's affliction with the disorder, such that the effects of the treatments on the patient overlap at a point in time. In certain embodiments, the delivery of one treatment ends before the delivery of the other treatment begins. In certain other embodiments, the delivery of one treatment is still occurring when the delivery of the second begins, so that there is overlap in terms of administration. This is sometimes referred to herein as “simultaneous” or “concurrent delivery.” In certain embodiments, the reduction of a symptom, or other parameter related to the disorder upon delivery of a combination therapy is greater than what would be observed with one treatment delivered in the absence of the other.

As used herein, the terms “inhibits”, “blocks”, or “reduces” are used interchangeably and encompass both partial and complete inhibition/blocking as well as direct and allosteric inhibition/blocking. For example, the inhibition/blocking of CD47 reduces or alters the normal level or type of activity that occurs from CD47 in a given system in the absence of inhibition or blocking. As used herein, “inhibition”, “blocking”, or “reduces” are also intended to include any measurable decrease in biological function and/or activity of a target (e.g. CD47). For example, when an antibody, or an antigen-binding fragment thereof is in contact with the target as compared to the target not in contact with an antibody, an antigen-binding fragment. In some embodiments, an antibody, or antigen-binding fragment thereof, that targets CD47 inhibits or reduces CD47 function and/or activity in a given system by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%.

As used herein, the term “inhibits growth” (e.g., referring to cells) is intended to include any measurable decrease in the growth of a cell, e.g., the inhibition of growth of a cell by at least about 10%, 20%, 30%, 40%, 50%, 60%, 70%, 80%, 90%, 99%, or 100%.

As used herein, the term “inhibits re-growth” (e.g., inhibits tumor re-growth) is intended to include any measurable decrease in the re-growth of a tumor, e.g., the inhibition of re-growth of a tumor by at least about 10%, 20%, 30%, 40%, 50%, 60%, 70%, 80%, 90%, 99%, or 100%.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a combination of an antibody that specifically binds human CD47 and a cell death-inducing agent).

The term “in vivo” refers to processes that occur in a living organism.

As used herein, the term “ka” is intended to refer to the on rate constant for the association of an antibody with the antigen.

As used herein, the term “kd” is intended to refer to the off rate constant for the dissociation of an antibody from the antibody/antigen complex.

As used herein, the term “LCDR” means a light chain complementarity-determining region.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

As used herein, “parenteral administration,” “administered parenterally,” and other grammatically equivalent phrases, refer to modes of administration other than enteral and topical administration, usually by injection, and include, without limitation, intravenous, intranasal, intraocular, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion.

As used herein, the term “patient” includes human and other mammalian subjects that receive either prophylactic or therapeutic treatment.

The term “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the “percent identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present disclosure can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

The nucleic acid compositions of the present disclosure, while often in a native sequence (except for modified restriction sites and the like), from either cDNA, genomic or mixtures thereof may be mutated, in accordance with standard techniques to provide gene sequences. For coding sequences, these mutations, may affect amino acid sequence as desired. In particular, DNA sequences substantially homologous to or derived from native V, D, J, constant, switches and other such sequences described herein are contemplated (where “derived” indicates that a sequence is identical or modified from another sequence).

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see, e.g., Berge et al. (1977) J Pharm Sci 66:1-19).

As used herein, the terms “polypeptide”, “peptide”, and “protein” are used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “preventing” when used in relation to a condition, refers to administration of a composition which reduces the frequency of, or delays the onset of, symptoms of a medical condition in a subject relative to a subject which does not receive the composition.

As used herein, the term “purified” or “isolated” as applied to any of the proteins (antibodies or fragments) described herein refers to a polypeptide that has been separated or purified from components (e.g., proteins or other naturally-occurring biological or organic molecules) which naturally accompany it, e.g., other proteins, lipids, and nucleic acid in a prokaryote expressing the proteins. Typically, a polypeptide is purified when it constitutes at least 60 (e.g., at least 65, 70, 75, 80, 85, 90, 92, 95, 97, or 99) %, by weight, of the total protein in a sample.

As used herein, the term “recombinant host cell” (or simply “host cell”) is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the terms “specific binding,” “selective binding,” “selectively binds,” and “specifically binds,” refer to an antibody binding to an epitope on a predetermined antigen. Typically, the antibody or antagonist binds with an equilibrium dissociation constant (Kd) of approximately less than 10⁻⁶ M, such as approximately less than 10⁻⁷, 10⁻⁸M, 10⁻⁹ M or 10⁻¹⁰ M or even lower when determined by surface plasmon resonance (SPR) technology in a BIACORE 2000 instrument. For example, when using the recombinant human CD47 extracellular domain as the analyte/predetermined antigen and an antibody that specifically binds human CD47 as the ligand, SPR can be used to measure binding of the ligand/antibody to the analyte/predetermined antigen with an affinity that is at least about two-fold greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.”

As used herein, the term “sequential dosage” and related terminology refers to the administration of at least one agent (e.g., an antibody that specifically binds human CD47), with at least one other agent (e.g., a cell death-inducing agent, e.g., an apoptosis inducing-agent, e.g., a Bcl-2 inhibitor), and includes staggered doses of these agents (e.g., time-staggered) and variations in dosage amounts. This includes one agent being administered before, overlapping with (partially or totally), or after administration of another agent. In some embodiments, a dosing strategy (e.g., a sequential dosage), creates a synergistic effect when both agents (e.g., an antibody that specifically binds human CD47 and a cell death-inducing agent, e.g., an apoptosis inducing agent) are administered to a subject such that the combined effects are greater than the additive effect of each agent when administered alone.

As used herein, the term “subject” includes any human or non-human animal. For example, the methods and compositions of the present invention can be used to treat a subject with an immune disorder. The term “non-human animal” includes all vertebrates, e.g., mammals and non-mammals, such as non-human primates, sheep, dog, cow, chickens, amphibians, reptiles, etc.

As used herein, the terms “synergy” and “synergistic” refer to the effect achieved when the active ingredients used together is greater than the sum of the effects that results from using the compounds separately. In some aspects, synergy results when the effect of the active ingredients used together, for example in an in vitro cell death assay as disclosed herein, is greater than the effects on cell death that result from either compound individually. In some aspects, synergy results in vitro when cell death is increased, enhanced or augmented when the active ingredients are used together relative to the effect on cell death of either compound individually. For example, in some aspects synergy results in vitro when cell death as determined by, for example, Annexin V/PI staining, is increased by at least 1-fold, at least 2-fold or more, when the active ingredients are used together relative to the effect of either compound individually on cell death. For example, in some aspects synergy results in vitro when cell death as determined by, for example, Annexin V/PI staining, is increased by at least 100%, at least 110%, at least 120%, at least 130%, at least 140%, at least 150% or more when the active ingredients are used together relative to the effect of either compound individually on cell death.

In some aspects, synergy results when a treatment outcome of the active ingredients used together is enhanced, augmented or improved over a treatment outcome of either compound individually. For example, in some aspects, synergy results in vivo when the effect of the active ingredients administered together (e.g., to a subject in need thereof as disclosed herein) provides an enhanced, augmented or improved treatment outcome in the subject, as compared to a treatment outcome when either compound is administered individually. For example, in some aspects synergy results in vivo when the effect of the active ingredients administered together inhibits, reduces or delays tumor growth or inhibits, reduces or delays tumor re-growth, or extends or prolongs survival of a subject to a greater extent than the effect of either compound individually on tumor growth, tumor re-growth or survival. For example, in some aspects, synergy results in vivo when a combined treatment with the active ingredients inhibits, reduces, or delays tumor growth or inhibits, reduces or delays tumor re-growth to a greater extent than the effect of either compound individually on tumor growth or tumor re-growth. For example, in some aspects synergy results in vivo when a combined treatment with the active ingredients inhibits, reduces, or delays tumor growth or inhibits, reduces, or delays tumor re-growth by at least 10%, at least 20%, at least 30%, at least 40%, at least 50%, at least 60%, at least 70%, at least 80%, at least 90%, or at least 100% or more than the effect of either compound individually on tumor growth or tumor re-growth. In some aspects, synergy results in vivo when a combined treatment with the active ingredients extends or prolongs survival in a subject to a greater extent than the survival which results from treatment with either compound individually. For example, in some aspects, synergy results in vivo when a combined treatment with the active ingredients extends, or prolongs the survival in a subject by at least 5 days, at least 10 days, at least 20 days, at least 30 days, at least 40 days, at least 50 days or more than the survival which results from treatment with either compound individually.

In some aspects, the combination of an anti-CD47 antibody as described herein and an apoptosis inducing agent (e.g., a BCL-2 inhibitor) results in a synergistic effect on the induction of cell death (e.g., apoptosis) and/or on the induction, promotion, or enhancement of an anti-tumor immune response. In some aspects, synergy results when a combination of an anti-CD47 antibody and an apoptosis inducing agent induces, promotes, enhances, or augments cell death of a tumor cell and/or induces, promotes, enhances, or augments an anti-tumor immune response that is promoted, enhanced, augmented, or improved relative to the cell death or anti-tumor immune response of either compound individually. For example, in some aspects, synergy results when the combination of an anti-CD47 antibody and an apoptosis inducing agent induces, promotes, enhances, or augments cell death or induces, promotes, enhances, or augments an anti-tumor immune response to a greater extent than the a treatment outcome (e.g., inhibition of tumor growth or tumor re-growth, reduction in tumor burden, prolonged survival) of either compound individually.

In some aspects, the combination of an anti-CD47 antibody as disclosed herein and an ICD inducing agent results in a synergistic effect on the induction of cell death (e.g., ICD) and/or a synergistic effect on the induction, promotion, or enhancement of an anti-tumor immune response. In some aspects, synergy results when a combination of an anti-CD47 antibody and an ICD inducing agent induces, promotes, or enhances ICD of a tumor cell and/or induces, promotes or enhances an anti-tumor immune response that is enhanced or improved relative to the cell death or an anti-tumor immune response of either compound individually. For example, in some aspects, synergy results when the combination of an anti-CD47 antibody and an ICD inducing agent induces, promotes, or enhances cell death or induces, promotes or enhances an anti-tumor immune response to a greater extent than a treatment outcome (e.g., inhibition of tumor growth or tumor re-growth, reduction in tumor burden, prolonged survival) of either compound individually.

In some embodiments, a synergistic effect is attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect is attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

As used herein, the term “targeted therapeutic agent” (alternatively “targeted agent”) refers to a chemical, biological, or pharmacological agent useful in the treatment of cancer that specifically and/or selectively targets cellular components necessary for the formation, development, and/or maintenance of cancer cells and excludes those pharmacological agents (e.g., chemotherapeutic agents) that are generally cytotoxic, non-specific intracellular poisons, such as those that function to inhibit the process of cell division known as mitosis. Targeted therapeutic agents can induce one or more cell death modalities, including apoptosis-induced cell death.

As used herein, the terms “therapeutically effective amount” or “therapeutically effective dose,” or similar terms used herein are intended to mean an amount of an agent (e.g., an antibody that specifically binds human CD47 or a cell death-inducing agent) that will elicit the desired biological or medical response (e.g., an improvement in one or more symptoms of a cancer).

The terms “treat,” “treating,” and “treatment,” as used herein, refer to therapeutic or preventative measures described herein. The methods of “treatment” employ administration to a subject, in need of such treatment, a human antibody and/or antagonist of the present disclosure, for example, a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

As used herein, the term “unrearranged” or “germline configuration” refers to a V segment refers to the configuration wherein the V segment is not recombined so as to be immediately adjacent to a D or J segment.

As used herein, the term “vector” is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, the term “VH” means a variable heavy domain.

As used herein, the term “VL” means a variable light domain.

Reference to particular amino acids may be made in respect of common 1-letter or 3-letter codes as commonly understood by persons skilled in the art. Any reference to an “X” amino acid is reference to a variable amino acid. Amino acids can be modified as described herein.

Antibodies that Specifically Bind Human CD47

The present disclosure provides antibodies that specifically bind human CD47, or antigen binding fragments thereof. CD47, also known as integrin-associated protein (TAP), ovarian cancer antigen OA3, Rh-related antigen and MERG, is a multi-spanning transmembrane receptor belonging to the immunoglobulin superfamily. CD47 expression and/or activity has been implicated in a number of diseases and disorders, e.g., cancer. CD47 interacts with SIRPα (signal-regulatory-protein a) on macrophages and thereby inhibits phagocytosis. An amino acid sequence of an exemplary human CD47 protein is provided in SEQ ID NO: 1 (NCBI Reference Sequence: NP 001768.1). An mRNA sequence encoding an exemplary human CD47 protein is provided in SEQ ID NO: 2 (NCBI Reference Sequence: NP 001768.1).

In some embodiments, the anti-CD47 antibody is capable of inhibiting, interfering with or blocking the interaction between CD47 and its cognate SIRPα ligand, without causing significant, or detectable, hemagglutination of erythrocytes, e.g., human erythrocytes. In some embodiments, the antibody causes less hemagglutination of human erythrocytes than a reference anti-CD47 antibody, or causes less than 90%, 80%, 70%, 60%, 50%, 40%, 30%, 20%, or 10% or less hemagglutination of human erythrocytes relative to a reference anti-CD47 antibody. Exemplary reference anti-CD47 antibodies include, but are not limited to, B6H12, MABL, BRIC126, and CC2C6. In some embodiments, the antibody causes substantially no hemagglutination of erythrocytes, e.g., human erythrocytes, for example, the antibody causes less than 50%, 40%, 30%, 20%, or 10% or less hemagglutination of human erythrocytes than a reference anti-CD47 antibody, such as B6H12, MABL, BRIC126, and CC2C6, when tested under the same or similar conditions.

In some embodiments, the anti-CD47 antibody causes a potent blocking of the interaction between CD47 and SIRPα without causing a significant level of hemagglutination of erythrocytes. For example, the anti-CD47 antibody blocks at least 40%, at least 50%, at least 55%, at least 60%, at least 65%, at least 70%, at least 75%, at least 80%, at least 85%, at least 90%, at least 95%, or at least 99% of the interaction between CD47 and SIRPα as compared to the level of interaction between CD47 and SIRPα in the absence of the anti-CD47 antibody. Optionally, the anti-CD47 antibody also causes less hemagglutination of human erythrocytes than a reference anti-CD47 antibody, or causes less than 90%, 80%, 70%, 60%, 50%, 40%, 30%, 20%, or 10% or less hemagglutination of human erythrocytes relative to a reference anti-CD47 antibody. Exemplary reference antibodies include B6H12, MABL, BRIC126, and CC2C6.

In some embodiments, the anti-CD47 antibody does not induce phagocytosis of red blood cells to a significant or detectable level. In some embodiments, the anti-CD47 antibody has reduced (e.g., 10%, 20%, 30%, 40%, 50%, 60%, 70%, 80%, or 90% reduced) phagocytic activity towards red blood cells relative to a reference anti-CD47 antibody. Exemplary reference antibodies include B6H12, MABL, BRIC126, and CC2C6.

In certain embodiments, the anti-CD47 antibody enhances macrophage activity. For example, the antibody enhances the phagocytic activity of a macrophage, e.g., an unpolarized macrophage, or an M1 or M2 polarized macrophage. In certain embodiment, the phagocytic activity is enhanced, e.g., 1%, 5%, 10%, 20%, 30%, 40%, 50%, 60%, 70%, 80%, or 90%, relative to a macrophage in the absence of the anti-CD47 antibody.

In some embodiments, the anti-CD47 antibody enhances macrophage activity. For example, the antibody enhances the phagocytic activity of a macrophage, e.g., an unpolarized macrophage, or an M1 or M2 polarized macrophage. In some embodiments, the phagocytic activity is enhanced, e.g., 1%, 5%, 10%, 20%, 30%, 40%, 5 50%, 60%, 70%, 80%, or 90%, relative to a macrophage in the absence of the anti-CD47 antibody.

In some embodiments, the anti-CD47 antibody enhances macrophage phagocytic activity towards a cancer cell, e.g., an AML cell. In some embodiments, the phagocytic activity is enhanced, e.g., 1%, 5%, 10%, 20%, 30%, 40%, 50%, 60%, 70%, 80%, or 90%, relative to a macrophage in the absence of the anti-CD47 antibody.

In some embodiments, the anti-CD47 antibody binds CD47 with a K_{D} of 20 nM, 15 nM, 10 nM, 9 nM, 8 nM, 7 nM, 6 nM, 5 nM, 4 nM, 3 nM, 2 nM, 1 nM or lower, as measured using standard binding assays, for example, surface plasmon resonance or bio-layer interferometry. In some embodiments, the anti-CD47 antibody binds CD47 with a K_{D} of 1 nM or less.

Anti-CD47 antibodies may be characterized relative to a reference anti-CD47 antibody, for example, B6H12, 2D3, MABL, CC2C6, or BRIC126. Antibody B6H12 is described, for example, in U.S. Pat. Nos. 5,057,604 and 9,017,675, and is commercially available from Abcam, PLC, Santa Cruz Biotechnology, Inc., and eBioscience, Inc. Antibody MABL is described, for example, in Uno S, Kinoshita Y, Azuma Y et al. (2007) ONCOL. REP. 17: 1189-94, and Kikuchi Y, Uno S, Yoshimura Y et al. (2004) BIOCHEM. BIOPHYS. RES. COMMUN. 315: 912-8. Antibody CC2C6 is described, for example, in Martina Seiffert et al. (1997) BLOOD 94(11): 3633-3643, and is commercially available from Santa Cruz Biotechnology, Inc. Antibody BRIC126 is described, for example, in Avent et al. (1988) BIOCHEM. J. 251: 499-505. Antibody 2D3 is commercially available from eBioscience, Inc., and unlike the other reference antibodies, does not interfere with the binding between CD47 and SIRPα.

In some embodiments, the anti-CD47 antibody comprises: (a) an immunoglobulin heavy chain variable region comprising the structure HCDR1-HCDR2-HCDR3 and (b) an immunoglobulin light chain variable region, wherein the heavy chain variable region and the light chain variable region together define a single binding site for binding CD47. In some embodiments, the HCDR1 comprises the amino acid sequence set forth in SEQ ID NO: 5; the HCDR2 comprises the amino acid sequence set forth in SEQ ID NO: 6; and the HCD3 comprises the amino acid sequence set forth in SEQ ID NO: 7. The HCDR1, HCDR2, and HCDR3 sequences are interposed between immunoglobulin FR sequences.

In some embodiments, the anti-CD47 antibody comprises: (a) an immunoglobulin light chain variable region comprising the structure LCDR1-LCDR2-LCDR3, and (b) an immunoglobulin heavy chain variable region, wherein the light chain variable region and the heavy chain variable region together define a single binding site for binding CD47. In some embodiments, the LCDR1 comprises the amino acid sequence set forth in SEQ ID NO: 8; the LCDR2 comprises the amino acid sequence set forth in SEQ ID NO: 9; and the LCDR3 comprises the amino acid sequence set forth in SEQ ID NO: 10. The LCDR1, LCDR2, and LCDR3 sequences are interposed between immunoglobulin FR sequences.

In certain embodiments, the anti-CD47 antibody comprises: (a) an immunoglobulin heavy chain variable region comprising the structure HCDR1-HCDR2-HCDR3 and (b) an immunoglobulin light chain variable region comprising the structure LCDR1-LCDR2-LCDR3, wherein the heavy chain variable region and the light chain variable region together define a single binding site for binding CD47. In some embodiments, the HCDR1 comprises the amino acid sequence set forth in SEQ ID NO: 5; the HCDR2 comprises the amino acid sequence set forth in SEQ ID NO: 6; and the HCDR3 comprises the amino acid sequence set forth in SEQ ID NO: 7. The HCDR1, HCDR2, and HCDR3 sequences are interposed between immunoglobulin FR sequences. In some embodiments, the LCDR1 comprises the amino acid sequence set forth in SEQ ID NO: 8; the LCDR2 comprises the amino acid sequence set forth in SEQ ID NO: 9; and the LCDR3 comprises the amino acid sequence set forth in SEQ ID NO: 10. The LCDR1, LCDR2, and LCDR3 sequences are interposed between immunoglobulin FR sequences.

In some embodiments, the anti-CD47 antibody comprises an immunoglobulin heavy chain variable region (VH) comprising the amino acid sequence set forth in SEQ ID NO: 3, and an immunoglobulin light chain variable region (VL). In some embodiments, the anti-CD47 antibody comprises an immunoglobulin light chain variable region (VL) comprising the amino acid sequence set forth in SEQ ID NO: 4, and an immunoglobulin heavy chain variable region (VH). In certain embodiments, the anti-CD47 antibody comprises an immunoglobulin heavy chain variable region (VH) comprising the amino acid sequence set forth in SEQ ID NO: 3, and an immunoglobulin light chain variable region (VL) comprising the amino acid sequence set forth in SEQ ID NO: 4.

In some embodiments, the anti-CD47 antibody comprises an immunoglobulin heavy chain variable region (VH) comprising an amino acid sequence that is at least 70%, 75%, 80%, 85%, 90%, 95%, 96%, 97%, 98%, or 99% identical to the amino acid sequence set forth in SEQ ID NO: 3 and comprises an immunoglobulin light chain variable region (VL) comprising an amino acid sequence that is at least 70%, 75%, 80%, 85%, 90%, 95%, 96%, 97%, 98%, or 99% identical to the amino acid sequence set forth in SEQ ID NO: 4.

In some embodiments, the anti-CD47 antibody comprises the antibodies described in U.S. Pat. No. 9,650,441 (incorporated herein by reference), including, for example, the antibody referred to as 2.3D11. Antibody 2.3D11 was produced by immunizing mice carrying a human immunoglobulin immune repertoire in place of the murine repertoire with soluble CD47-Fc fusion protein. Hybridomas expressing anti-CD47 monoclonal antibodies, including antibody 2.3D11, were isolated. The isolated hybridomas expressed antibodies having both heavy and light chains with fully human variable domains and rat constant domains. Once isolated, the constant regions of the heavy chain were replaced with heavy chain constant regions from human IgG1, human IgG4, or human IgG4 containing Ser228Pro and Leu235Glu substitutions. U.S. Pat. No. 9,650,441 characterizes antibody 2.3D11 and demonstrates that this antibody can potently inhibit the interaction between CD47 and SIRPα, and enhance phagocytosis of tumor cells.

In some embodiments, the anti-CD47 antibody comprises the antibodies described in U.S. Pat. No. 9,045,541, including, for example, the antibodies referred to as 2A1, 2A1-xi, AB6.12, AB6.12-IgG1, AB6.12-IgG4P and AB6.12-IgG4PE. For example antibody AB6.12 comprises the variable heavy chain sequence of SEQ ID NO: 11 and the variable light chain sequence of SEQ ID NO: 42 as set forth in Table 1 of U.S. Pat. No. 9,045,541 (incorporated herein by reference). In some embodiments, the anti-CD47 antibody comprises a chimeric or humanized version of the monoclonal antibody 5F9G4 described in Liu et al. (2016) PLoS ONE 10(9):e0137345. Chimeric and humanized versions of 5F9G4, such as humanized 5F9G4 (hu5F9G4)) are disclosed in U.S. Pat. No. 9,017,675, incorporated herein by reference. In some embodiments, the anti-CD47 antibody comprises the variable heavy and light chain sequences of antibody CC-90002 (Celgene). In some embodiments, the anti-CD47 antibody comprises the variable heavy and light chain sequences of antibody TI-061 (Tioma Therapeutics). In some embodiments, the agent that specifically binds CD47, wherein the agent comprises an antibody or polypeptide comprising an Fc domain, comprises the variable heavy and light chain sequences of antibody INBRX-103 (CC-90002) (Inhibrx LP).

In some embodiments, it is contemplated that a heavy chain variable region sequence, for example, the VH sequence set forth in SEQ ID NO: 3, may be covalently linked to a variety of heavy chain constant region sequences known in the art. Similarly, it is contemplated that a light chain variable region sequence, for example, the VL set forth in SEQ ID NO: 4, may be covalently linked to a variety of light chain constant region sequences known in the art. Similarly, it is contemplated that the heavy chain variable region sequences and/or the light chain variable region sequences of the anti-CD47 antibodies described in U.S. Pat. No. 9,045,541 and Liu et al. (2016) supra may be linked to a variety of heavy constant region sequences and/or light chain constant region sequences known in the art.

For example, the antibody may have a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has effector function and can fix complement. In other embodiments the antibody does not recruit effector cells or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In certain embodiments, the constant region of the heavy chain of the antibody is a human IgG1 isotype, having an amino acid sequence set forth in SEQ ID NO: 18. In certain embodiments, the human IgG1 constant region is modified at amino acid Asn297 to prevent to glycosylation of the antibody, for example Asn297Ala (N297A). In certain embodiments, the constant region of the antibody is modified at amino acid Leu235 to alter Fc receptor interactions, for example Leu235Glu (L235E) or Leu235Ala (L235A). In certain embodiments, the constant region of the antibody is modified at amino acid Leu234 to alter Fc receptor interactions, e.g., Leu234Ala (L234A). In certain embodiments, the constant region of the antibody is modified at amino acid Glu233, e.g., Glu233Pro (E233P). In some embodiments, the constant region of the antibody is altered at both amino acid 234 and 235, for example Leu234Ala and Leu235Ala (L234A/L235A). In certain embodiments, the constant region of the antibody is altered at amino acids 233, 234, and 234, for example, Glu233Pro, Leu234Ala, and Leu235Ala (E233P L234A/L235A) (Armour KL. et al. (1999) Eur. J. Immunol. 29(8):2613-24). All residue numbers are according to EU numbering (Kabat, E. A., et al., supra).

In certain embodiments, the constant region of the heavy chain of the antibody is a human IgG2 isotype, having an amino acid sequence set forth in SEQ ID NO: 19. In certain embodiments, the human IgG2 constant region is modified at amino acid Asn297 to prevent to glycosylation of the antibody, e.g., Asn297Ala (N297A), where the residue numbers are according to EU numbering (Kabat, E. A., et al., supra).

In certain embodiments, the constant region of the heavy chain of the antibody is an human IgG3 isotype, having an amino acid sequence set forth in SEQ ID NO: 20. In certain embodiments, the human IgG3 constant region is modified at amino add Asn297 to prevent to glycosylation of the antibody, e.g., Asn297Ala (N297A.). In some embodiments, the human IgG3 constant region is modified at amino acid. Arg435 to extend the half-life, e.g., Arg435H (R435H). All residue numbers are according to EU numbering (Kabat, E. A., et al., supra).

In certain embodiments, the constant region of the heavy chain of the antibody is an human IgG4 isotype, having an amino acid sequence set forth in SEQ ID NO: 21. In certain embodiments, the human IgG4 constant region is modified within the hinge region to prevent or reduce strand exchange, e.g., in some embodiments human IgG4 constant region is modified at Ser228, e.g., Ser228Pro (S228P). In other embodiments, the human IgG4 constant region is modified at amino acid Leu235 to alter Fc receptor interactions, e.g., Leu235Glu (L235E). In some embodiments, the human IgG4 constant region is modified at both Ser22S and. Leu335, e.g., Ser228Pro and Leu235Glu (S228P/L235E), and comprises the amino acid sequence of SEQ ID NO: 21, hereafter referred to as IgG4mt2. In some embodiments, the human IgG4 constant region is modified at amino acid Asn297 to prevent to glycosylation of the antibody, e.g., Asn297Ala (N297A). All residue numbers are according to EU numbering (Kabat, E. A., et al., supra).

In certain embodiments, the constant region of the heavy chain of the antibody is a human IgM isotype.

In certain embodiments, the human IgG constant region is modified to enhance Fc receptor binding. Examples of Fc mutations that enhance binding to Fc receptors are Met252Tyr, Ser254Thr, Thr256Glu (M252Y, S254T, T256E, respectively) (Dall'Acqua et al. (2006) J. BIOL. CHEM. 281(33): 23514-23524), or Met428Leu and Asn434Ser (M428L, N434S) (Zalevsky et al. (2010) NATURE BIOTECH. 28(2): 157-159). All residue numbers are according to EU numbering (Kabat, E. A., et al., supra).

In some embodiments, the human IgG constant region is modified to alter antibody-dependent cellular cytotoxicity (ADCC) and/or complement-dependent cytotoxicity (CDC), e.g., the amino acid modifications described in Natsume et al, (2008) CANCER RES. 68(10): 3863-72; Idusogie et al. (2001) J. IMMUNOL. 166(4): 2571-5; Moore et al. (2010) MABS 2(2): 181-189; Lazar et al. (2006) PROC. NAIL. ACAD. SCI. USA 103(11): 4005-4010, Shields et al. (2001) J. BIOL. CHEM. 276(9): 6591-6604; Stavenhagen et al. (2007) CANCER RES. 67(18): 8882-8890; Stavenhagen et al. (2008) ADVAN. ENZYME REGUL. 48: 152-164; Alegre et al. (1992) J. IMMUNOL. 148: 3461-3468.

In some embodiments, the human IgG constant region is modified to induce heterodimerization. For example, a heavy chain having an amino acid modification within the CH3 domain at Thr366, e.g., a substitution with a more bulky amino acid, e.g., Try (T366W), is able to preferentially pair with a second heavy chain having a CH3 domain having amino acid modifications to less bulky amino acids at positions Thr366, Leu368, and Tyr407, Ser; Ala and Val, respectively (T366S/L368A/Y407V). Heterodimerization via CH3 modifications can be further stabilized by the introduction of a disulfide bond, for example by changing Ser354 to Cys (S354C) and Y349 to Cys (Y349C) on opposite CH3 domains (see, Carter (2001) J. IMMUNOL. METHODS 248: 7-15).

Accordingly, in some embodiments, the anti-CD47 antibody comprises an immunoglobulin heavy chain comprising an amino acid sequence selected from SEQ ID NO: 12, SEQ ID NO: 13 and SEQ ID NO: 14, and an immunoglobulin light chain. In some embodiments, the anti-CD47 antibody comprises an immunoglobulin light chain comprising an amino acid sequence of SEQ ID NO: 16, and an immunoglobulin heavy chain. In some embodiments, the anti-CD47 antibody comprises an immunoglobulin heavy chain comprising an amino acid sequence selected from SEQ ID NO: 12, SEQ ID NO: 13, and SEQ ID NO: 14, and an immunoglobulin light chain comprising the amino acid sequence of SEQ ID NO: 16.

Exemplary nucleotide sequences encoding anti-CD47 antibodies disclosed herein include the nucleotide sequence provided in SEQ ID NO: 25, encoding an immunoglobulin heavy chain comprising a heavy chain variable domain of the 2.3D11 antibody and a human IgG1 heavy chain constant domain (corresponding to the amino acid sequence provided in SEQ ID NO: 12), the nucleotide sequence provided in SEQ ID NO: 26, encoding an immunoglobulin heavy chain comprising a heavy chain variable domain of the 2.3D11 antibody and a human IgG4 heavy chain constant domain (corresponding to the amino acid sequence provided in SEQ ID NO: 13), the nucleotide sequence provided in SEQ ID NO: 27, encoding an immunoglobulin heavy chain comprising a heavy chain variable domain of the 2.3D11 antibody and a human IgG4 heavy chain constant domain with Ser228Pro and Leu235Glu substitutions (corresponding to the amino acid sequence provided in SEQ ID NO: 14), and the nucleotide sequence provided in SEQ ID NO: 28, encoding an immunoglobulin light chain comprising a light chain variable domain of the 2.3D11 antibody and a human kappa constant domain (corresponding to the amino acid sequence provided in SEQ ID NO: 16).

In some embodiments, the anti-CD47 antibody binds to the same epitope present in CD47 as that bound by a disclosed antibody, e.g., the 2.3D11 antibody comprising an immunoglobulin heavy chain variable region referred to herein as 2.3D11-V_{H} and an immunoglobulin light chain variable region referred to herein as 2.3D11-V_{L}. In some embodiments, the invention provides antibodies that compete for binding to CD47 with a disclosed antibody, e.g., the 2.3D11 antibody comprising an immunoglobulin heavy chain variable region referred to herein as 2.3D11-V_{H} and an immunoglobulin light chain variable region referred to herein as 2.3D11-V_{L}.

Competition assays for determining whether an antibody binds to the same epitope as, or competes for binding with a disclosed antibody, e.g., the 2.3D11 antibody, are known in the art. Exemplary competition assays include immunoassays (e.g., ELISA assays, RIA assays), surface plasmon resonance, (e.g., BIAcore analysis), bio-layer interferometry, and flow cytometry.

Typically, a competition assay involves the use of an antigen (e.g., a human CD47 protein or fragment thereof) bound to a solid surface or expressed on a cell surface, a test CD47-binding antibody and a reference antibody (e.g., the 2.3D11 antibody). In some embodiments, the reference antibody is labeled and the test antibody is unlabeled. In some embodiments, the reference antibody is unlabeled and the test antibody is labeled. Competitive inhibition is measured by determining the amount of labeled reference antibody bound to the solid surface or cells in the presence of the test antibody. Usually the test antibody is present in excess (e.g., 1×, 5×, 10×, 20×or 100×). Antibodies identified by competition assay (i.e., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20×or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In some embodiments, isolated human antibodies contain one or more somatic mutations. In these cases, antibodies can be modified to a human germline sequence to optimize the antibody (i.e., a process referred to as germlining).

Generally, an optimized antibody has at least the same, or substantially the same, affinity for the antigen as the non-optimized (or parental) antibody from which it was derived. Preferably, an optimized antibody has a higher affinity for the antigen when compared to the parental antibody.

The antibody can be conjugated to an effector moiety such as a small molecule toxin or a radionuclide using standard in vitro conjugation chemistries. If the effector moiety is a polypeptide, the antibody can be chemically conjugated to the effector or joined to the effector as a fusion protein. Construction of fusion proteins is within ordinary skill in the art.

### Cell Death-Inducing Agents

In some aspects, the present disclosure provides combination of a monoclonal antibody that specifically binds human CD47, or an antigen binding fragment thereof, as described herein, and a cell death-inducing agent. In some embodiments, the cell death-inducing agent induces one or more cell death modalities. In one embodiment, the cell death modality is immunogenic cell death. In one embodiment, the cell death modality is apoptosis. In one embodiment, the cell death-inducing agent induces the intrinsic apoptotic pathway.

In some embodiments, the cell-death inducing agent is a targeted therapeutic agent or a targeted therapeutic. In some embodiments, the targeted therapeutic agent induces apoptosis. In some embodiments, the targeted therapeutic agent induces the intrinsic apoptotic pathway. In some embodiments, the targeted therapeutic agent inhibits a DNA damage response pathway. In some embodiments, the targeted therapeutic agent is a kinase inhibitor. In some embodiments, the targeted therapeutic agent is a proteasome inhibitor.

In some embodiments, the cell-death inducing agent is a cytotoxic chemotherapeutic agent. In some embodiments, the cytotoxic chemotherapeutic agent induces immunogenic cell death.

Many cell death inducing agents, for example, the BCL-2 inhibitor ABT-199, induce cell death in a caspase-dependent manner. Therefore, without being bound by theory, it is believed that the combination of an anti-CD47 antibody (e.g., 2.3D11 having the heavy and light chain amino acid sequences set forth in SEQ ID Nos 13 and 16, respectively) and other cell death inducing agents (e.g., ABT-199) results in enhanced cell death because the anti-CD47 antibody (e.g., 2.3D11) induces cell death via a non-overlapping mechanism.

### Apoptosis-Inducing Agents

In some embodiments, the cell death-inducing agent used in combination with the anti-CD47 antibody is an agent that induces apoptosis of a cell of interest (e.g., cancer cell). The two main pathways of apoptosis are the intrinsic apoptotic pathway and the extrinsic apoptotic pathway. A third apoptotic pathway mediated by cytotoxic T cells is the perforin/granzyme apoptotic pathway. Each pathway requires specific triggering signals to begin an energy-dependent cascade of molecular events that culminate in a programmed cell death phenotype. Each pathway activates its own initiator caspase which in turn will activate the executioner caspase-3. The convergent execution pathway results in characteristic cytomorphological features including cell shrinkage, chromatin condensation, formation of cytoplasmic blebs and apoptotic bodies and finally phagocytosis of the apoptotic bodies by adjacent parenchymal cells, neoplastic cells or macrophages (Elmore (2007) Toxicol Pathol 35(4):495-516). In some embodiments, the apoptosis-inducing agent induces the intrinsic apoptotic pathway in the cell of interest (e.g., cancer cell).

Overexpression of anti-apoptotic genes can, in part, result in the resistance or absence of apoptosis (alternatively known as programmed cell death), a phenotype considered to be a hallmark of cancer (Hanahan and Weinberg (2000) Cell 100(1):57-70). Evasion of apoptosis by tumor cells is often mediated by upregulation of pro-survival and/or anti-apoptotic members of the Bcl-2 protein family, especially Bcl-2, Bcl-XL and myeloid cell leukemia 1 (MCL1), which control the intrinsic apoptotic pathway (Yip and Reed (2008) Oncogene 27:6398-6406; Dai et al, (2016) Cancer Transl Med 2:7-20). When expressed at elevated levels relative to pro-apoptotic effectors, these anti-apoptotic/pro-survival proteins inhibit cancer cells from initiating apoptosis. Upregulation of anti-apoptotic/pro-survival proteins can also inhibit inherent tumor-suppressing pathways as well as the effects extrinsic therapeutic treatments aimed at controlling or treating various malignancies. Pharmacological inhibition of the activities of Bcl-2 protein family members can directly induce apoptosis and can be useful for the treatment of cancer and other diseases (Bodur and Basaga (2012) Curr Med Chem 19(12):1804-1820).

The Bcl-2 family is dysregulated in acute myeloid leukemia (AML) and in B cell malignancies, including multiple myeloma (MM), with increases in the anti-apoptotic family members Bcl-2, BCL-XL and Mcl-1 known to occur. (Lauria F, et al., Leukemia 1997; 11: 2075-2078; Davids M S, et al., J Clin Oncol 2012; 30: 3127-3135). These anti-apoptotic family members have some redundancy and can help protect cells from apoptosis in overlapping, yet distinct fashions. MCL-1 (and BCL-X1) is an anti-apoptotic protein that antagonizes the pro-apoptotic effects of Bcl2 inhibitors such as ABT-199. ABT-199 disrupts association of Bcl2 with the pro-apoptotic molecule Bim, which can lead to apoptosis, but MCL-1 is able to sequester Bim and thereby prevent apoptosis. (Niu X, et al. Clin. Cancer Res. 2016; 22: 4440-4451). Thus, cancers often have more than one mechanism of resistance to cell death induction and combination of multiple targeted inhibitors can show synergistic induction of cell death in these cases. (Luedtke et al. Signal Transduction and Targeted Therapy 2017; 2: 17012).

ABT-199 is used in combination with cytotoxic agents, further supporting the theory that the combination of ABT-199 with a cell-death inducing agent provides benefit to patients. Without being bound by theory, it is believed that there is a two signal model where (1) conventional chemotherapy provides the overt cytotoxic signal and (2) ABT-199 takes the brakes off of the regulation of apoptosis. Without being bound by theory, it is believed that the mechanism of cell death induction via CD47 could act as this ‘first signal’ and replace or enhance the need for cytotoxics in combination with ABT-199.

Further, without being bound by theory, it is believed that the combination of ABT-199 with an anti-CD47 antibody (for example, 2.3D11 having the heavy and light chain sequences set forth in SEQ ID Nos 13 and 16) in a cell line with reduced ABT-199 sensitivity provides anti-tumor benefit to patients, and the combination of and anti-CD47 antibody (for example, 2.3D11 having the heavy and light chain sequences set forth in SEQ ID Nos 13 and 16) with inhibitors of other Bcl2 family members, such as MCL-1, may also lead to improved outcomes. For example, the MOLM-13 tumor cell line is a human AML cell line that expresses high levels of MCL-1 and has reduced sensitivity to ABT-199.

Accordingly, in some embodiments, the apoptosis-inducing agent is an inhibitor of the Bcl-2 protein family. Exemplary Bcl-2 inhibitors are described in U.S. Pat. Nos. 8,546,399 B2 and 9,174,982 B2, which are incorporated herein by reference in its entirety. The Bcl-2 inhibitor ABT-199 (alternatively Venetoclax, RG7601, GDC-0199) is a selective, oral Bcl-2 inhibitor in clinical development (Cang et al., (2015) J Hematol Oncol (2015) 8:129). Other exemplary Bcl-2 inhibitors include, but are not limited to, ABT-263 (alternatively known as Navitoclax) and GX15-070 (alternatively known as Obatoclax). In some embodiments, the cell death-inducing agent is an apoptosis inducing agent, wherein the apoptosis inducing agent is a Bcl-2 inhibitor such as ABT-199 (alternatively known as Venetoclax), ABT-263 (alternatively known as Navitoclax), GX15-070 (alternatively known as Obatoclax), or combinations thereof. In some embodiments, the cell death-inducing agent is ABT-199. In some embodiments, the cell death-inducing agent is ABT-263. In some embodiments, the cell death-inducing agent is GX15-070.

In some embodiments, the cell death-inducing agent is an apoptosis-inducing agent, wherein the apoptosis-inducing agent is an Mcl-1 inhibitor. Myeloid cell leukemia 1 (Mcl-1), a pro-survival member of the Bcl-2 family of proteins, is overexpressed and the Mcl-1 gene is amplified in many tumor types. Moreover, the overexpression of Mcl-1 is the cause of resistance to several chemotherapeutic agents (Belmar and Fesik (2015) Pharmacol Ther 145:76-84). Exemplary Mcl-1 inhibitors are described in PCT US 2015/047472, which is incorporated herein by reference in its entirety, in PCT US 2013/023205, which is incorporated herein by reference in its entirety, and in Belmar and Fesik (2015) Pharmacol Ther 145:76-84, which is incorporated herein by reference in its entirety. AMG176 is an inhibitor of MCL-1 (https://clinicaltrials.gov/ct2/show/NCT02675452). Upon administration, AMG176 binds to and inhibits the activity of Mcl-1. This disrupts the formation of Mcl-1/Bcl-2-like protein 11 (BCL2L11; BIM) complexes and induces apoptosis in tumor cells. In some embodiments, the Mcl-1 inhibitor is AMG176.

In some embodiments, the cell death-inducing agent is an apoptosis-inducing agent, wherein the apoptosis-inducing agent is a BCL-XL inhibitor. BCL-XL is an important factor for tumor development. Previous studies have shown that the BCL2 L1 locus, which encodes BCL-XL, is amplified in a variety of solid tumors (Beroukhim et al., (2010) Nature 463(7283):899-905. Exemplary BCL-XL inhibitors are described Opferman (2016) FEBSJ 283(14):2661-2675, which is incorporated herein by reference in its entirety. WEHI-539 is a selective BCL-XL small molecule inhibitor (Lessene et al., (2013) Nat Chem Biol 9(6):390-397). In some embodiments, the BCL-XL inhibitor is WEHI-539.

In some embodiments, the cell death-inducing agent is an apoptosis-inducing agent, wherein the apoptosis inducing agent is a MDM2 inhibitor. The mouse double minute 2 homolog (MDM2) protein, also known as E3 ubiquitin-protein ligase Mdm2, is an oncogenic protein and functions as the principal negative regulator of the p53 tumor suppresser gene. Overexpression of MDM2 serves as a molecular mechanism by which a cell can inactivate p53 to transform a normal cell into a cancer cell (Shi and Gu (2012) Genes Cancer 3(3-4):240-248). Loss of the MDM2 gene in mice can induce the p53-dependent apoptotic pathway in vivo (Rozieres et al., (2000) Oncogene 19:1691-1697). Pharmacological inhibition of the MDM2-p53 complex was found to induce apoptosis in p53 wild-type myeloma models in association with activation of a p53-mediated cell death program (Gu et al., (2014) PLoS ONE 9(9):e103015). AMG-232 is a selective small molecule inhibitor of the MDM2-p53 interaction (Sun et al., (2014) J Med Chem 57(4):1454-1472). In some embodiments, the MDM2 inhibitor is AMG-232.

### DNA Damage Response (DDR) Pathway Inhibiting Agents

In some embodiments, the cell death-inducing agent used in combination with the anti-CD47 antibody is an inhibitor of the DNA damage response (DDR) pathway. Defects in the DDR pathway or deficiencies in cellular DNA repair mechanisms can result in genomic instability, leading to oncogenic transformation and tumor formation (Hanks et al., (2004) Nat Genet 36:1159-1161; Weaver et al., (2007) Cancer Cell 11(1):25-36; Shih et al., (2001) Cancer Res 61(3):818-822; Lengauer et al., (1998) Nature 396(6712):643-649). Genomic instability often results in the generation of mutations that dysregulate growth and promote tumor cell invasion and metastasis (Hanahan and Weinberg (2011) Cell 144(5):646-674). DNA repair defects can be exploited in cancer therapy. The same defects in DNA repair that produced the oncogenic mutations in the first place make replication of the tumor cell more dependent on complementary, alternative, and/or compensatory DNA repair mechanisms to compensate for the oncogenic loss of the original DNA repair component. Because excessive genomic instability itself can induce cell death by inducing deadly mutations, mitotic catastrophe, or chromothripsis (Forment et al., (2012) Nat Rev Cancer 12(10):663-670; Rode et al., (2016) Int J Cancer 138(10):2322-2333), these complementary, alternative, and/or compensatory DNA repair components can be targeted by inhibitors to promote tumor-specific cell killing. Inhibitors of the DDR have direct activity against tumor cells with a specific DDR defect and can increase the efficacy of DNA damaging chemotherapy and radiotherapy (Curtin (2013) Br J Pharmacol 169(8):1745-1765).

Agents that inhibit DNA repair and/or DNA response pathways include, but are not limited to, PARP inhibitors (e.g., Rucaparib (alternatively known as AG-014699 or CO-338), INO-1001, Olaparib (alternatively known as KU59436 or AZD2281), Niraparib, Veliparib (alternatively known as ABT-888), MK4827, CEP-9722, GPI21016/E7016, and BMN-673) (see e.g., Curin (2013) Br J Pharmacol 169(8):1745-1765, which is incorporated herein by reference in its entirety). PARP inhibitors can be used to sensitize tumors to other chemotherapeutic agents (e.g., DNA damaging agents, Temozolomide) to treat specific cancers (Cheng et al., (2005) Mol Cancer Ther 4(9):1364-1368). In some embodiments, the cell death-inducing agent is an agent that inhibits the DNA damage response pathway and/or DNA repair pathway in tumor cells.

In some embodiments, the inhibitor of DNA repair and/or DDR is a PARP inhibitor. In some embodiments, the PARP inhibitor is olapirib, niraparib, rucaparib, or combinations thereof. In some embodiments, the inhibitor of the DNA repair and/or DDR is temozolomide.

### Kinase Inhibitors

In some embodiments, the cell death-inducing agent used in combination with the anti-CD47 antibody, is a kinase inhibitor. Several kinases in the B cell receptor (BCR) pathway can be targeted with small molecules to effectively interrupt BCR signaling in vivo, resulting in the inhibition of activation, proliferation, and survival of tumor cells, particularly those of hematological malignancies. For example, recent reports of preclinical assessments of ibrutinib (alternatively known as PCI-32765) demonstrated inhibition of BCR but direct inhibitory effects in diffuse large B-cell lymphoma (DLBCL) and mantle cell lymphoma (MCL) cells and primary leukemia cells from patients with chronic lymphocytic leukemia (CLL) (Herman et al., (2010) Blood 116(12):2078-88). Kinases involved in B cell receptor signaling include, but are not limited to, Bruton's tyrosine kinase (BTK), phosphoinositide 3-kinase (PI3K), and spleen tyrosine kinase (SYK). Exemplary inhibitors of BTK and other kinases involved in BCR signaling include, but are not limited to, fostamatinib, entospletinib, idelalisib, duvelisib, AMG-319, TGR-1202, ibrutinib, CC-292, ONO-4059, and ACP-196) (see e.g., Wiestner (2015) Haematologica 100(12):1495-1507, which is incorporated herein by reference in its entirety).

In some embodiments, the kinase inhibitor inhibits BTK, PI3K, SYK, or any combination thereof. In some embodiments, the kinase inhibitor is ibrutinib.

### Proteasome Inhibitors

In some embodiments, the cell death-inducing agent used in combination with the anti-CD47 antibody, is a proteasome inhibitor. Proteasome inhibition has emerged as an effective therapeutic strategy for treating multiple myeloma (MM) and some lymphomas (Adams (2004) Nat Rev Cancer 4:349-360). In 2003, Bortezomib (BTZ) became the first proteasome inhibitor approved by the U.S. Food and Drug Administration (FDA) (Chen et al., (2011) Curr Cancer Drug Targets 11(3):239-253). Bortezomib or Velcade® is known to induce cell death by upregulation of NOXA, a pro-apoptotic BH3 protein and trigger cleavage of Mcl-1, caspase-3, caspase-9, and PARP. Bortezomib also induces ICD, leading to the release of DMAPs, including exposure of heat shock protein 90 (hsp90) on the surface of dying cells thereby providing activating signals to dendritic cells and inducing antitumor immunity (Spisek (2007) Blood 109: 4839-4845).

The ubiquitin-proteasome system (UPS) is essential for maintenance of cell homeostasis, in particular the balance of protein synthesis and degradation (Ciechanover (1994) Cell 79:13-21; Goldberg et al., (1997) Biol Chem 378:131-140). Evidence of dysregulation of protein degradation has been observed in multiple malignancies and has been shown to affect the cell growth, development and survival of tumors and tumor cells. Proteasomal mRNA levels are consistently markedly increased in a variety of malignant human hematopoietic cell lines compared with peripheral lymphocytes and monocytes from healthy adults (Kumatori et al., (1990) Proc Natl Acad Sci USA 87:7071-7075). Tumorigenic cells are more dependent upon proteasomal activity and thus more sensitive to inhibition. Proteasome inhibition can result in growth suppression and induction of apoptosis in tumor cells (Hideshima et al., (2009) Blood 114(5):1046-1052).

Exemplary proteasome inhibitors include, but are not limited to, bortezomib (alternatively known as Velcade® or PS-341, carfilzomib (alternatively known as Kyprolis® or PR171, ixazomib (alternatively known as MLN-9708/2238), delanzomib (alternatively known as CEP-18770), oprozomib (alternatively known as ONX-0912 or PR-047, and marizomib (alternatively known as NPI-0052 or salinosporamide A) (see e.g, Dou and Zonder (2014) Curr Cancer Drug Targets 14(6):517-5, which is incorporated herein by reference in its entirety). In some embodiments, the proteasome inhibitor is bortezomib.

### Chemotherapeutic Agents

In some embodiments, the cell death-inducing agent used in combination with an anti-CD47 antibody, is a chemotherapeutic agent. Exemplary chemotherapeutic agents that induce immunogenic cell death (ICD) include, but are not limited to, anthracyclines (e.g., doxorubicin, idarubicin, daunorubicin, cytarabine, epirubicin, valrubicin and mitoxantrone) (see e.g., Minotti et al., (2004) Pharmacol Rev 56(2):185-229), topoisomerase inhibitors (e.g., topotecan; Hycamtin, camptothecin, etoposide) (see e.g., Pommier et al., (2010) Chem Biol 17(5):421-433; which is incorporated herein by reference in its entirety), bleomycin (Kimura et al., (1972) Cancer 29(1):58-60), gemcitabine (Plunkett et al., (1995) Semin Oncol 22(4 Suppl 11):3-10), platins (e.g., carboplatin, cisplatin, oxaliplatin, satraplatin, picoplatin) (Kelland (2007) Nat Rev Cancer 7(8):573-584), taxanes (e.g., docetaxel, paclitaxel, abraxane) (Abal et al., (2003) Curr Cancer Drug Targets 3(3):193-203), DNA alkylating agents (eg. cyclophosphamide, bendamustine) (Leoni et al., (2008) Clin Cancer Res 14(1):309-317), CHOP (drug combination of cyclophosphamide, doxorubicin hydrochloride, vincristine and prednisone) (Dunleavy (2014) Hematology Am Soc Hematol Educ Program 2014(1):107-112), and fluorouracil and derivatives thereof (Alvarez et al., (2012) Expert Opin Ther Pat 22(2):107-123, which is incorporated herein by reference in its entirety).

Recent studies have demonstrated that therapeutic outcomes with specific chemotherapeutic agents (e.g. anthracyclines) correlate strongly with their ability to induce a process of immunogenic cell death (ICD) in cancer cells. This process generates a series of signals that stimulate the immune system to recognize and clear tumor cells. Extensive studies have revealed that chemotherapy-induced ICD occurs via the exposure/release of calreticulin (CALR), ATP, chemokine (C-X-C motif) ligand 10 (CXCL10) and high mobility group box 1 (HMGB1) (Gebremeskel and Johnston (2015) 6(39):41600-41619). In some embodiments, the chemotherapeutic agent induces immunogenic cell death (ICD). In some embodiments, the agent that induces ICD is an anthracycline. In some embodiments, the anthracycline is selected from doxorubicin, daunorubicin, epirubicin, idarubicin, and valrubicin. In some embodiments, the anthracycline is doxorubicin. In some embodiments, the agent that induces ICD is a platinum derivative. In some embodiments, the platinum derivative is selected from oxaliplatin, carboplatin, and cisplatin. In some embodiments, the platinum derivative is oxaliplatin.

### Hypomethylating Agents

In some aspects, the disclosure provides methods and compositions for treating or delaying progression of a cancer or reducing or inhibiting tumor growth in a subject in need thereof, by administering: an effective amount of an antibody that specifically binds human CD47, or antigen binding fragment thereof; an effective amount of a cell death-inducing agent; and an effective amount of a hypomethylating agent. In some aspects, the cell death-inducing agent is a targeted therapeutic agent selected from an agent that induces apoptosis, an agent that induces immunogenic cell death (ICD), and an agent that inhibits a DNA damage response pathway. In some aspects, the monoclonal antibody or antigen binding fragment is administered preceding or subsequent to administration of the cell death-inducing agent and preceding or subsequent to administration of the hypomethylating agent. In some aspects, the cell-death inducing agent is administered preceding or subsequent to administration of the monoclonal antibody of antigen binding fragment and preceding or subsequent to administration of the hypomethylating agent. In some aspects, the hypomethylating agent is administered preceding or subsequent to administration of the monoclonal antibody of antigen binding fragment and preceding or subsequent to administration of the cell death-inducing agent.

DNA methylation refers to the modification of DNA nucleotides by the addition of one or more methyl groups and is a common epigenetic modification that can result in altered gene expression. Increased methylation of tumor suppressor genes in some cancers contributes to the growth and survival of the cancer. However, the reversible nature of DNA methylation allows for the demethylation of the genes with DNA hypomethylating agents. Hypomethylating agents decrease the amount of cellular DNA methylation and subsequently reactivate the tumor suppressor genes. (Datta et al., Genes and Cancer, 3(1) 71-81, 2012).

The present disclosure provides methods and compositions for a combination therapy of an anti-CD47 antibody (or antigen binding fragment thereof), a cell death-inducing agent, and a hypomethylating agent. Hypomethylating agents inhibit DNA methylation by inhibiting the activity of the DNA methyltransferases. Exemplary hypomethylating agents of the disclosure are 5-azacitidine (5-AzaC or VIDAZA®) and 5-aza-2′-deoxycytidine (5-AzadC or decitabine or DACOGEN®). Both compounds are cytidine analogs, approved by the FDA, and are commercially available. 5-azacitidine is incorporated into both DNA and RNA. (Raj and Mufti Thera. and Clin. Risk Manag., 2006:2(4) 377-388). Once incorporated into DNA, it binds irreversibly to DNA methyltransferases, thereby blocking DNA methylation. 5-aza-2′-deoxycytidine is incorporated into DNA and acts through a similar mechanism. Both 5-azacitidine and 5-aza-2′-deoxycytidine have been used as single agents for the treatment of myelodysplastic syndromes (MDS), myeloproliferative neoplasms (MPN), and acute myeloid leukemia (AML).

Other hypomethylating agents useful in the methods and compositions of the disclosure include, for example, 5-fluoro-2′deoxycytidine, zebularine, antisense oligodeoxynucleotides, mitoxantrone, psammaplin A, procaine, N-acetylprocainamide, procainamide, hydralazine, and epigallocatechin-3-gallate. (Datta et al., Genes and Cancer, 3(1) 71-81, 2012).

### Methods of Making Antibodies

The disclosure also features methods for producing any of the antibodies or antigen-binding fragments thereof described herein. In some embodiments, methods for preparing an antibody described herein can include immunizing a subject (e.g., a non-human mammal) with an appropriate immunogen. Suitable immunogens for generating any of the antibodies described herein are set forth herein. For example, to generate an antibody that binds to CD47, a skilled artisan can immunize a suitable subject (e.g., a nonhuman mammal such as a rat, a mouse, a gerbil, a hamster, a dog, a cat, a pig, a goat, a horse, or a non-human primate) with a CD47 polypeptide, e.g., SEQ ID NO: 1.

A suitable subject (e.g., a non-human mammal) can be immunized with the appropriate antigen along with subsequent booster immunizations a number of times sufficient to elicit the production of an antibody by the mammal. The immunogen can be administered to a subject (e.g., a non-human mammal) with an adjuvant. Adjuvants useful in producing an antibody in a subject include, but are not limited to, protein adjuvants; bacterial adjuvants, e.g., whole bacteria (BCG, Corynebacterium parvum or Salmonella minnesota) and bacterial components including cell wall skeleton, trehalose dimycolate, monophosphoryl lipid A, methanol extractable residue (MER) of tubercle bacillus, complete or incomplete Freund's adjuvant; viral adjuvants; chemical adjuvants, e.g., aluminum hydroxide, and iodoacetate and cholesteryl hemisuccinate. Other adjuvants that can be used in the methods for inducing an immune response include, e.g., cholera toxin and parapoxvirus proteins. See also Bieg et al. (1999) Autoimmunity 31(1):15-24. See also, e.g., Lodmell et al. (2000) Vaccine 18:1059-1066; Johnson et al. (1999) J Med Chem 42:4640-4649; Baldridge et al. (1999) Methods 19:103-107; and Gupta et al. (1995) Vaccine 13(14): 1263-1276.

In some embodiments, the methods include preparing a hybridoma cell line that secretes a monoclonal antibody that binds to the immunogen. For example, a suitable mammal such as a laboratory mouse is immunized with a CD47 polypeptide as described above. Antibody-producing cells (e.g., B cells of the spleen) of the immunized mammal can be isolated two to four days after at least one booster immunization of the immunogen and then grown briefly in culture before fusion with cells of a suitable myeloma cell line. The cells can be fused in the presence of a fusion promoter such as, e.g., vaccinia virus or polyethylene glycol. The hybrid cells obtained in the fusion are cloned, and cell clones secreting the desired antibodies are selected. For example, spleen cells of Balb/c mice immunized with a suitable immunogen can be fused with cells of the myeloma cell line PAI or the myeloma cell line Sp2/0-Ag 14. After the fusion, the cells are expanded in suitable culture medium, which is supplemented with a selection medium, for example HAT medium, at regular intervals in order to prevent normal myeloma cells from overgrowing the desired hybridoma cells. The obtained hybrid cells are then screened for secretion of the desired antibodies (e.g., an antibody that binds to human CD47 blocks interaction with SIRPα).

In some embodiments, a skilled artisan can identify an antibody of interest from a non-immune biased library as described in, e.g., U.S. Pat. No. 6,300,064 (to Knappik et al.; Morphosys AG) and Schoonbroodt et al. (2005) Nucleic Acids Res 33(9):e81.

In some embodiments, the methods described herein can involve, or be used in conjunction with, e.g., phage display technologies, bacterial display, yeast surface display, eukaryotic viral display, mammalian cell display, and cell-free (e.g., ribosomal display) antibody screening techniques (see, e.g., Etz et al. (2001) J Bacteriol 183:6924-6935; Cornelis (2000) Curr Opin Biotechnol 11:450-454; Klemm et al. (2000) Microbiology 146:3025-3032; Kieke et al. (1997) Protein Eng 10:1303-1310; Yeung et al. (2002) Biotechnol Prog 18:212-220; Boder et al. (2000) Methods Enzymology 328:430-444; Grabherr et al. (2001) Comb Chem High Throughput Screen 4:185-192; Michael et al. (1995) Gene Ther 2:660-668; Pereboev et al. (2001) J Virol 75:7107-7113; Schaffitzel et al. (1999) J Immunol Methods 231:119-135; and Hanes et al. (2000) Nat Biotechnol 18:1287-1292).

Methods for identifying antibodies using various phage display methods are known in the art. In phage display methods, functional antibody domains are displayed on the surface of phage particles which carry the polynucleotide sequences encoding them. Such phage can be utilized to display antigen-binding domains of antibodies, such as Fab, Fv, or disulfide-bond stabilized Fv antibody fragments, expressed from a repertoire or combinatorial antibody library (e.g., human or murine). Phage used in these methods are typically filamentous phage such as fd and M13. The antigen binding domains are expressed as a recombinantly fused protein to any of the phage coat proteins pIII, pVIII, or pIX. See, e.g., Shi et al. (2010) JMB 397:385-396. Examples of phage display methods that can be used to make the immunoglobulins, or fragments thereof, described herein include those disclosed in Brinkman et al. (1995) J Immunol Methods 182:41-50; Ames et al. (1995) J Immunol Methods 184:177-186; Kettleborough et al. (1994) Eur J Immunol 24:952-958; Persic et al. (1997) Gene 187:9-18; Burton et al. (1994) Advances in Immunology 57:191-280; and PCT publication nos. WO 90/02809, WO 91/10737, WO 92/01047, WO 92/18619, WO 93/11236, WO 95/15982, and WO 95/20401. Suitable methods are also described in, e.g., U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108.

In some embodiments, the phage display antibody libraries can be generated using mRNA collected from B cells from the immunized mammals. For example, a splenic cell sample comprising B cells can be isolated from mice immunized with a CD47 polypeptide as described above. mRNA can be isolated from the cells and converted to cDNA using standard molecular biology techniques. See, e.g., Sambrook et al. (1989) “Molecular Cloning: A Laboratory Manual, 2nd Edition,” Cold Spring Harbor Laboratory Press, Cold Spring Harbor, N.Y.; Harlow and Lane (1988), supra; Benny K. C. Lo (2004), supra; and Borrebaek (1995), supra. The cDNA coding for the variable regions of the heavy chain and light chain polypeptides of immunoglobulins are used to construct the phage display library. Methods for generating such a library are described in, e.g., Merz et al. (1995) J Neurosci Methods 62(1-2):213-9; Di Niro et al. (2005) Biochem J 388(Pt 3):889-894; and Engberg et al. (1995) Methods Mol Biol 51:355-376.

In some embodiments, a combination of selection and screening can be employed to identify an antibody of interest from, e.g., a population of hybridoma-derived antibodies or a phage display antibody library. Suitable methods are known in the art and are described in, e.g., Hoogenboom (1997) Trends in Biotechnology 15:62-70; Brinkman et al. (1995), supra; Ames et al. (1995), supra; Kettleborough et al. (1994), supra; Persic et al. (1997), supra; and Burton et al. (1994), supra. For example, a plurality of phagemid vectors, each encoding a fusion protein of a bacteriophage coat protein (e.g., pIII, pVIII, or pIX of M13 phage) and a different antigen-combining region are produced using standard molecular biology techniques and then introduced into a population of bacteria (e.g., E. coli). Expression of the bacteriophage in bacteria can, in some embodiments, require use of a helper phage. In some embodiments, no helper phage is required (see, e.g., Chasteen et al., (2006) Nucleic Acids Res 34(21):e145). Phage produced from the bacteria are recovered and then contacted to, e.g., a target antigen bound to a solid support (immobilized). Phage may also be contacted to antigen in solution, and the complex is subsequently bound to a solid support.

A subpopulation of antibodies screened using the above methods can be characterized for their specificity and binding affinity for a particular antigen (e.g., human CD47) using any immunological or biochemical based method known in the art. For example, specific binding of an antibody to CD47 may be determined for example using immunological or biochemical based methods such as, but not limited to, an ELISA assay, SPR assays, immunoprecipitation assay, affinity chromatography, and equilibrium dialysis as described above. Immunoassays which can be used to analyze immunospecific binding and cross-reactivity of the antibodies include, but are not limited to, competitive and noncompetitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, fluorescent immunoassays, and protein A immunoassays. Such assays are routine and well known in the art.

In embodiments where the selected CDR amino acid sequences are short sequences (e.g., fewer than 10-15 amino acids in length), nucleic acids encoding the CDRs can be chemically synthesized as described in, e.g., Shiraishi et al. (2007) Nucleic Acids Symposium Series 51(1):129-130 and U.S. Pat. No. 6,995,259. For a given nucleic acid sequence encoding an acceptor antibody, the region of the nucleic acid sequence encoding the CDRs can be replaced with the chemically synthesized nucleic acids using standard molecular biology techniques. The 5′ and 3′ ends of the chemically synthesized nucleic acids can be synthesized to comprise sticky end restriction enzyme sites for use in cloning the nucleic acids into the nucleic acid encoding the variable region of the donor antibody.

In some embodiments, the anti-CD47 antibodies described herein comprise an altered heavy chain constant region that has reduced (or no) effector function relative to its corresponding unaltered constant region. Effector functions involving the constant region of the anti-CD47 antibody may be modulated by altering properties of the constant or Fc region. Altered effector functions include, for example, a modulation in one or more of the following activities: antibody-dependent cellular cytotoxicity (ADCC), complement-dependent cytotoxicity (CDC), apoptosis, binding to one or more Fc-receptors, and proinflammatory responses. Modulation refers to an increase, decrease, or elimination of an effector function activity exhibited by a subject antibody containing an altered constant region as compared to the activity of the unaltered form of the constant region. In particular embodiments, modulation includes situations in which an activity is abolished or completely absent.

An altered constant region with altered FcR binding affinity and/or ADCC activity and/or altered CDC activity is a polypeptide which has either an enhanced or diminished FcR binding activity and/or ADCC activity and/or CDC activity compared to the unaltered form of the constant region. An altered constant region which displays increased binding to an FcR binds at least one FcR with greater affinity than the unaltered polypeptide. An altered constant region which displays decreased binding to an FcR binds at least one FcR with lower affinity than the unaltered form of the constant region. Such variants which display decreased binding to an FcR may possess little or no appreciable binding to an FcR, e.g., 0 to 50% (e.g., less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%) of the binding to the FcR as compared to the level of binding of a native sequence immunoglobulin constant or Fc region to the FcR. Similarly, an altered constant region that displays modulated ADCC and/or CDC activity may exhibit either increased or reduced ADCC and/or CDC activity compared to the unaltered constant region. For example, in some embodiments, the anti-CD47 antibody comprising an altered constant region can exhibit approximately 0 to 50% (e.g., less than 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1%) of the ADCC and/or CDC activity of the unaltered form of the constant region. An anti-CD47 antibody described herein comprising an altered constant region displaying reduced ADCC and/or CDC may exhibit reduced or no ADCC and/or CDC activity.

In some embodiments, an anti-CD47 antibody described herein exhibits reduced or no effector function. In some embodiments, an anti-CD47 antibody described herein comprises a hybrid constant region, or a portion thereof, such as a G2/G4 hybrid constant region (see e.g., Burton et al. (1992) Adv Immun 51:1-18; Canfield et al. (1991) J Exp Med 173:1483-1491; and Mueller et al. (1997) Mol Immunol 34(6):441-452). See above.

In some embodiments, an anti-CD47 antibody may contain an altered constant region exhibiting enhanced or reduced complement dependent cytotoxicity (CDC). Modulated CDC activity may be achieved by introducing one or more amino acid substitutions, insertions, or deletions in an Fc region of the antibody. See, e.g., U.S. Pat. No. 6,194,551. Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See, e.g., Caron et al. (1992) J Exp Med 176:1191-1195 and Shopes (1992) Immunol 148:2918-2922; PCT publication nos. WO 99/51642 and WO 94/29351; Duncan and Winter (1988) Nature 322:738-40; and U.S. Pat. Nos. 5,648,260 and 5,624,821.

Any of the antibodies described herein can be screened and/or tested for their ability to modulate any of the activities or functions ascribed to either CD47, either in vitro or in vivo, using any immunological or biochemical-based methods known in the art.

### Recombinant Antibody Expression and Purification

The antibodies or antigen-binding fragments thereof described herein can be produced using a variety of techniques known in the art of molecular biology and protein chemistry. For example, a nucleic acid encoding one or both of the heavy and light chain polypeptides of an antibody can be inserted into an expression vector that contains transcriptional and translational regulatory sequences, which include, e.g., promoter sequences, ribosomal binding sites, transcriptional start and stop sequences, translational start and stop sequences, transcription terminator signals, polyadenylation signals, and enhancer or activator sequences. The regulatory sequences include a promoter and transcriptional start and stop sequences. In addition, the expression vector can include more than one replication system such that it can be maintained in two different organisms, for example in mammalian or insect cells for expression and in a prokaryotic host for cloning and amplification.

Several possible vector systems are available for the expression of cloned heavy chain and light chain polypeptides from nucleic acids in mammalian cells. One class of vectors relies upon the integration of the desired gene sequences into the host cell genome. Cells which have stably integrated DNA can be selected by simultaneously introducing drug resistance genes such as E. coli gpt (Mulligan and Berg (1981) Proc Natl Acad Sci USA 78:2072) or Tn5 neo (Southern and Berg (1982) Mol Appl Genet 1:327). The selectable marker gene can be either linked to the DNA gene sequences to be expressed, or introduced into the same cell by co-transfection (Wigler et al. (1979) Cell 16:77). A second class of vectors utilizes DNA elements which confer autonomously replicating capabilities to an extrachromosomal plasmid. These vectors can be derived from animal viruses, such as bovine papillomavirus (Sarver et al. (1982) Proc Natl Acad Sci USA, 79:7147), cytomegalovirus, polyoma virus (Deans et al. (1984) Proc Natl Acad Sci USA 81:1292), or SV40 virus (Lusky and Botchan (1981) Nature 293:79).

The expression vectors can be introduced into cells in a manner suitable for subsequent expression of the nucleic acid. The method of introduction is largely dictated by the targeted cell type, discussed below. Exemplary methods include CaPO4 precipitation, liposome fusion, cationic liposomes, electroporation, viral infection, dextran-mediated transfection, polybrene-mediated transfection, protoplast fusion, and direct microinjection.

Appropriate host cells for the expression of antibodies or antigen-binding fragments thereof include yeast, bacteria, insect, plant, and mammalian cells. Of particular interest are bacteria such as E. coli, fungi such as Saccharomyces cerevisiae and Pichia pastoris, insect cells such as SF9, mammalian cell lines (e.g., human cell lines), as well as primary cell lines.

In some embodiments, an antibody or fragment thereof can be expressed in, and purified from, transgenic animals (e.g., transgenic mammals). For example, an antibody can be produced in transgenic non-human mammals (e.g., rodents) and isolated from milk as described in, e.g., Houdebine (2002) Curr Opin Biotechnol 13(6):625-629; van Kuik-Romeijn et al. (2000) Transgenic Res 9(2): 155-159; and Pollock et al. (1999) J Immunol Methods 231(1-2): 147-157.

The antibodies and fragments thereof can be produced from the cells by culturing a host cell transformed with the expression vector containing nucleic acid encoding the antibodies or fragments, under conditions, and for an amount of time, sufficient to allow expression of the proteins. Such conditions for protein expression will vary with the choice of the expression vector and the host cell, and will be easily ascertained by one skilled in the art through routine experimentation. For example, antibodies expressed in E. coli can be refolded from inclusion bodies (see, e.g., Hou et al. (1998) Cytokine 10:319-30). Bacterial expression systems and methods for their use are well known in the art (see Current Protocols in Molecular Biology, Wiley & Sons, and Molecular Cloning—A Laboratory Manual—3^{rd} Ed., Cold Spring Harbor Laboratory Press, New York (2001)). The choice of codons, suitable expression vectors and suitable host cells will vary depending on a number of factors, and may be easily optimized as needed. An antibody (or fragment thereof) described herein can be expressed in mammalian cells or in other expression systems including but not limited to yeast, baculovirus, and in vitro expression systems (see, e.g., Kaszubska et al. (2000) Protein Expression and Purification 18:213-220).

Following expression, the antibodies and fragments thereof can be isolated. An antibody or fragment thereof can be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography. For example, an antibody can be purified using a standard anti-antibody column (e.g., a protein-A or protein-G column). Ultrafiltration and diafiltration techniques, in conjunction with protein concentration, are also useful. See, e.g., Scopes (1994) “Protein Purification, 3rd edition,” Springer-Verlag, New York City, N.Y. The degree of purification necessary will vary depending on the desired use. In some instances, no purification of the expressed antibody or fragments thereof will be necessary.

Methods for determining the yield or purity of a purified antibody or fragment thereof are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

### Modification of the Antibodies or Antigen-Binding Fragments Thereof

The antibodies or antigen-binding fragments thereof can be modified following their expression and purification. The modifications can be covalent or noncovalent modifications. Such modifications can be introduced into the antibodies or fragments by, e.g., reacting targeted amino acid residues of the polypeptide with an organic derivatizing agent that is capable of reacting with selected side chains or terminal residues. Suitable sites for modification can be chosen using any of a variety of criteria including, e.g., structural analysis or amino acid sequence analysis of the antibodies or fragments.

In some embodiments, the antibodies or antigen-binding fragments thereof can be conjugated to a heterologous moiety. The heterologous moiety can be, e.g., a heterologous polypeptide, a therapeutic agent (e.g., a toxin or a drug), or a detectable label such as, but not limited to, a radioactive label, an enzymatic label, a fluorescent label, a heavy metal label, a luminescent label, or an affinity tag such as biotin or streptavidin. Suitable heterologous polypeptides include, e.g., an antigenic tag (e.g., FLAG (DYKDDDDK (SEQ ID NO: 22)), polyhistidine (6-His; HHHHHH (SEQ ID NO: 23), hemagglutinin (HA; YPYDVPDYA (SEQ ID NO: 24)), glutathione-S-transferase (GST), or maltose-binding protein (MBP)) for use in purifying the antibodies or fragments. Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., 32P, 33P, 14C, 125I, 131I, 35S, and 3H. Suitable fluorescent labels include, without limitation, fluorescein, fluorescein isothiocyanate (FITC), green fluorescent protein (GFP), DyLight™ 488, phycoerythrin (PE), propidium iodide (PI), PerCP, PE-Alexa Fluor® 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Enzymatic labels include, e.g., alkaline phosphatase, CAT, luciferase, and horseradish peroxidase.

Two proteins (e.g., an antibody and a heterologous moiety) can be crosslinked using any of a number of known chemical cross linkers. Examples of such cross linkers are those which link two amino acid residues via a linkage that includes a “hindered” disulfide bond. In these linkages, a disulfide bond within the cross-linking unit is protected (by hindering groups on either side of the disulfide bond) from reduction by the action, for example, of reduced glutathione or the enzyme disulfide reductase. One suitable reagent, 4-succinimidyloxycarbonyl-α-methyl-α(2-pyridyldithio) toluene (SMPT), forms such a linkage between two proteins utilizing a terminal lysine on one of the proteins and a terminal cysteine on the other. Heterobifunctional reagents that cross-link by a different coupling moiety on each protein can also be used. Other useful cross-linkers include, without limitation, reagents which link two amino groups (e.g., N-5-azido-2-nitrobenzoyloxysuccinimide), two sulfhydryl groups (e.g., 1,4-bis-maleimidobutane), an amino group and a sulfhydryl group (e.g., mmaleimidobenzoyl-N-hydroxysuccinimide ester), an amino group and a carboxyl group (e.g., 4-[p-azidosalicylamido]butylamine), and an amino group and a guanidinium group that is present in the side chain of arginine (e.g., p-azidophenyl glyoxal monohydrate).

In some embodiments, a radioactive label can be directly conjugated to the amino acid backbone of the antibody. Alternatively, the radioactive label can be included as part of a larger molecule (e.g., 125I in meta-[125I]iodophenyl-N-hydroxysuccinimide ([125I]mIPNHS) which binds to free amino groups to form meta-iodophenyl (mIP) derivatives of relevant proteins (see, e.g., Rogers et al. (1997) J Nucl Med 38:1221-1229) or chelate (e.g., to DOTA or DTPA) which is in turn bound to the protein backbone. Methods of conjugating the radioactive labels or larger molecules/chelates containing them to the antibodies or antigen-binding fragments described herein are known in the art. Such methods involve incubating the proteins with the radioactive label under conditions (e.g., pH, salt concentration, and/or temperature) that facilitate binding of the radioactive label or chelate to the protein (see, e.g., U.S. Pat. No. 6,001,329).

Methods for conjugating a fluorescent label (sometimes referred to as a “fluorophore”) to a protein (e.g., an antibody) are known in the art of protein chemistry. For example, fluorophores can be conjugated to free amino groups (e.g., of lysines) or sulfhydryl groups (e.g., cysteines) of proteins using succinimidyl (NETS) ester or tetrafluorophenyl (TFP) ester moieties attached to the fluorophores. In some embodiments, the fluorophores can be conjugated to a heterobifunctional cross-linker moiety such as sulfo-SMCC. Suitable conjugation methods involve incubating an antibody protein, or fragment thereof, with the fluorophore under conditions that facilitate binding of the fluorophore to the protein. See, e.g., Welch and Redvanly (2003) “Handbook of Radiopharmaceuticals: Radiochemistry and Applications,” John Wiley and Sons (ISBN 0471495603).

In some embodiments, the antibodies or fragments can be modified, e.g., with a moiety that improves the stabilization and/or retention of the antibodies in circulation, e.g., in blood, serum, or other tissues. For example, the antibody or fragment can be PEGylated as described in, e.g., Lee et al. (1999) Bioconjug Chem 10(6): 973-8; Kinstler et al. (2002) Advanced Drug Deliveries Reviews 54:477-485; and Roberts et al. (2002) Advanced Drug Delivery Reviews 54:459-476 or HESylated (Fresenius Kabi, Germany; see, e.g., Pavisié et al. (2010) Int J Pharm 387(1-2):110-119). The stabilization moiety can improve the stability, or retention of, the antibody (or fragment) by at least about 1.5 (e.g., at about least 2, 5, 10, 15, 20, 25, 30, 40, or 50 or more) fold.

In some embodiments, the antibodies or antigen-binding fragments thereof described herein can be glycosylated. In some embodiments, an antibody or antigen-binding fragment thereof described herein can be subjected to enzymatic or chemical treatment, or produced from a cell, such that the antibody or fragment has reduced or absent glycosylation. Methods for producing antibodies with reduced glycosylation are known in the art and described in, e.g., U.S. Pat. No. 6,933,368; Wright et al. (1991) EMBO J 10(10):2717-2723; and Co et al. (1993) Mol Immunol 30:1361.

### Pharmaceutical Compositions and Formulations

In some embodiments, an anti-CD47 antibody, or antigen binding fragment thereof, and a cell death-inducing agent are administered together (simultaneously or sequentially). In some embodiments, an anti-CD47 antibody, or antigen binding fragment thereof, and a cell death-inducing agent are administered separately.

In certain embodiments, the disclosure provides for a pharmaceutical composition comprising an anti-CD47 antibody, or antigen-binding fragment thereof, with a pharmaceutically acceptable diluent, carrier, solubilizer, emulisifier, preservative and/or adjuvant, and a pharmaceutical composition comprising a cell death-inducing agent with a pharmaceutically acceptable diluent, carrier, solubilizer, emulsifier, preservative and/or adjuvant. In certain embodiments, each of the agents, e.g., anti-CD47 antibody, antigen-binding fragment thereof, cell death-inducing agent, can be formulated as separate composition.

In certain embodiments, acceptable formulation materials preferably are nontoxic to recipients at the dosages and concentrations employed. In certain embodiments, the formulation material(s) are for s.c. and/or I.V. administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995). In certain embodiments, the formulation comprises PBS; 20 mM NaOAC, pH 5.2, 50 mM NaCl; and/or 10 mM NAOAC, pH 5.2, 9% Sucrose. In certain embodiments, the optimal pharmaceutical composition will be determined by one skilled in the art depending upon, for example, the intended route of administration, delivery format and desired dosage. See, for example, Remington's Pharmaceutical Sciences, supra. In certain embodiments, such compositions may influence the physical state, stability, rate of in vivo release and/or rate of in vivo clearance of an anti-CD47 antibody and/or a cell death-inducing agent.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising an anti-CD47 antibody and a cell-death inducing agent can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising an anti-CD47 antibody and a cell death-inducing agent can be formulated as a lyophilizate using appropriate excipients such as sucrose.

In certain embodiments, the pharmaceutical composition can be selected for parenteral delivery. In certain embodiments, the compositions can be selected for inhalation or for delivery through the digestive tract, such as orally. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8.

In certain embodiments, when parenteral administration is contemplated, a therapeutic composition can be in the form of a pyrogen-free, parenterally acceptable aqueous solution comprising an anti-CD47 antibody and a cell death-inducing agent, in a pharmaceutically acceptable vehicle. In certain embodiments, a vehicle for parenteral injection is sterile distilled water in which an anti-CD47 antibody and a cell death-inducing agent is formulated as a sterile, isotonic solution, and properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, a pharmaceutical composition can be formulated for inhalation. In certain embodiments, an anti-CD47 antibody and a cell death-inducing agent can be formulated as a dry powder for inhalation. In certain embodiments, an inhalation solution comprising an anti-CD47 antibody and a cell death-inducing agent can be formulated with a propellant for aerosol delivery. In certain embodiments, solutions can be nebulized. Pulmonary administration is further described in PCT application No. PCT/US94/001875, which describes pulmonary delivery of chemically modified proteins.

In certain embodiments, it is contemplated that formulations can be administered orally. In certain embodiments, an anti-CD47 antibody and a cell death-inducing agent that is administered in this fashion can be formulated with or without those carriers customarily used in the compounding of solid dosage forms such as tablets and capsules. In certain embodiments, a capsule can be designed to release the active portion of the formulation at the point in the gastrointestinal tract when bioavailability is maximized and pre-systemic degradation is minimized. In certain embodiments, at least one additional agent can be included to facilitate absorption of an anti-CD47 antibody and a cell death-inducing agent. In certain embodiments, diluents, flavorings, low melting point waxes, vegetable oils, lubricants, suspending agents, tablet disintegrating agents, and binders can also be employed.

In certain embodiments, a pharmaceutical composition can involve an effective quantity of an anti-CD47 antibody and a cell death-inducing agent in a mixture with non-toxic excipients which are suitable for the manufacture of tablets. In certain embodiments, by dissolving the tablets in sterile water, or another appropriate vehicle, solutions can be prepared in unit-dose form. In certain embodiments, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving an anti-CD47 antibody and a cell death-inducing agent in sustained- or controlled delivery formulations. In certain embodiments, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In certain embodiments, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15: 167-277 (1981) and Langer, Chem. Tech., 12:98-105 (1982)), ethylene vinyl acetate (Langer et al., supra) or poly-D(−)-3-hydroxybutyric acid (EP 133,988). In certain embodiments, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In certain embodiments, this can be accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration. In certain embodiments, kits are provided for producing a single-dose administration unit. In certain embodiments, the kit can contain both a first container having a dried protein and a second container having an aqueous formulation. In certain embodiments, kits containing single and multi-chambered pre-filled syringes (e.g., liquid syringes and lyosyringes) are included.

In certain embodiments, the effective amount of a pharmaceutical composition comprising an anti-CD47 antibody and a cell death-inducing agent to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which an anti-CD47 antibody and a cell death-inducing agent is being used, the route of administration, and the size (body weight, body surface or organ size) and/or condition (the age and general health) of the patient. In certain embodiments, the clinician can titer the dosage and modify the route of administration to obtain the optimal therapeutic effect.

In certain embodiments, the frequency of dosing will take into account the pharmacokinetic parameters of an anti-CD47 antibody and a cell death-inducing agent in the formulation used. In certain embodiments, a clinician will administer the composition until a dosage is reached that achieves the desired effect. In certain embodiments, the composition can therefore be administered as a single dose or as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g. orally, through injection by intravenous, intraperitoneal, intracerebral (intra-parenchymal), intracerebroventricular, intramuscular, subcutaneously, intra-ocular, intraarterial, intraportal, or intralesional routes; by sustained release systems or by implantation devices. In certain embodiments, the compositions can be administered by bolus injection or continuously by infusion, or by implantation device. In certain embodiments, individual elements of the combination therapy may be administered by different routes. In certain embodiments, the composition can be administered locally via implantation of a membrane, sponge or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration. In certain embodiments, it can be desirable to use a pharmaceutical composition comprising an anti-CD277 antibody and a galectin-1 antagonist in an ex vivo manner. In such instances, cells, tissues and/or organs that have been removed from the patient are exposed to a pharmaceutical composition comprising an anti-CD47 antibody and a cell death-inducing agent after which the cells, tissues and/or organs are subsequently implanted back into the patient.

In certain embodiments, an anti-CD47 antibody and a cell death-inducing agent can be delivered by implanting certain cells that have been genetically engineered, using methods such as those described herein, to express and secrete the polypeptides. In certain embodiments, such cells can be animal or human cells, and can be autologous, heterologous, or xenogeneic. In certain embodiments, the cells can be immortalized. In certain embodiments, in order to decrease the chance of an immunological response, the cells can be encapsulated to avoid infiltration of surrounding tissues. In certain embodiments, the encapsulation materials are typically biocompatible, semi-permeable polymeric enclosures or membranes that allow the release of the protein product(s) but prevent the destruction of the cells by the patient's immune system or by other detrimental factors from the surrounding tissues.

### Kits

A kit can include an anti-CD47 antibody and a cell death-inducing agent as disclosed herein, and instructions for use. The kits may comprise, in a suitable container, an anti-CD47 antibody, a cell death-inducing agent, one or more controls, and various buffers, reagents, enzymes and other standard ingredients well known in the art. Certain embodiments include a kit with an anti-CD47 antibody with instructions for use in combination with a cell death-inducing agent to treat or delay progression of cancer in a subject. In some aspects the anti-CD47 antibody and a cell death-inducing agent are provided in separate vials.

In certain embodiments, a kit includes a composition comprising a monoclonal antibody that specifically binds human CD47, or an antigen binding fragment thereof, and an optional pharmaceutically acceptable carrier, for use in treating or delaying progression of cancer in a subject, wherein the treatment comprises administration of the monoclonal antibody, or antigen binding fragment thereof, in combination with a second composition, wherein the second composition comprises a cell-death inducing agent, and an optional pharmaceutically acceptable carrier.

In some aspects, the disclosure provides a kit comprising a container which includes at least one vial, well, test tube, flask, bottle, syringe, or other container means, into which an anti-CD47 antibody or a cell death-inducing agent may be placed, and in some instances, suitably aliquoted. Where an additional component is provided, the kit can contain additional containers into which this component may be placed. The kits can also include a means for containing an anti-CD47 antibody and a cell death-inducing agent and any other reagent containers in close confinement for commercial sale. Such containers may include injection or blowmolded plastic containers into which the desired vials are retained. Containers and/or kits can include labeling with instructions for use and/or warnings.

In some aspects, the disclosure provides a kit comprising a medicament comprising a composition comprising a monoclonal antibody that specifically binds human CD47, or an antigen binding fragment thereof, and an optional pharmaceutically acceptable carrier, and a package insert comprising instructions for administration of the medicament in combination with a second medicament comprising a composition comprising a cell-death inducing agent, and an optional pharmaceutically acceptable carrier, for treating or delaying progression of cancer in a subject.

In some aspects, the disclosure provides a kit comprising a container comprising a composition comprising a monoclonal antibody that specifically binds human CD47, or an antigen binding fragment thereof, and an optional pharmaceutically acceptable carrier, and a package insert comprising instructions for administration of the composition in combination with a second composition comprising a cell-death inducing agent, and an optional pharmaceutically acceptable carrier, for treating or delaying progression of cancer in a subject.

### Methods of Use

The compositions of the present invention have numerous in vitro and in vivo utilities. The above-described compositions are useful in, inter alia, methods for treating or preventing a variety of cancers in a subject. The compositions can be administered to a subject, e.g., a human subject, using a variety of methods that depend, in part, on the route of administration. The route can be, e.g., oral, sublingual, sublabial, buccal, rectal, vaginal, intraocular, intranasal, intraotic, inhalation, cutaneous, topical, systemic, transdermal, epidural, intracerebral, intracerebroventricular, intravenous injection or infusion (IV), subcutaneous injection (SC), intraperitoneal (IP) injection, intramuscular injection (IM), or intrathecal injection (IT). The injection can be in a bolus or a continuous infusion.

Administration can be achieved by, e.g., local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; EP488401; and EP 430539, the disclosures of each of which are incorporated herein by reference in their entirety. The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

In some embodiments, an anti-CD47 antibody, or antigen-binding fragment thereof, and a cell death-inducing agent are therapeutically delivered to a subject by way of local administration.

A suitable dose of an antibody, or antigen binding fragment thereof, and/or cell death-inducing agent described herein, which dose is capable of treating or preventing cancer in a subject, can depend on a variety of factors including, e.g., the age, sex, and weight of a subject to be treated and the particular inhibitor compound used. For example, a different dose of a whole anti-CD47 antibody may be required to treat a subject with cancer as compared to the dose of a CD47-binding Fab′ antibody fragment required to treat the same subject. Other factors affecting the dose administered to the subject include, e.g., the type or severity of the cancer. For example, a subject having metastatic melanoma may require administration of a different dosage of an anti-CD47 antibody and a cell death-inducing agent than a subject with glioblastoma. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject will also depend upon the judgment of the treating medical practitioner (e.g., doctor or nurse). Suitable dosages are described herein.

A pharmaceutical composition can include a therapeutically effective amount of an anti-CD47 antibody, or antigen binding fragment thereof, or a cell death-inducing agent described herein. Such effective amounts can be readily determined by one of ordinary skill in the art based, in part, on the effect of the administered antibody, or the combinatorial effect of the antibody and one or more additional active agents, if more than one agent is used. A therapeutically effective amount of an antibody or fragment thereof described herein can also vary according to factors such as the disease state, age, sex, and weight of the individual, and the ability of the antibody (and one or more additional active agents) to elicit a desired response in the individual, e.g., reduction in tumor growth. For example, a therapeutically effective amount of an anti-CD47 antibody and/or a cell death-inducing agent can inhibit (lessen the severity of or eliminate the occurrence of) and/or prevent a particular disorder, and/or any one of the symptoms of the particular disorder known in the art or described herein. A therapeutically effective amount is also one in which any toxic or detrimental effects of the composition are outweighed by the therapeutically beneficial effects.

Suitable human doses of any of the antibodies or antagonists described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1): 523-531; and Hetherington et al. (2006) Antimicrobial Agents and Chemotherapy 50(10): 3499-3500.

In some embodiments, the composition contains any of the antibodies or agents described herein and one or more (e.g., two, three, four, five, six, seven, eight, nine, 10, or 11 or more) additional therapeutic agents such that the composition as a whole is therapeutically effective. For example, a combination therapy may include administration of an anti-CD47 antibody and a cell death-inducing agent as described herein, and a chemotherapeutic agent, wherein the antibody, cell-death inducing agent and chemotherapeutic agent are each at a concentration that when combined are therapeutically effective for treating or preventing a cancer in a subject.

Toxicity and therapeutic efficacy of such compositions can be determined by known pharmaceutical procedures in cell cultures or experimental animals (e.g., animal models of any of the cancers described herein). These procedures can be used, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. An antibody or agent described herein that exhibits a high therapeutic index is preferred. While compositions that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. The dosage of such antibodies and agents described herein lies generally within a range of circulating concentrations of the antibodies or antagonists that include the ED50 with little or no toxicity. The dosage may vary within this range depending upon the dosage form employed and the route of administration utilized. For an anti-CD47 antibody and/or a cell death-inducing agent described herein, the therapeutically effective dose can be estimated initially from cell culture assays. A dose can be formulated in animal models to achieve a circulating plasma concentration range that includes the IC₅₀ (i.e., the concentration of the antibody which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration (e.g., to the eye or a joint) is desired, cell culture or animal modeling can be used to determine a dose required to achieve a therapeutically effective concentration within the local site.

In some embodiments, the methods can be performed in conjunction with other therapies for cancer. For example, the composition can be administered to a subject at the same time, prior to, or after, radiation, surgery, targeted or cytotoxic chemotherapy, chemoradiotherapy, hormone therapy, immunotherapy, gene therapy, cell transplant therapy, precision medicine, genome editing therapy, or other pharmacotherapy.

As described above, the compositions described herein (e.g., anti-CD47 antibody and cell death-inducing agent compositions) can be used to treat a variety of cancers such as but not limited to: Kaposi's sarcoma, leukemia, acute lymphocytic leukemia, acute myelocytic leukemia, myeloblasts promyelocyte myelomonocytic monocytic erythroleukemia, chronic leukemia, chronic myelocytic (granulocyti c) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, Kaposi's Sarcoma, skin cancer, stomach cancer, testicular cancer, thyroid cancer, uterine cancer, and cancer of the urinary system.

Combination Therapy

In some embodiments, an anti-CD47 antibody and a cell death-inducing agent described herein can be administered to a subject as a combination therapy, optionally with another treatment, e.g., another treatment for a cancer.

A combination of an anti-CD47 antibody and a cell death-inducing agent described herein can replace or augment a previously or currently administered therapy. For example, upon treating with a combination of an anti-CD47 antibody and a cell death-inducing agent, administration of one or more additional active agents can cease or diminish, e.g., be administered at lower levels. In some embodiments, administration of the previous therapy can be maintained. In some embodiments, a previous therapy will be maintained until the level of the anti-CD47 antibody and/or a cell death-inducing agent, reaches a level sufficient to provide a therapeutic effect. The two therapies can be administered in combination.

Monitoring a subject (e.g., a human patient) for an improvement in a cancer, as defined herein, means evaluating the subject for a change in a disease parameter, e.g., a reduction in tumor growth or prevention of tumor re-growth. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality, e.g., adding or dropping any of the treatments for a cancer described herein.

In some embodiments, the disclosure provides a method of treating cancer in a subject in need thereof, comprising administering to the subject a therapeutically-effective amount of a cell death-inducing agent and a therapeutically-effective amount of an antibody or antigen-binding fragment thereof that binds to human CD47, wherein the antibody or antigen-binding fragment thereof induces or enhances phagocytosis of CD47 expressing tumor cells, and wherein the cell death-inducing agent induces or enhances apoptosis of the tumor cells, to thereby treat the cancer.

In some embodiments, the disclosure provides a method of treating cancer in a subject in need thereof, comprising administering to the subject a therapeutically-effective amount of a cell death-inducing agent and a therapeutically-effective amount of an antibody or antigen-binding fragment thereof that binds to human CD47, wherein both the antibody or antigen-binding fragment thereof, and cell death-inducing agent, induce or enhance apoptosis of CD47 expressing tumor cells, to thereby treat the cancer.

In some embodiments, the disclosure provides a method of treating cancer in a subject in need thereof, comprising administering to the subject a therapeutically-effective amount of a cell death-inducing agent and a therapeutically-effective amount of an antibody or antigen-binding fragment thereof that binds to human CD47, wherein the cell death-inducing agent enhances expression or availability of CD47 on a tumor or tumor cells, such that cancer-specific targeting of the tumor or tumor cells by the antibody is enhanced, to thereby treat the cancer.

In some embodiments, the disclosure provides a method of treating cancer in a subject in need thereof, comprising administering to the subject a therapeutically-effective amount of a cell death-inducing agent and a therapeutically-effective amount of an antibody or antigen-binding fragment thereof that binds to human CD47, wherein the cell death-inducing agent causes release of soluble CD47, membrane-bound fragments or exosomes containing CD47 that are immunosuppressive, and wherein the antibody relieves the immunosuppressive effect to enhance an anti-tumor effect of the cell death-inducing agent, to thereby treat the cancer.

In some embodiments, the disclosure provides a method of treating cancer in a subject in need thereof, comprising administering to the subject a therapeutically-effective amount of a cell death-inducing agent and a therapeutically-effective amount of an antibody or antigen-binding fragment thereof that binds to human CD47, wherein the cell death-inducing agent stimulates early events of cell death, such as increased expression of phagocytosis-stimulating signals on the surface of tumor cells, and wherein the antibody blocks anti-phagocytic signals on the surface of tumor cells, such that phagocytosis of the tumor cells is enhanced, to thereby treat the cancer. In some embodiments, the cell death-inducing agent enhances expression of the phagocytic stimulating signal calreticulin.

In some embodiments, the cell death-inducing agent upregulates CD47 expression on the surface of tumor cells, thereby enhancing targeting of the antibody that specifically binds human CD47 toward the tumor.

In some embodiments, the disclosure provides methods of inducing death of tumor cells by contacting the tumor cells with an antibody that specifically binds human CD47 and a cell death-inducing agent. In some embodiments, the disclosure provides methods of inducing apoptosis of tumor cells by contacting the tumor cells with an antibody that specifically binds human CD47 and a cell death-inducing agent. In some embodiments, the disclosure provides methods of inducing phagocytosis of tumor cells by contacting the tumor cells with an antibody that specifically binds human CD47 and a cell death-inducing agent.

Accordingly, in one aspect, the disclosure provides methods and compositions for treating or delaying progression of a cancer or reducing or inhibiting tumor growth in a subject in need thereof, by administering: an effective amount of an antibody that specifically binds human CD47, or antigen binding fragment thereof; and an effective amount of a cell death-inducing agent. In some aspects, the combination therapy further includes an effective amount of a hypomethylating agent (e.g., 5-azacididine).

In some aspects, the cell death-inducing agent is selected from an agent that induces apoptosis, an agent that induces immunogenic cell death (ICD), and an agent that inhibits a DNA damage response pathway. In some aspects, the cancer is MDS, MPN or AML.

In other aspects, the disclosure provides methods and compositions for treating or delaying progression of a cancer or reducing or inhibiting tumor growth in a subject in need thereof, by administering: an effective amount of an antibody that specifically binds human CD47, or antigen binding fragment thereof; an effective amount of a cell death-inducing agent; and, optionally, an effective amount of a hypomethylating agent (e.g., 5-azacididine or 5-aza-2′-deoxycytidine). In some aspects, the cell death-inducing agent is selected from an agent that induces apoptosis, an agent that induces immunogenic cell death (ICD), and an agent that inhibits a DNA damage response pathway. In some aspects, the cancer is MDS, MPN or AML.

In some aspects, the disclosure provides methods and compositions for treating cancer (e.g., a hematological cancer) comprising administering a combination of an antibody that binds CD47, a cell death inducing agent (e.g., a BCL-2 inhibitor), and, optionally, a hypomethylating agent. In some embodiments, the disclosure provides methods and compositions for treating cancer comprising administering a combination of an antibody that binds CD47, a BCL-2 inhibitor, and, optionally, 5-azacitidine. In some embodiments, the BCL-2 inhibitor is ABT-199.

In some embodiments, the disclosure provides methods and compositions for treating cancer comprising administering a combination of an antibody that binds CD47, an anthracycline, and optionally, a hypomethylating agent. In some embodiments, the anthracycline is doxorubicin.

In some embodiments, the disclosure provides methods and compositions for treating cancer comprising administering a combination of an antibody that binds CD47, a proteasome inhibitor, and optionally, a hypomethylating agent. In some embodiments, the proteasome inhibitor is bortezomib.

In some embodiments, the disclosure provides methods and compositions for treating cancer comprising administering a combination of an antibody that binds CD47, a platinum derivative, and optionally, a hypomethylating agent. In some embodiments, the platinum derivative is oxaliplatin.

In some embodiments, the disclosure provides methods and compositions for treating cancer comprising administering a combination of an antibody that binds CD47, an inhibitor of PARP, and optionally, a hypomethylating agent. In some embodiments, the inhibitor of PARP is Olaparib.

In some embodiments, the disclosure provides methods and compositions for treating cancer comprising administering a combination of an antibody that binds CD47 and a cell death-inducing agent, wherein the antibody that binds CD47 is antibody 2.3D11 comprising:

a heavy chain complementarity determining region 1 (HC CDR1) comprising the amino acid sequence set forth in SEQ ID NO: 5;

a heavy chain complementarity determining region 2 (HC CDR2) comprising the amino acid sequence set forth in SEQ ID NO: 6;

a heavy chain complementarity determining region 3 (HC CDR3) comprising the amino acid sequence set forth in SEQ ID NO: 7;

a light chain complementarity determining region 1 (LC CDR1) comprising the amino acid sequence set forth in SEQ ID NO: 8;

a light chain complementarity determining region 2 (LC CDR2) comprising the amino acid sequence set forth in SEQ ID NO: 9; and

a light chain complementarity determining region 3 (LC CDR3) comprising the amino acid sequence set forth in SEQ ID NO: 10.

In some embodiments, the disclosure provides methods and compositions for treating cancer comprising administering a combination of an antibody that binds CD47 and a cell death-inducing agent, wherein the antibody that binds CD47, or antigen binding fragment thereof, comprises a heavy chain variable region (VH) comprising the amino acid sequence set forth in SEQ ID NO: 3 and a light chain variable region (V_{L}) comprising the amino acid sequence set forth in SEQ ID NO: 4. In some embodiments, the anti-CD47 antibody comprises a VH region as set forth in SEQ ID NO: 3, a V_{L} region as set forth in SEQ ID NO: 4 and a wild-type human IgG4. In some embodiments, the anti-CD47 antibody comprises a VH region as set forth in SEQ ID NO: 3, a V_{L} region as set forth in SEQ ID NO: 4 and a wild-type human IgG1. In some embodiments, the anti-CD47 antibody comprises a mutant human IgG1 or a mutant human IgG4 heavy chain constant region. In some embodiments, the mutant human IgG1 heavy chain constant region comprises a substitution at Glu233, Leu234, Leu235, Asn297, or a combination thereof, numbering according to EU numbering. In some embodiments, the mutant IgG1 heavy chain constant region comprises an E233P substitution, an L234A or L234E substitution, an L235A substitution, an N297A substitution, or a combination thereof, numbering according to EU numbering. In some embodiments, the mutant IgG4 heavy chain constant region comprises a substitution at Ser228, Leu235, Asn297, or a combination thereof, numbering according to EU numbering. In some embodiments, the mutant IgG4 heavy chain constant region comprises an S228P substitution, an L235E substitution, an N297A substitution, or a combination thereof, numbering according to EU numbering. In some embodiments, the mutant IgG4 heavy chain constant region comprises an S228P substitution and an L235E substitution, numbering according to EU numbering.

In some embodiments, the anti-CD47 antibody comprises a VH region as set forth in SEQ ID NO: 3, a V_{L} region as set forth in SEQ ID NO: 4, a wild-type human IgG4 and a human kappa constant region. In some aspects, the wild-type human IgG4 heavy chain constant region comprises the amino acid sequence set forth in SEQ ID NO: 21. In some aspects, the human kappa constant region comprises the amino acid sequence set forth in SEQ ID NO: 17.

In some embodiments, the disclosure provides methods and compositions for treating cancer comprising administering a combination of an antibody that binds CD47 and a cell death-inducing agent, wherein the antibody that binds CD47, wherein the antibody comprises a heavy chain comprising the amino acid sequence set forth in SEQ ID NO: 11, SEQ ID NO: 12, SEQ ID NO: 13, or SEQ ID NO: 14; and a light chain comprising the amino acid sequence set forth in SEQ ID NO: 15 or SEQ ID NO: 16. In some aspects, the anti-CD47 antibody comprises a heavy chain comprising the amino acid sequence set forth in SEQ ID NO: 13; and a light chain comprising the amino acid sequence set forth in SEQ ID NO: 16.

In some embodiments, the disclosure provides methods and compositions for treating cancer comprising administering a combination of an antibody that binds CD47, a BCL-2 inhibitor, and, optionally, 5-azacitidine, wherein the antibody comprises a heavy chain comprising the amino acid sequence set forth in SEQ ID NO: 11, SEQ ID NO: 12, SEQ ID NO: 13, or SEQ ID NO: 14; and a light chain comprising the amino acid sequence set forth in SEQ ID NO: 15 or SEQ ID NO: 16. In some aspects, the anti-CD47 antibody comprises a heavy chain comprising the amino acid sequence set forth in SEQ ID NO: 13; and a light chain comprising the amino acid sequence set forth in SEQ ID NO: 16.

In some embodiments, the disclosure provides methods and compositions for treating cancer comprising administering a combination of an antibody that binds CD47, a BCL-2 inhibitor, and, optionally decitabine, wherein the antibody comprises a heavy chain comprising the amino acid sequence set forth in SEQ ID NO: 11, SEQ ID NO: 12, SEQ ID NO: 13, or SEQ ID NO: 14; and a light chain comprising the amino acid sequence set forth in SEQ ID NO: 15 or SEQ ID NO: 16. In some aspects, the anti-CD47 antibody comprises a heavy chain comprising the amino acid sequence set forth in SEQ ID NO: 13; and a light chain comprising the amino acid sequence set forth in SEQ ID NO: 16.

In some aspects, the disclosure provides methods and compositions for treating cancer comprising administering a combination of an antibody that binds CD47, a cell death-inducing agent, and, optionally a hypomethylating agent (e.g., 5-azacitidne), wherein the antibody comprises a heavy chain comprising the amino acid sequence set forth in SEQ ID NO: 13; and a light chain comprising the amino acid sequence set forth in SEQ ID NO: 16 and wherein the cell death-inducing agent is selected from an agent that induces apoptosis, an agent that induces immunogenic cell death (ICD), and an agent that inhibits a DNA damage response pathway.

In some aspects, the disclosure provides methods and compositions for treating cancer comprising administering a combination of an antibody that binds CD47, a BCL-2 (e.g., ABT-199) and, optionally a hypomethylating agent, wherein the antibody comprises a heavy chain comprising the amino acid sequence set forth in SEQ ID NO: 13; and a light chain comprising the amino acid sequence set forth in SEQ ID NO: 16.

In some aspects, the disclosure provides methods and compositions for treating cancer comprising administering a combination of an antibody that binds CD47, an anthracycline (e.g., doxorubicin) and, optionally a hypomethylating agent, wherein the antibody comprises a heavy chain comprising the amino acid sequence set forth in SEQ ID NO: 13; and a light chain comprising the amino acid sequence set forth in SEQ ID NO: 16.

In some aspects, the disclosure provides methods and compositions for treating cancer comprising administering a combination of an antibody that binds CD47, a proteasome inhibitor (e.g., bortezomib) and, optionally a hypomethylating agent, wherein the antibody comprises a heavy chain comprising the amino acid sequence set forth in SEQ ID NO: 13; and a light chain comprising the amino acid sequence set forth in SEQ ID NO: 16.

In some aspects, the disclosure provides methods and compositions for treating cancer comprising administering a combination of an antibody that binds CD47, a platinum derivative (e.g., oxaliplatin) and, optionally a hypomethylating agent, wherein the antibody comprises a heavy chain comprising the amino acid sequence set forth in SEQ ID NO: 13; and a light chain comprising the amino acid sequence set forth in SEQ ID NO: 16.

In some aspects, the disclosure provides methods and compositions for treating cancer comprising administering a combination of an antibody that binds CD47, an inhibitor of PARP (e.g., Olaparib) and, optionally a hypomethylating agent, wherein the antibody comprises a heavy chain comprising the amino acid sequence set forth in SEQ ID NO: 13; and a light chain comprising the amino acid sequence set forth in SEQ ID NO: 16.

In some embodiments, the cancer is a hematological cancer (e.g., acute myeloid leukemia, multiple myeloma). In some embodiments, the cancer is acute myeloid leukemia (AML).

In some embodiments, a combination of an antibody that binds CD47 and a cell death inducing agent (e.g., a BCL-2 inhibitor) enhances the treatment of cancer in the subject. In some aspects, the treatment of cancer is enhanced by at least 1%, at least 5%, at least 10%, at least 20%, at least 30%, at least 40%, at least 50%, at least 60%, at least 70%, at least 80%, at least 90%, at least 100%, or more relative to the administration of each agent individually.

In some embodiments, the combination of an antibody that binds CD47 and a cell death inducing agent (e.g., a BCL-2 inhibitor) reduces or inhibits tumor growth in the subject. In some aspects, tumor growth is reduced or inhibited by at least 1%, at least 5%, at least 10%, at least 20%, at least 30%, at least 40%, at least 50%, at least 60%, at least 70%, at least 80%, at least 90%, at least 100%, or more relative to the administration of each agent individually.

In some embodiments, the combination of an antibody that binds CD47 and a cell death inducing agent (e.g., a BCL-2 inhibitor) enhances the induction of apoptosis or induction of ICD of tumor cells the subject. In some aspects, apoptosis or ICD is enhanced by at least 1%, at least 5%, at least 10%, at least 20%, at least 30%, at least 40%, at least 50%, at least 60%, at least 70%, at least 80%, at least 90%, at least 100%, or more relative to the administration of each agent individually.

In some embodiments, the combination of an antibody that binds CD47 and a cell death inducing agent (e.g., a BCL-2 inhibitor) enhances the survival of a subject with cancer. In some aspects, survival is enhanced by at least 1%, at least 5%, at least 10%, at least 20%, at least 30%, at least 40%, at least 50%, at least 60%, at least 70%, at least 80%, at least 90%, at least 100%, or more relative to the administration of each agent individually.

In some aspects, the disclosure provides methods and compositions for treating cancer (e.g., a hematological cancer) comprising administering a combination of an antibody that binds CD47, a cell death inducing agent (e.g., a BCL-2 inhibitor), and, optionally, a hypomethylating agent. In some embodiments, the disclosure provides methods and compositions for treating cancer comprising administering a combination of an antibody that binds CD47, a BCL-2 inhibitor, and 5-azacitidine. In some embodiments, the disclosure provides methods and compositions for treating cancer comprising administering a combination of an antibody that binds CD47, a BCL-2 inhibitor, and decitabine. In some embodiments, the BCL-2 inhibitor is ABT-199.

In other aspects, the disclosure provides methods and compositions for treating or delaying progression of a cancer or reducing or inhibiting tumor growth in a subject in need thereof, by administering: an effective amount of an antibody that specifically binds human CD47, or antigen binding fragment thereof; an effective amount of ABT-199; and an effective amount of 5-azacididine or 5-aza-2′-deoxycytidine. The combined use of ABT-199 in combination with azacitidine or decitabine is being investigated in a phase 1b clinical trial (NCT02203773). A phase 3 clinical trial for the combined use of ABT-199 and azacitidine versus azacitidine in subjects with AML is recruiting patients (NCT02993523). In addition, a phase 1b clinical trial for the combined use of an antibody designed to block CD47 and azacitidine is recruiting patients (NCT03248479). Accordingly, some aspects, the disclosure provides methods and compositions for treating or delaying progression of AML in a subject in need thereof, by administering: an effective amount of an antibody that specifically binds human CD47, or antigen binding fragment thereof; an effective amount of a ABT-199; and an effective amount of azacitidine.

In some embodiments, the triple combination of an antibody that binds CD47, a cell death inducing agent (e.g., a BCL-2 inhibitor) and a hypomethylating agent enhances the treatment of cancer in the subject. In some aspects, the treatment of cancer is enhanced by at least 1%, at least 5%, at least 10%, at least 20%, at least 30%, at least 40%, at least 50%, at least 60%, at least 70%, at least 80%, at least 90%, at least 100%, or more relative to the administration of each agent individually.

In some embodiments, the triple combination of an antibody that binds CD47, a cell death inducing agent (e.g., a BCL-2 inhibitor) and a hypomethylating agent reduces or inhibits tumor growth in the subject. In some aspects, tumor growth is reduced or inhibited by at least 1%, at least 5%, at least 10%, at least 20%, at least 30%, at least 40%, at least 50%, at least 60%, at least 70%, at least 80%, at least 90%, at least 100%, or more relative to the administration of each agent individually.

In some embodiments, the triple combination of an antibody that binds CD47, a cell death inducing agent (e.g., a BCL-2 inhibitor) and a hypomethylating agent enhances the induction of apoptosis or ICD of tumor cells the subject. In some aspects, apoptosis or ICD is enhanced by at least 1%, at least 5%, at least 10%, at least 20%, at least 30%, at least 40%, at least 50%, at least 60%, at least 70%, at least 80%, at least 90%, at least 100%, or more relative to the administration of each agent individually.

In some embodiments, the triple combination of an antibody that binds CD47, a cell death inducing agent (e.g., a BCL-2 inhibitor) and a hypomethylating agent enhances the survival of a subject with cancer. In some aspects, survival is enhanced by at least 1%, at least 5%, at least 10%, at least 20%, at least 30%, at least 40%, at least 50%, at least 60%, at least 70%, at least 80%, at least 90%, at least 100%, or more relative to the administration of each agent individually.

In some embodiments, the combination therapy can include administering to the subject (e.g., a human patient) one or more additional agents that provide a therapeutic benefit to a subject who has, or is at risk of developing, cancer. Chemotherapeutic agents suitable for co-administration with compositions of the present invention include, for example: taxol, cytochalasin B, gramicidin D, ethidium bromide, emetine, mitomycin, etoposide, tenoposide, vincristine, vinblastine, colchicin, doxorubicin, daunorubicin, dihydroxyanthrancindione, mitoxantrone, mithramycin, actinomycin D, 1-dehydrotestosterone, glucocorticoids, procaine, tetracaine, lidocaine, propranolol, and puromycin and analogs or homologs thereof. Further agents include, for example, antimetabolites (e.g., methotrexate, 6-mercaptopurine, 6-thioguanine, cytarabine, 5-fluorouracil decarbazine), alkylating agents (e.g. mechlorethamine, thioTEPA, chlorambucil, melphalan, carmustine (BSNU), lomustine (CCNU), cyclophosphamide, busulfan, dibromomannitol, streptozotocin, mitomycin C, cis-dichlordiamine platinum (II)(DDP), procarbazine, altretamine, cisplatin, carboplatin, oxaliplatin, nedaplatin, satraplatin, or triplatin tetranitrate), anthracycline (e.g. daunorubicin (formerly daunomycin) and doxorubicin), antibiotics (e.g. dactinomcin (formerly actinomycin), bleomycin, mithramycin, and anthramycin (AMC)), and anti-mitotic agents (e.g. vincristine and vinblastine) and temozolomide.

In some embodiments, an anti-CD47 antibody and/or a cell death-inducing agent and the one or more additional active agents are administered at the same time. In other embodiments, the anti-CD47 antibody and/or cell death-inducing agent is administered first in time and the one or more additional active agents are administered second in time. In some embodiments, the one or more additional active agents are administered first in time and the anti-CD47 antibody and/or cell death-inducing agent is administered second in time.

### OTHER EMBODIMENTS

In one aspect, the present disclosure relates to a method for treating or delaying progression of a cancer in a subject in need thereof, the method comprising administering to the subject an effective amount of a monoclonal antibody that specifically binds human CD47, or antigen binding fragment thereof, and an effective amount of a cell death-inducing agent.

In another aspect, the present disclosure related to a method of reducing or inhibiting tumor growth in a subject in need thereof, the method comprising administering to the subject an effective amount of a monoclonal antibody that specifically binds human CD47, or antigen binding fragment thereof, and an effective amount of a cell death-inducing agent.

In yet another aspect, the present disclosure relates to a method of inducing apoptosis of tumor cells in a subject in need thereof, the method comprising administering to the subject an effective amount of monoclonal antibody that specifically binds human CD47, or antigen binding fragment thereof, and an effective amount of a cell death-inducing agent.

In another aspect, the present disclosure relates to a composition comprising a monoclonal antibody that specifically binds human CD47, or an antigen binding fragment thereof, and an optional pharmaceutically acceptable carrier, for use in treating or delaying progression of cancer in a subject, wherein the treatment comprises administration of the monoclonal antibody, or antigen binding fragment thereof, in combination with a second composition, wherein the second composition comprises a cell-death inducing agent, and an optional pharmaceutically acceptable carrier.

In one embodiment, the cell death-inducing agent is a targeted therapeutic agent. In some embodiments, the targeted therapeutic agent is selected from an agent that induces apoptosis, an agent that inhibits a DNA damage response pathway, a kinase inhibitor, or a proteasome inhibitor.

In some embodiments, the targeted therapeutic agent is an agent that induces apoptosis selected from the group consisting of: an inhibitor of BCL-2, an inhibitor of MCL-1, an inhibitor of Bcl-XL, and an inhibitor of MDM2. In one embodiment, the inhibitor of BCL-2 is Venetoclax™, Navitoclax, or obatoclax. In one embodiment, the MCL-1 inhibitor is AMG-176, the Bcl-XL inhibitor is WEHI-539 and the MDM2 inhibitor is AMG232.

In some embodiments, the targeted therapeutic agent is an agent that inhibits a DNA damage response pathway selected from an inhibitor of poly ADP ribose polymerase (PARP). In one embodiment, the inhibitor of PARP is Olapirib, Niraparib or Rucaparib.

In some embodiments, the targeted therapeutic agent is an agent that inhibits a DNA damage response pathway selected from temozolomide.

In some embodiments, the targeted therapeutic agent is a kinase inhibitor, wherein the kinase inhibitor is BTK inhibitor ibrutinib or is an inhibitor of phosphoinositide 3-kinase (PI3Kd) or spleen tyrosine kinase (Syk).

In some embodiments, the targeted therapeutic agent is a proteasome inhibitor. In one embodiment, the proteasome inhibitor is bortezomib or ixazomib.

In some embodiments, the cell-death inducing agent is a cytotoxic chemotherapeutic agent. In one embodiment, the cytotoxic chemotherapeutic agent is selected from the group consisting of: anthracyclines, topoisomerase inhibitors, bleomycin, gemcitabine, platins, taxanes, DNA alkylating agents, CHOP and fluorouracil.

In one embodiment of the present disclosure, the cell death-inducing agent enhances surface expression of CD47 on tumor cells. In some embodiments of the present disclosure, the cell death-inducing agent enhances release of soluble CD47. In some embodiments, the cell death-inducing agent enhances release of membrane-bound fragments or exosomes comprising CD47. In some embodiments, the cell death inducing agent enhances expression of at least one signal that induces phagocytosis. In one embodiment, the at least one signal that induces phagocytosis is calreticulin.

In one embodiment of the present disclosure, the monoclonal antibody that specifically binds human CD47, or antigen binding fragment thereof, binds to human CD47 expressed on tumor cells. In some embodiments, the monoclonal antibody that specifically binds human CD47, or antigen binding fragment thereof, blocks the interaction between CD47 and SIRPα. In some embodiments, blocking the interaction between CD47 and SIRPα induces macrophage phagocytosis of tumor cells expressing CD47.

In one embodiment of the present disclosure, the monoclonal antibody that specifically binds human CD47, or antigen binding fragment thereof, does not cause significant hemagglutination of human erythrocytes.

In some embodiments of the present disclosure, the monoclonal antibody that specifically binds human CD47, or antigen binding fragment thereof, includes a heavy chain complementarity determining region 1 (HC CDR1) comprising the amino acid sequence set forth in SEQ ID NO: 5; a heavy chain complementarity determining region 2 (HC CDR2) comprising the amino acid sequence set forth in SEQ ID NO: 6; a heavy chain complementarity determining region 3 (HC CDR3) comprising the amino acid sequence set forth in SEQ ID NO: 7; a light chain complementarity determining region 1 (LC CDR1) comprising the amino acid sequence set forth in SEQ ID NO: 8; a light chain complementarity determining region 2 (LC CDR2) comprising the amino acid sequence set forth in SEQ ID NO: 9; and a light chain complementarity determining region 3 (LC CDR3) comprising the amino acid sequence set forth in SEQ ID NO: 10.

In one embodiment, the monoclonal antibody that specifically binds to human CD47, or antigen binding fragment thereof, comprises a heavy chain variable region (VH) comprising the amino acid sequence set forth in SEQ ID NO: 3 and a light chain variable region (V_{L}) comprising the amino acid sequence set forth in SEQ ID NO: 4.

In some embodiments of the present disclosure, the monoclonal antibody that specifically binds human CD47, or antigen binding fragment thereof, is a human antibody. In some embodiments, the monoclonal antibody that specifically binds human CD47, or antigen binding fragment thereof, comprises a wild-type human IgG1 or a wild-type human IgG4 heavy chain constant region. In one embodiment, the wild-type human IgG4 heavy chain constant region comprises the amino acid sequence set forth in SEQ ID NO: 21.

In some embodiments, the monoclonal antibody that specifically binds human CD47, or antigen binding fragment thereof, comprises a mutant human IgG1 or a mutant human IgG4 heavy chain constant region. In one embodiment, the mutant human IgG1 heavy chain constant region comprises a substitution at Glu233, Leu234, Leu235, Asn297, or a combination thereof, numbering according to EU numbering. In one embodiment, the mutant IgG1 heavy chain constant region comprises an E233P substitution, an L234A or L234E substitution, an L235A substitution, an N297A substitution, or a combination thereof, numbering according to EU numbering. In one embodiment, the mutant IgG4 heavy chain constant region comprises a substitution at Ser228, Leu235, Asn297, or a combination thereof, numbering according to EU numbering. In one embodiment, the mutant IgG4 heavy chain constant region comprises an S228P substitution, an L235E substitution, an N297A substitution, or a combination thereof, numbering according to EU numbering. In one embodiment, the mutant IgG4 heavy chain constant region comprises an S228P substitution and an L235E substitution, numbering according to EU numbering.

In some embodiments of the present disclosure, the monoclonal antibody that specifically binds human CD47, or antigen binding fragment thereof, comprises a human kappa constant region. In one embodiment, the human kappa constant region comprises the amino acid sequence set forth in SEQ ID NO: 17.

In some embodiments of the present disclosure, the monoclonal antibody that specifically binds human CD47, or antigen binding fragment thereof, comprises a heavy chain comprising the amino acid sequence set forth in SEQ ID NO: 11, SEQ ID NO: 12, SEQ ID NO: 13, or SEQ ID NO: 14; and a light chain comprising the amino acid sequence set forth in SEQ ID NO: 15 or SEQ ID NO: 16.

In one embodiment of the present disclosure, the monoclonal antibody that specifically binds human CD47, or antigen binding fragment thereof, comprises a heavy chain comprising the amino acid sequence set forth in SEQ ID NO: 13; and a light chain comprising the amino acid sequence set forth in SEQ ID NO: 16.

In some embodiments of the present disclosure, the monoclonal antibody or antigen binding fragment is administered preceding or subsequent to administration of the cell-death inducing agent or wherein the cell-death inducing agent is administered preceding or subsequent to administration of the monoclonal antibody of antigen binding fragment.

In some embodiments of the present disclosure, the cancer or tumor is a hematological cancer or hematological tumor. In some embodiments, the hematological cancer or tumor is chronic lymphocytic leukemia (CLL), acute lymphocytic leukemia (ALL), chronic myelogenous leukemia (CML), diffuse large cell B cell lymphoma (DLBCL), follicular lymphoma (FL), Non-Hodgkin's lymphoma, myelofibrosis, mastocytosis, mantle cell lymphoma, multiple myeloma (MM) or acute myeloid leukemia (AML).

In some embodiments of the present disclosure, the cancer or tumor is a cancer or tumor of a tissue selected from the group consisting of lung, pancreas, breast, liver, ovary, testicle, kidney, bladder, spine, brain, cervix, endrometrium, colon/rectum, anus, esophagus, gallbladder, gastrointestinal tract, skin, prostate, testicle, pituitary, stomach, uterus, vagina and thyroid.

Other aspects of the disclosure relate to the use of a composition comprising a monoclonal antibody that specifically binds human CD47, or antigen-binding fragment thereof, as disclosed herein, and an optional pharmaceutically acceptable carrier, in the manufacture of a medicament for treating or delaying progression of cancer in a subject, wherein the medicament comprises the composition and an optional pharmaceutically acceptable carrier, and wherein the treatment comprises administration of the medicament in combination with a second composition, wherein the second composition comprises a cell-death inducing agent, and an optional pharmaceutically acceptable carrier.

In other aspects, the present disclosure relates to a kit comprising a medicament comprising a composition comprising a monoclonal antibody that specifically binds human CD47, or antigen-binding fragment thereof, as disclosed herein, and an optional pharmaceutically acceptable carrier, and a package insert comprising instructions for administration of the medicament in combination with a second medicament comprising a composition comprising a cell-death inducing agent, and an optional pharmaceutically acceptable carrier, for treating or delaying progression of cancer in a subject.

In some aspects, the kit comprises a container comprising a composition comprising a monoclonal antibody that specifically binds human CD47, or antigen-binding fragment thereof, as disclosed herein, and an optional pharmaceutically acceptable carrier, and a package insert comprising instructions for administration of the composition in combination with a second composition comprising a cell-death inducing agent, and an optional pharmaceutically acceptable carrier, for treating or delaying progression of cancer in a subject.

Aspects of the invention will be illustrated in view of the following figures and examples.

### Examples

### Example 1: Induction of Cell Death Via Anti-CD47 Antibody

An anti-CD47 antibody, 2.3D11, was previously found to potently inhibit the interaction between CD47 and SIRPα, enhance phagocytosis of tumor cells, cross compete with reference antibody B6H12 for binding to CD47, and not induce hemagglutination or red blood cell phagocytosis (US 2017/0081407, herein incorporated by reference in its entirety). In this example, antibody 2.3D11, comprising a heavy chain and light chain as set forth in SEQ ID NOs: 13 and 16, was further analyzed to determine whether cell death of target cells only occurred via phagocytosis. Jurkat cells were utilized as the target cells due to their expression of CD47 on their surface.

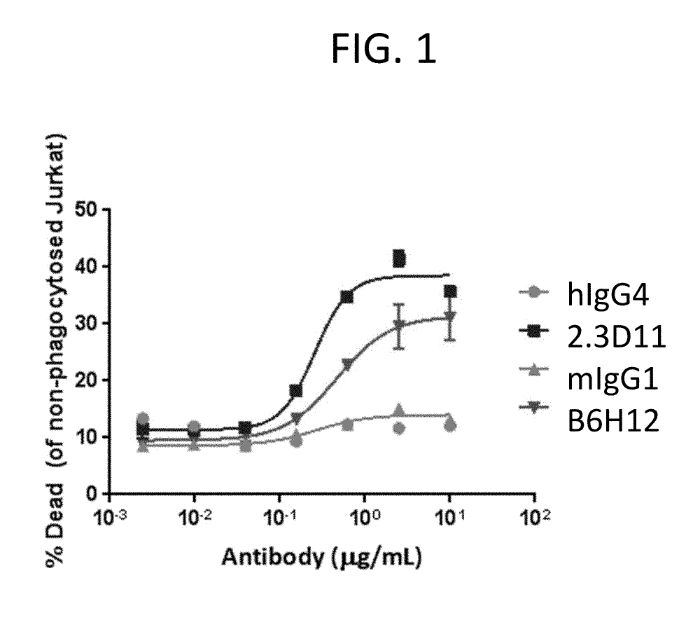

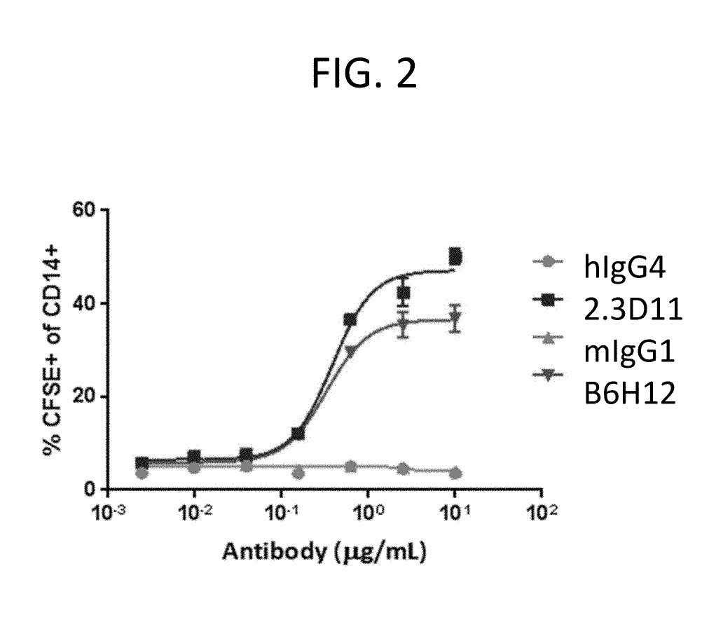

Briefly, effector cells (primary human macrophages (CD14+monocytes isolated from human peripheral blood and differentiated with M-CSF for 7 days)), were co-cultured with carboxyfluorescien succinimidyl ester (CFSE)-labeled Jurkat cells at a ratio of 1:2, for 2 hours in the presence of anti-CD47 antibodies 2.3D11 or B6H12, or isotype controls (i.e., human IgG4 and mouse IgG1). Non-phagocytosed Jurkat cells (CFSE+CD14-) were isolated by flow-cytometry and analyzed for Live/Dead staining using a Live/Dead Violet kit (Life Technologies). FIG. 1 shows the percentage of dead non-phagocytosed Jurkat cells. FIG. 2 shows the percentage of phagocytosed cells (CFSE+CD14+) induced by the anti-CD47 antibodies. These results indicated antibody 2.3D11 induces death of CD47 expressing cells via phagocytosis and non-phagocytosis mechanisms. Without being bound by theory, it is believed that 2.3D11-induced triggering of cell death pathways in target cells may enhance target cell uptake by macrophages.

### Example 2: Anti-CD47 Antibody Induced Cell Death is Caspase-Independent and PLCγ1 Dependent

Jurkat cells were utilized to determine whether the induction of cell death by an anti-CD47 antibody was caspase and/or PLCγ1 dependent. The anti-CD47 antibody was antibody 2.3D11 as described in Example 1.

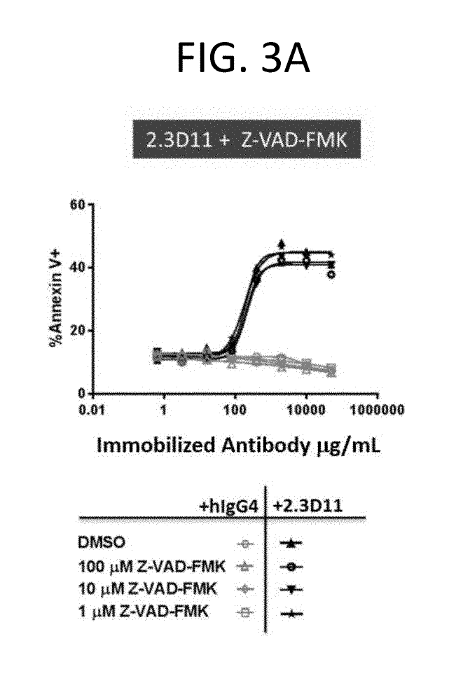

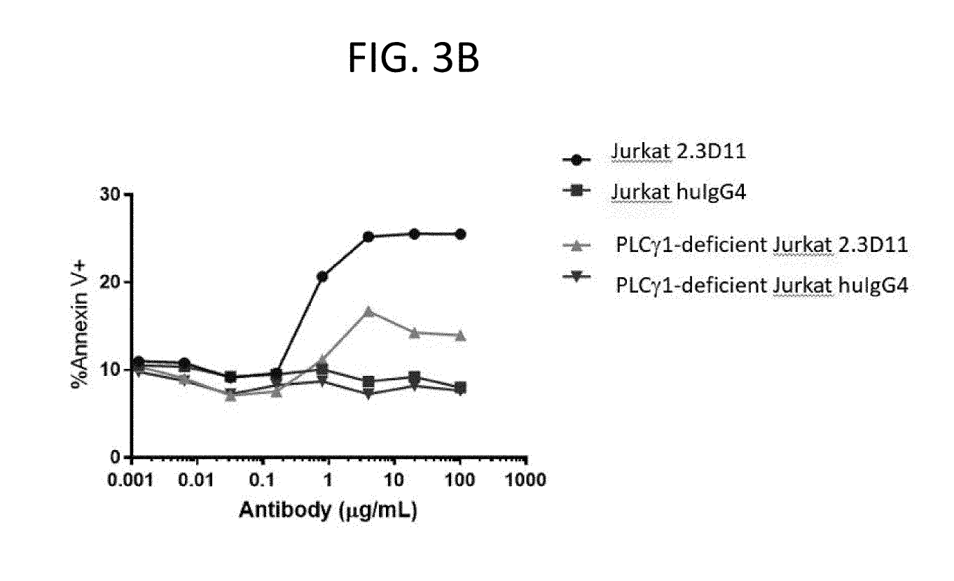

To assay whether anti-CD47 induced cell death was caspase dependent or independent Jurkat cells were pre-treated for 1 hour at 37° C., with DMSO (control), 100 μM pan-caspase inhibitor Z-VAD-FMK, 10 μM pan-caspase inhibitor Z-VAD-FMK, or 1 μM pan-caspase inhibitor Z-VAD-FMK. Cells were then added to plates containing immobilized isotype control antibody or immobilized antibody 2.3D11 and incubated for 24 hours. Cell death was evaluated by Annexin V+/Propidium Iodide (PI) staining via flow cytometry. As shown in FIG. 3A, the pan-caspase inhibitor had a negligible effect on immobilized antibody 2.3D11-mediated cell death.

To assay whether anti-CD47 induced cell death was PLCγ1 dependent or independent wildtype Jurkat cells or PLCγ1-deficient Jurkat cells (J.Gamma1 cells) were utilized. In particular, wildtype or PLCγ1-deficient Jurkat cells (J.Gamma1 cells) were exposed to a dose titration of immobilized antibody 2.3D11 or immobilized human IgG4 isotype antibody control, and incubated overnight in RPMI containing 10% FBS. Cell death was assessed by Annexin V surface staining by flow cytometry and the percent of events staining positive for Annexin V is graphed as a function of antibody concentration in FIG. 3B.

Results show that 2.3D11-induced cell death is partially dependent on PLCγ1 expression, and is caspase independent (FIGS. 3A and 3B).

### Example 3: Synergism of a Combination of Anti-CD47 Antibody and BCL-2 Inhibitor in a Human B Cell Lymphoma Model

To determine the anti-tumor efficacy of a combination of an anti-CD47 antibody and a cell death-inducing agent, the Ri-1 cell line was utilized. The Ri-1 cell line is a human B cell lymphoma line that represents the ABC-subtype of diffuse large cell B cell lymphoma (DLBCL) and harbors a BCL-2 amplification. ABT-199, also known as Venclexta™ or Venetoclax, is an oral BCL-2 inhibitor, and was used as the cell death-inducing agent. The anti-CD47 antibody was antibody 2.3D11 as described in Example 1.

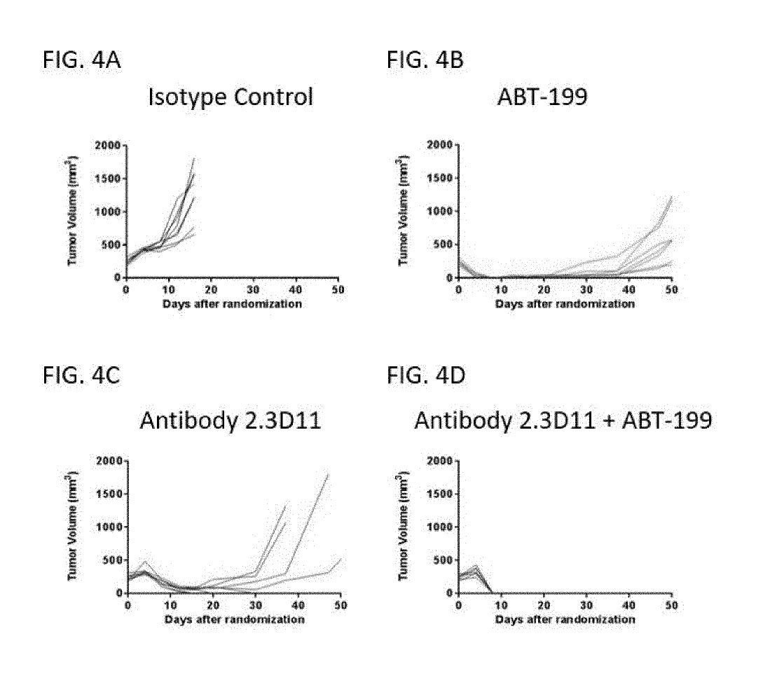

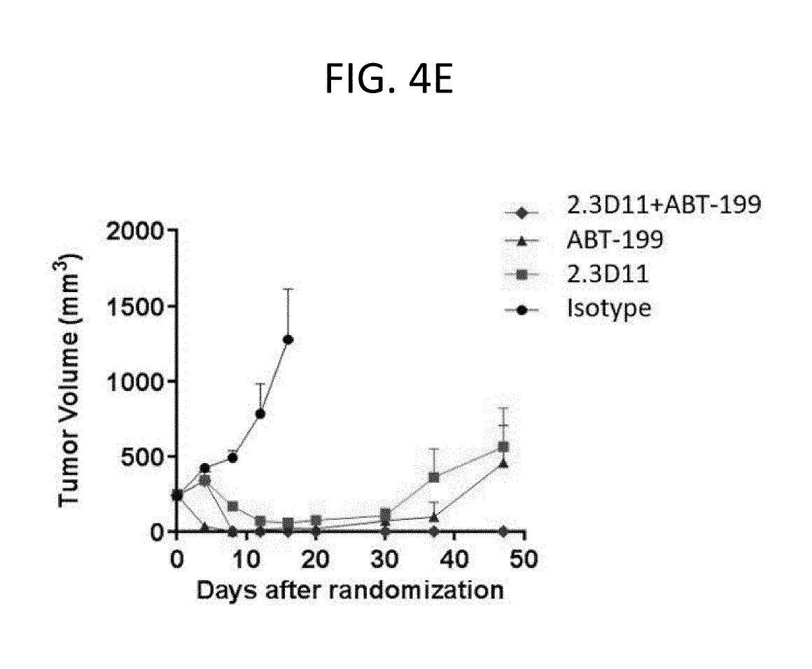

Briefly, 1×10⁷ Ri-1 cells were injected subcutaneously into the right flank of CB.17 SCID mice in a 1:1 mixture of RPMI and Matrigel. Tumors were allowed to grow until they were 200-300 mm³. Mice were then randomized to at least 7 mice per group and treated with either isotype antibody control, 2.3D11, ABT-199, or combination of 2.3D11 and ABT-199. 2.3D11 and the isotype control (polyclonal human IgG) were administered at 100 μg/mouse on days 0, 3 and 7 post randomization and ABT-199 was administered at 25 mg/kg on days 0, 1, 2, 3 and 4 post randomization. For the combination treatment, 2.3D11 was administered on day 0 (100 μg/mouse), followed by three daily, oral administrations of ABT-199 on days 3, 4 and 5 (25 mg/kg). FIGS. 4A-4D show the individual tumor volumes in mice with each treatment, and FIG. 4E shows mean tumor volume. 2.3D11 and ABT-199 alone induced potent single-agent anti-tumor activity. However, with both single agent treatments, tumors re-grew. The combination of 2.3D11 and ABT-199 prevented tumor re-growth. Moreover, these results show the effect of the combination when 2.3D11 treatment shortly preceded ABT-199. When the sequence of administration was reversed similar results occurred (data not shown). Overall, these results showed a synergistic effect on tumor re-growth when an antibody that specifically binds human CD47 (2.3D11) was administered in combination with a BCL-2 inhibitor (ABT-199).

### Example 4: Enhanced Efficacy of a Combination of Anti-CD47 Antibody and BCL-2 Inhibitor in Human AML Model with Reduced Sensitivity to ABT-199

The anti-tumor efficacy of a combination of an anti-CD47 antibody and a cell death inducing agent was assayed in a MOLM-13 xenograft model. The MOLM-13 tumor cell line is a human AML cell line that carries Internal tandem duplications (ITD) of the FLT3 gene and is wild-type for p53.

The cell death-inducing agent was ABT-199 as described in Example 3 and the anti-CD47 antibody was antibody 2.3D11 as described in Example 1.

Female NCI Athymic/nude mice (8-12 weeks old) were inoculated with 5×10⁶ MOLM-13 tumor cells in PBS: Matrigel, 1:1, subcutaneously in the flank. When tumors reached an average size of 50-80 mm³, animals were randomized into pair matched treatment groups (based on tumor size) with 10 mice per group, as denoted in Table 1 below:

TABLE 1
MOLM-13 xenograft model treatment regimens

| | | Regimen 1 | | | | Regimen 2 | | | |
|---|---|---|---|---|---|---|---|---|---|
| Gr. | N | Agent | μg/animal | Route | Schedule | Agent | μg/animal | Route | Schedule |
| 1^{#} | 10 | Isotype control | 60 | ip | biwk × 3 | vehicle | — | po | qd × 21 |
| | | (hIgG) | | | | | | | |
| 2 | 10 | 2.3D11 | 10 | ip | biwk × 3 | vehicle | — | po | qd × 21 |
| 3 | 10 | 2.3D11 | 60 | ip | biwk × 3 | vehicle | — | po | qd × 21 |
| 4 | 10 | Isotype control | 60 | ip | biwk × 3 | ABT-199 | 50* | po | qd × 21 |
| | | (hIgG) | | | | | | | |
| 5 | 10 | Isotype control | 60 | ip | biwk × 3 | ABT-199 | 100* | po | qd × 21 |
| | | (hIgG) | | | | | | | |
| 6 | 10 | 2.3D11 | 10 | ip | biwk × 3 | ABT-199 | 50* | po | qd × 21 |
| 7 | 10 | 2.3D11 | 10 | ip | biwk × 3 | ABT-199 | 100* | po | qd × 21 |
| 8 | 10 | 2.3D11 | 60 | ip | biwk × 3 | ABT-199 | 50* | po | qd × 21 |
| 9 | 10 | 2.3D11 | 60 | ip | biwk × 3 | ABT-199 | 100* | po | qd × 21 |

^{#}Control Group
*mg/kg

Dosing solutions were prepared fresh for each dose of 2.3D11 (PBS) and isotype antibody control (PBS). The dosing solution for ABT-199 (10% Ethanol/30% PEG400/60% Phosa150) was prepared once per week and stored at 4° C. The dosing volume for compounds administered by intraperitoneal (IP) injection was 0.2 mL/mouse, not adjusted for bodyweight. The dosing volume for compounds administered by oral administration (PO) was 10 mL/kg adjusted for body weight.

Animals were dosed according to the schedules for their treatment groups (Table 1). In particular, antibody 2.3D11 or isotype control (human polyclonal IgG) were administered IP twice weekly for three weeks, and ABT-199 was administered PO daily for 21 days.

Body Weight and tumor caliper measurements were recorded twice weekly from the time of randomization until the end of study. Animals were monitored individually. The endpoint of the experiment was a tumor volume of 2000 mm³ or 50 days, whichever came first. Animals responding to treatment were followed longer. When the endpoint was reached, the animals were euthanized.

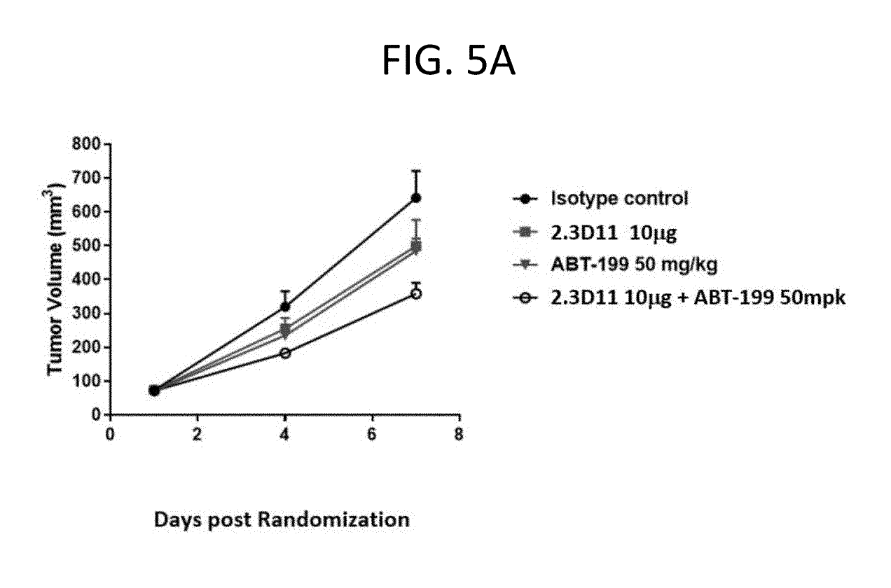

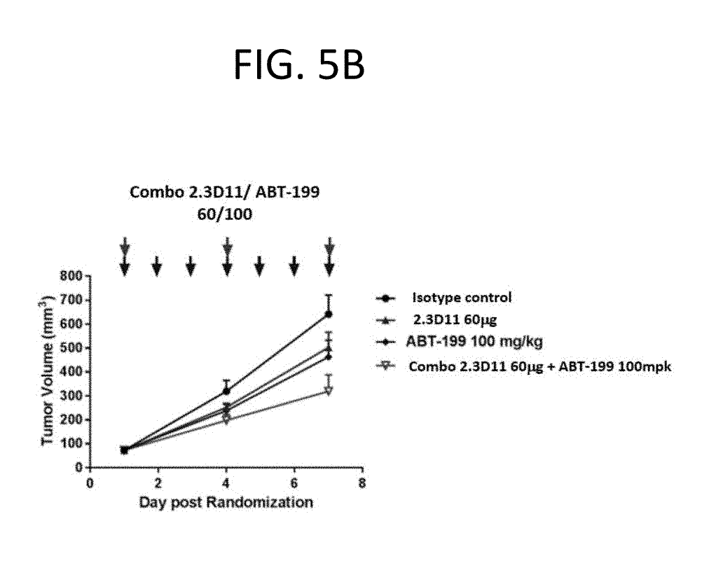

As shown in FIG. 5A and FIG. 5B, a combination of 2.3D11 with ABT-199 in MOLM-13 (AML) xenograft model enhances the anti-tumor activity over either agent alone at different dose combinations. In a MOLM-13 cell line with reduced sensitivity to ABT-199, and which expresses high levels of MCL-1, the combination of antibody 2.3D11 and ABT-199 results in an enhancement of anti-tumor activity without complete eradication of tumor.

### Example 5: Synergism of a Combination of an Anti-CD47 Antibody and BCL-2 Inhibitor in a ABT-199 Sensitive Human AML Model

The anti-tumor efficacy of a combination of an anti-CD47 antibody and a cell death inducing agent was assayed in a HL-60 xenograft model. The HL-60 tumor cell line is a human AML cell line, that is myc amplified and expresses lower levels of MCL-1 that the MOLM-13 cell line. The HL-60 cell line also carries a homozygous deletion of p53 and is wild-type for FLT3. The cell death-inducing agent was ABT-199 as described in Example 3 and the anti-CD47 antibody was antibody 2.3D11 as described in Example 1.

Female CB.17 SCID mice (8-12 weeks old) were injected with 5×10⁶ HL-60 cells, subcutaneously in the flank. When tumors reached an average size of 100-200 mm³, animals were randomized into pair matched treatment groups (based on tumor size) with ten animals per group, as denoted in Table 2 below:

TABLE 2
HL-60 xenograft model treatment regimens

| | # of | | Route of | Dose | |
|---|---|---|---|---|---|
| Group | Mice | Treatment | Adm. | Concentration | |
| 1 | 10 | Isotype control (hIgG) | IP | 100 | μg |
| 3 | 10 | 2.3D11 | IP | 30 | μg |
| 4 | 10 | ABT-199 | PO | 100 | mg/kg |

| | | | | |
|---|---|---|---|---|
| 6 | 10 | 2.3D11 + ABT-199 | IP | 30 μg + 100 mg/kg |

Dosing solutions were prepared fresh for each dose of 2.3D11 (PBS) and isotype antibody control (polyclonal human IgG) (PBS). The dosing solution for ABT-199 (10% Ethanol/30% PEG400/60% Phosa150) was prepared once per week and stored at 4° C. The dosing volume for compounds administered IP was 0.2 mL/mouse, not adjusted for bodyweight. The dosing volume for compounds administered PO was 10 mL/kg adjusted for body weight.

The dosing schedule for the animals in the different treatment groups was: Group 1 was administered the isotype control antibody (hIgG) by IP on days 0 and 3; Group 2 was administered antibody 2.3D11 by IP on days 0 and 3; Group 3 was administered ABT-199 orally on days 1, 2, 3, 4, and 5; and Group 4 was administered isotype control antibody and antibody 2.3D11 by IP on days 0 and 3 (see Table 2). Body Weight and tumor caliper measurements were recorded twice weekly from the time of randomization until the end of study. Animals were monitored individually. The endpoint of the experiment was a tumor volume of 2000 mm³ or 50 days, whichever came first.

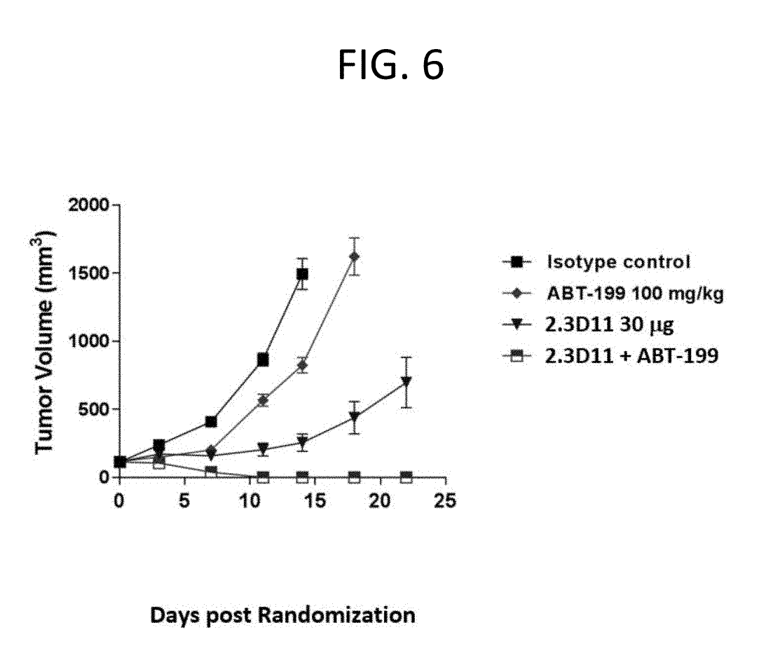

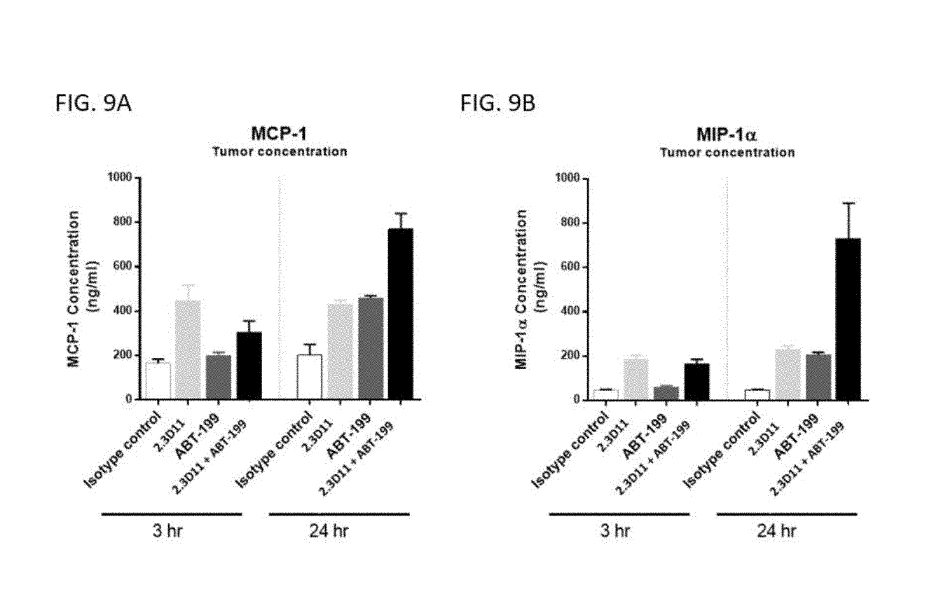

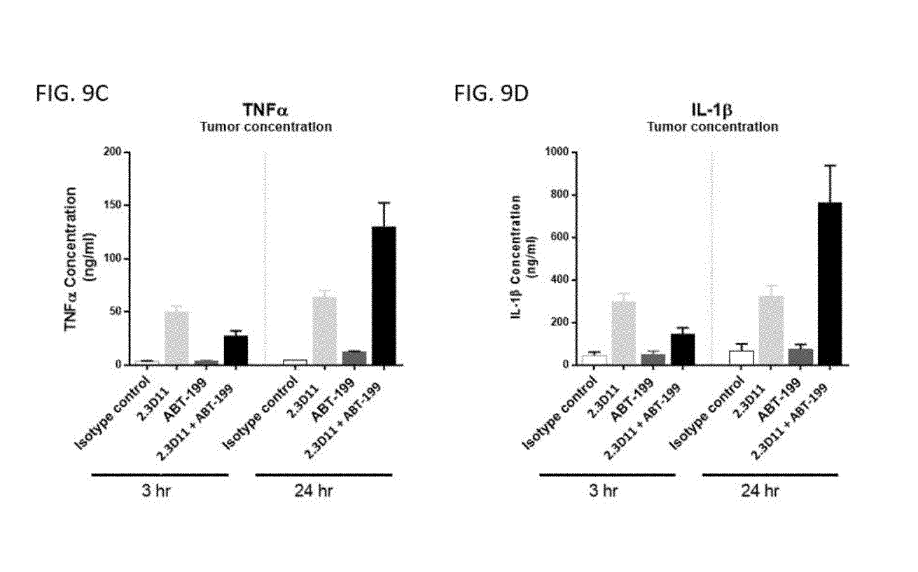

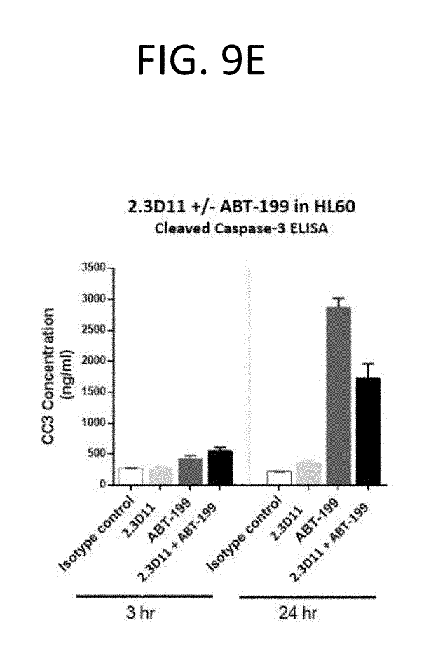

As shown in FIG. 6, a combination of 2.3D11 with ABT-199 shows synergistic efficacy in an HL-60 (AML) xenograft model. The data presented herein demonstrate that, in ABT-199-sensitive models, such as a Ri-1 xenograft model and a HL-60 xenograft model, the combination of antibody 2.3D11 and ABT-199 leads to a synergistic anti-tumor responsive. Without being bound by theory, it is believed that this synergy is the result of increased macrophage infiltration and clearance of dead and dying cells due to the ability of the combination of both agents to increase cytokines responsible for recruitment of macrophages (See FIG. 9).

### Example 6: Enhanced Efficacy of a Combination of Anti-CD47 Antibody and BCL-2 Inhibitor in an a Human Multiple Myeloma Model

The anti-tumor efficacy of a combination of an anti-CD47 antibody and a cell death inducing agent was assayed in a OPM2 xenograft model. The OMP2 tumor cell line is a human multiple myeloma cell line. The cell death-inducing agent was ABT-199 as described in Example 3 and the anti-CD47 antibody was antibody 2.3D11 as described in Example 1.

Female NCI CB.17 SCID mice (8-12 weeks old) were inoculated with 1×10⁷ OPM2 tumor cells in PBS:Matrigel, 1:1 subcutaneously in the flank. When tumors reached an average size of 100-150 mm³, animals were randomized into pair matched treatment groups (based on tumor size) with 10 animals per group, as denoted in Table 3 below:

TABLE 3
OPM2 xenograft model treatment regimens

| | | Regimen 1 | | | | Regimen 2 | | | |
|---|---|---|---|---|---|---|---|---|---|
| Gr. | N | Agent | μg/animal | Route | Schedule | Agent | μg/animal | Route | Schedule |
| 1 | 10 | Isotype | 30 | ip | biwk × 3 | saline | — | po | qd × 14 |
| | | Control hIgG | | | | | | | |
| 2 | 10 | 2.3D11 | 30 | ip | biwk × 3 | saline | — | po | qd × 14 |
| 3 | 10 | ABT-199 | 100* | po | qd × 14 | — | — | — | — |
| 5 | 10 | 2.3D11 | 30 | ip | biwk × 3 | ABT-199 | 100* | po | qd × 14 |

*mg/kg

Dosing solutions were prepared fresh for each dose of 2.3D11 (PBS) and isotype antibody control (PBS). The dosing solution for ABT-199 (10% Ethanol/30% PEG400/60% Phosa150) was prepared once per week and stored at 4° C. The dosing volume for compounds administered IP was 0.2 mL/mouse, not adjusted for bodyweight. The dosing volume for compounds administered PO was 10 mL/kg adjusted for body weight.

Animals were dosed according to the schedules for their treatment groups (Table 3). In particular, antibody 2.3D11 or isotype control (polyclonal human IgG) were administered IP twice weekly for three weeks and ABT-199 was administered PO daily for 14 days.

Body weight and tumor caliper measurements were recorded twice weekly from the time of randomization until the end of study. Animals were monitored individually. The endpoint of the experiment was a tumor volume of 2000 mm³ or 60 days, whichever came first. Animals responding to treatment were followed longer. When the endpoint was reached, the animals were euthanized.

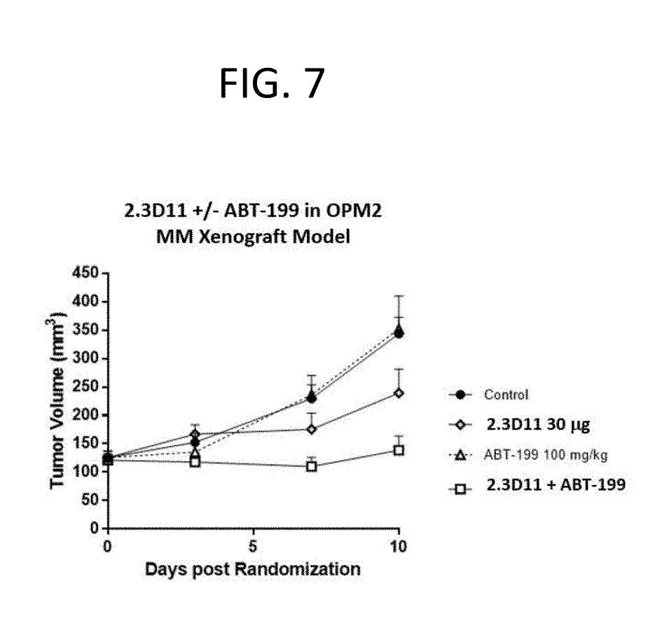

As shown in FIG. 7, a combination of 2.3D11 with ABT-199 shows enhanced efficacy in an OPM2 (multiple myeloma) xenograft model.

### Example 7: Enhanced Cell Death of a Combination of Anti-CD47 Antibody and BCL-2 Inhibitor in a Human AML Cell Line

The HL-60 cell line was used to assay the ability of the combination of an anti-CD47 antibody and a cell death inducing agent to induce cell death. The HL-60 cell line is a human AML cell line. The cell death-inducing agent was ABT-199 as described in Example 3 and the anti-CD47 antibody was antibody 2.3D11 as described in Example 1.

HL-60 cells were exposed to a dose titration of immobilized antibody 2.3D11 or immobilized isotype antibody control, with or without the addition of ABT-199 (10 nM), and incubated overnight at 37° C. The following day, cells were assayed by Flow Cytometry using Annexin V staining to assess cell death.

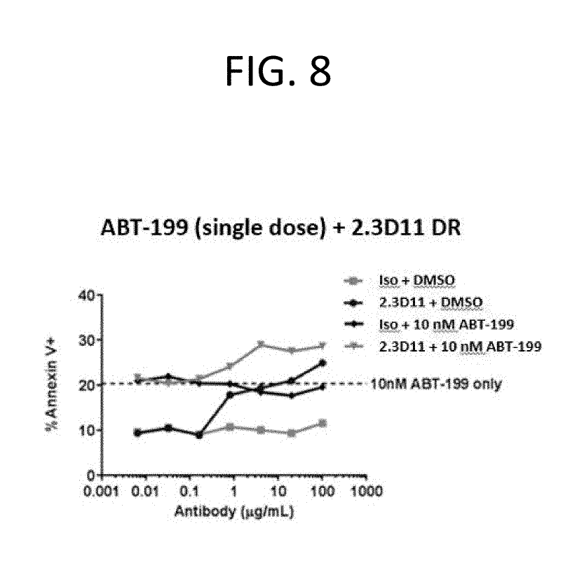

As shown in FIG. 8, the in vitro combination of ABT-199 and antibody 2.3D11 induces cell death in HL-60 Cells to a greater extent than either agent alone.

Similar results were observed when HL-60 cells were exposed to a single dose of immobilized antibody 2.3D11 (4 μg/ml) or immobilized isotype antibody control (4 μg/ml), and increasing concentrations of ABT-199 (0 nM, 0.01 nM, 0.1 nM, 1.0 nM, and 10 nM), and incubated overnight at 37° C. The following day, cells were assayed by Flow Cytometry using Annexin V staining to assess cell death (data not shown).

### Example 8: Combination of Anti-CD47 Antibody and BCL-2 Inhibitor Increases Tumor Levels of Pro-Inflammatory Chemokines and Cytokines

A potential mechanism of action for the combination of an anti-CD47 antibody and a cell death inducing agent was assessed in an HL-60 xenograft model. The HL-60 tumor cell line is a human AML cell line. The cell death-inducing agent was ABT-199 as described in Example 3 and the anti-CD47 antibody was antibody 2.3D11 as described in Example 1.

Female CB.17 SCID mice (8-12 weeks old) were injected with 5×10⁶ HL-60 cells, subcutaneously in the flank. When tumors reached an average size of 100-200 mm³, animals were randomized into pair matched treatment groups (based on tumor size) with ten animals per group, as denoted in Table 4 below.

TABLE 4
HL-60 xenograft model treatment regimens

| | | | | | | Collection |
|---|---|---|---|---|---|---|
| | | | | | Dose | Schedule |
| Group | Route | Treatment | # of Animals | Dose concentration | schedule | (hours) |
| 1 | IP | Isotype control (hIgG) | 10 | 100 ug | Single dose | 3, 24 |
| 2 | IP | 2.3D11 | 10 | 100 ug | Single dose | 3, 24 |
| 4 | PO | ABT-199 | 10 | 100 mg/kg | Single dose | 3, 24 |
| 6 | IP + PO | 2.3D11 + ABT-199 | 10 | 100 ug + 100 mg/kg | Single dose | 3, 24 |
| | | | | | Single dose | |

Dosing solutions were prepared fresh for each dose of 2.3D11 (PBS) and isotype antibody control (polyclonal human IgG in PBS). The dosing solution for ABT-199 (10% Ethanol/30% PEG400/60% Phosa150) was prepared once per week and stored at 4° C. The dosing volume for compounds administered IP was 0.2 mL/mouse, not adjusted for bodyweight.

The dosing volume for compounds administered PO was 10 mL/kg adjusted for body weight. Animals were dosed according to the schedules for their treatment groups as shown in Table 4. Five mice from each group were euthanized at 3 and 24 hours after the last injection, tumor tissue was collected and homogenized in RIPA lysis buffer+phosphatase and protease inhibitors.

MCP-1, MIP-1α, TNFα, and IL-1β cytokine concentrations in the tumor lysate were determined with the V-PLEX™ Mouse (Meso Scale Discovery) kit according to manufacturer's instructions. Apoptosis was measured using the Pathscan Cleaved Caspase-3 Sandwich ELISA Kit (Cell Signaling Technology).

As shown in FIGS. 9A and 9B, a combination of 2.3D11 with ABT-199 synergizes and significantly increases the levels of the monocyte chemoattractant protein 1 (MCP-1) and macrophage inflammatory protein 1-alpha (MIP-1α) at 24 hours. Both chemokines are known to elicit an inflammatory response that leads to macrophage infiltration into the tumor. In FIGS. 9C and 9D, upregulation of TNFα and IL-1β is shown suggesting the presence of immune cell infiltration. In contrast to monotherapy, and unexpectedly, the level of cleaved caspase-3 was significantly lower in the combination of 2.3D11 and ABT-199 at 24 hours FIG. 9E, indicating caspase-dependent cell death. Without being bound by theory, it is believed that these data together with presence of elevated MCP-1 and MIP-1α suggest an acceleration of the clearance mechanism, likely due to the presence of recruited macrophages in the tumor.

### Example 9: Enhanced Cell Death of a Combination of Anti-CD47 Antibody and an Immunogenic Cell Death Inducing Agent

The effectiveness of a combination of an anti-CD47 antibody and one of 3 different immunogenic cell death (ICD) inducing agents to induce cell death was determined. Specifically, the ability of a combination of an anti-CD47 antibody with an anthracycline (doxorubicin), a platinum derivative (oxaliplatin) or a proteasome inhibitor (Bortezomib) to induce cell death was determined.

Jurkat cells (malignant human T cells) were utilized to assay the ability of an anti-CD47 antibody in combination with Doxorubicin to induce cell death. Doxorubicin has been shown to induce ICD and is a calreticulin inducing agent in the anthracycline family and has been used to treat ovarian cancer. The anti-CD47 antibody was antibody 2.3D11 as described in Example 1.

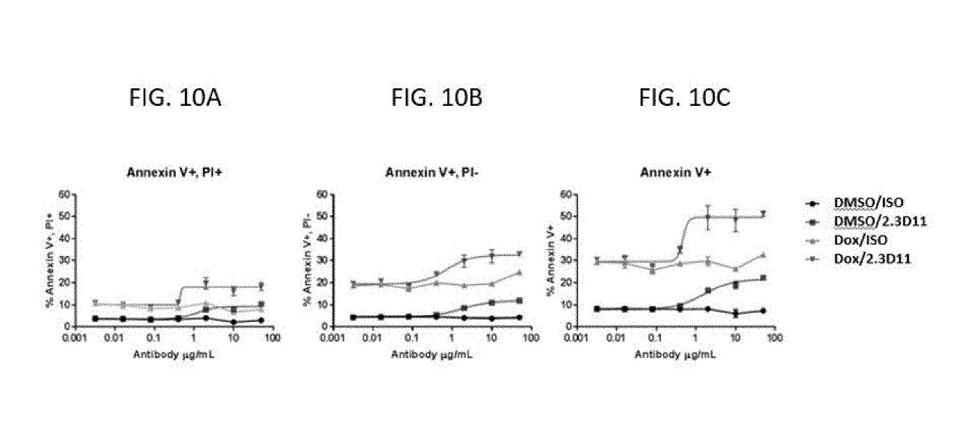

As shown in FIGS. 10A-C Jurkat cells were cultured with immobilized antibody (isotype control or antibody 2.3D11), 150 nM doxorubicin, or a combination of immobilized antibody (isotype control or antibody 2.3D11) and 150 nM doxorubicin. Cells were incubated overnight at 37° C., 5% CO₂ and cell death was evaluated by Annexin V/PI staining via flow cytometry. Necrotic cells show positive staining for both Annexin V and PI (FIG. 10A) while apoptotic cells are Annexin V positive and PI negative (FIG. 10B). Apoptotic and necrotic cells were identified by total AnnexinV+staining (FIG. 10C).

These data show that the combination of immobilized antibody 2.3D11 and doxorubicin enhances Jurkat cell death over either agent individually.

The ability an anti-CD47 antibody and oxaliplatin to induce cell death was assayed in Jurkat cells. Oxaliplatin is a platinum based chemotherapy drug that has been shown to induce ICD and cell surface calreticulin. The anti-CD47 antibody was antibody 2.3D11 as described in Example 1.

Plates were pre-coated with a single dose of immobilized antibody 2.3D11 (1 μg/ml) or immobilized isotype antibody control (IgG4) (1 μg/ml), and incubated overnight at 4° C. Jurkat cells were seeded on the pre-coated plates with a dose titration of oxaliplatin and incubated overnight at 37° C. The following day, Jurkat cell death evaluated by AnnexinV/PI staining via flow cytometry. Necrotic cells show positive staining for both AnnexinV and PI; apoptotic cells are positive for AnnexinV staining and negative for PI staining; and apoptotic and necrotic cells were identified by total AnnexinV+staining.

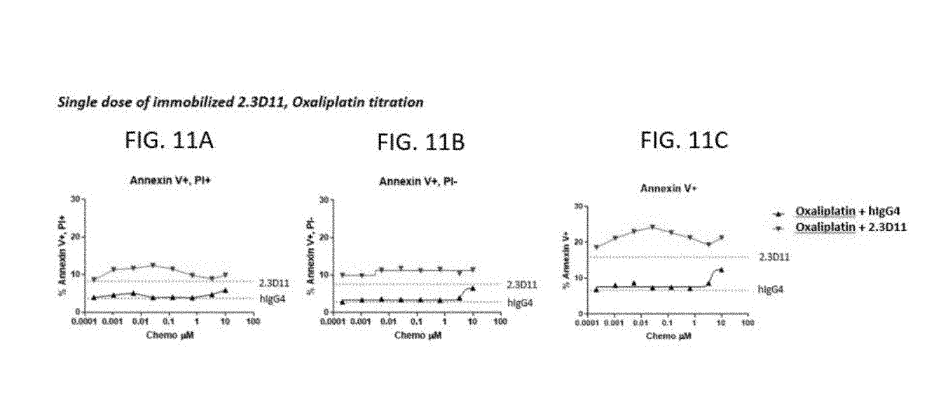

As shown in FIG. 11A-C, the combination of oxaliplatin and antibody 2.3D11 induced cell death in Jurkat cells to a greater extent than oxaliplatin in combination with an isotype antibody control. Oxaliplatin has been also shown to increase MHC I expression and DC maturation and these effects may be potentiated or overall immune potentiating effects of platinum drugs may be enhanced when combined with antibody 2.3D11.

The ability an anti-CD47 antibody and bortezomib to induce cell death was assayed in Jurkat cells. Bortezomib is a proteasome inhibitor which is known to induce ICD and surface calreticulin. The anti-CD47 antibody was antibody 2.3D11 as described in Example 1.

Plates were pre-coated with a single dose of immobilized antibody 2.3D11 (1 μg/ml) or immobilized isotype antibody control (IgG4) (1 μg/ml), and incubated overnight at 4° C. Jurkat cells were seeded on the pre-coated plates with a dose titration of bortezomib and incubated overnight at 37° C. The following day, Jurkat cell death evaluated by AnnexinV/PI staining via flow cytometry. Necrotic cells show positive staining for both AnnexinV and PI; apoptotic cells are positive for AnnexinV staining and negative for PI staining; and apoptotic and necrotic cells were identified by total AnnexinV+staining.

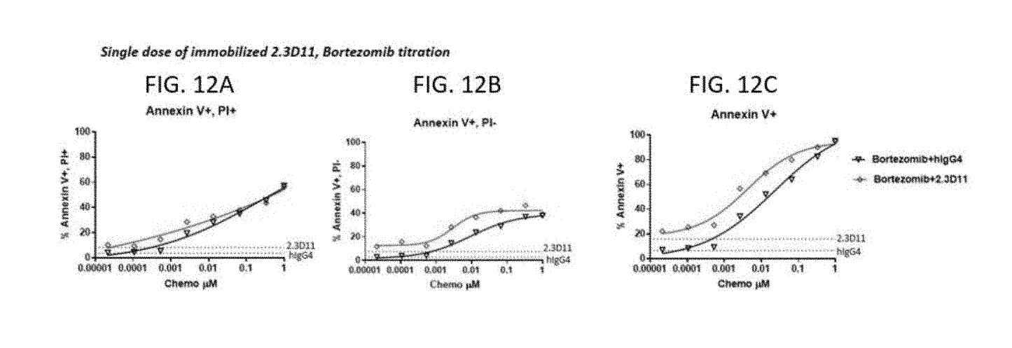

As shown in FIG. 12A-C, the combination of bortezomib and antibody 2.3D11 enhanced cell death in Jurkat cells to a greater extent than bortezomib in combination with an isotype antibody control.

### Example 10: Enhanced Efficacy with a Combination of Anti-CD47 Antibody and Proteasome Inhibitor in a Human Multiple Myeloma Model

The anti-tumor efficacy of a combination of an anti-CD47 antibody and a proteasome inhibitor was assayed in a OPM2 xenograft model. The OMP2 tumor cell line is a human multiple myeloma cell line. Bortezomib, also known as Velcade™, was used as a proteasome inhibitor. Velcade can also induce cell death by upregulation of NOXA, a pro-apoptotic BH3 only protein. In addition, Velcade can trigger Mcl-1 cleavage in Mcl-1(wt/wt), caspase-3 and -9, and PARP cleavage. The anti-CD47 antibody was antibody 2.3D11 as described in Example 1. Female NCI CB.17 SCID mice (8-12 weeks old) were inoculated with 1×10⁷ OPM2 tumor cells in PBS:Matrigel, 1:1 subcutaneously in the flank. When tumors reached an average size of 100-150 mm³, animals were randomized into pair matched treatment groups (based on tumor size) with 10 animals per group, as denoted in Table 5 below:

TABLE 5
OPM2 xenograft model treatment regimens

| | | Regimen 1 | | | | Regimen 2 | | | |
|---|---|---|---|---|---|---|---|---|---|
| Gr. | N | Agent | μg/animal | Route | Schedule | Agent | μg/animal | Route | Schedule |
| 1 | 10 | Isotype | 30 | ip | biwk × 3 | saline | — | po | qd × 14 |
| | | Control hIgG | | | | | | | |
| 2 | 10 | 2.3D11 | 30 | ip | biwk × 3 | saline | — | po | qd × 14 |
| 4 | 10 | bortezomib | 0.5* | iv | days 1, 5, 9 | — | — | — | — |
| 6 | 10 | 2.3D11 | 30 | ip | biwk × 3 | bortezomib | 0.5* | iv | days 1, 5, 9 |

*mg/kg

Dosing solutions were prepared fresh for each dose of 2.3D11 (PBS) and isotype antibody control (PBS). Velcade in saline was acquired commercially and stored per manufacturer's instructions. The dosing volume for compounds administered IP was 0.2 mL/mouse, not adjusted for bodyweight. The dosing volume for compounds administered by intravenous (IV) administration was 5 mL/Kg.

Animals were dosed according to the schedules for their treatment groups (Table 5). In particular, antibody 2.3D11 or isotype control (polyclonal human IgG) were administered IP twice weekly for three weeks and bortezomib was administered IV on days 1, 5, and 9.

Body weight and tumor caliper measurements were recorded twice weekly from the time of randomization until the end of study. Animals were monitored individually. The endpoint of the experiment was a tumor volume of 2000 mm³ or 60 days, whichever came first. Animals responding to treatment were followed longer. When the endpoint was reached, the animals were euthanized.

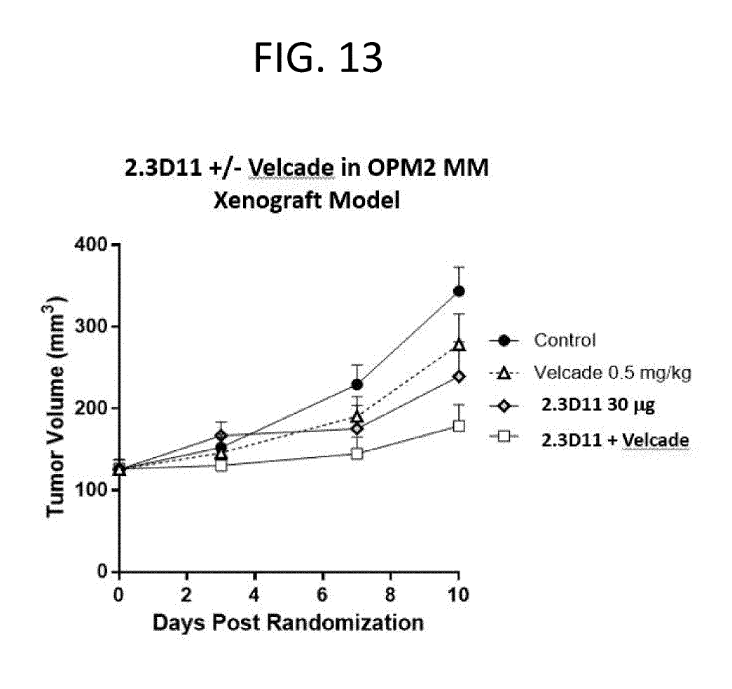

As shown in FIG. 13, a combination of 2.3D11 with Velcade in an OPM2 (multiple myeloma) xenograft model enhanced the anti-tumor efficacy over either agent alone.

### Example 11: Prolonged Survival with a Combination of Anti-CD47 Antibody and PARP Inhibitor

The ability of a combination of an anti-CD47 antibody and a PARP inhibitor to prolong survival was assayed in a SKOV-3-GFP xenograft model. The SKOV-3 tumor cell line is a human ovarian cancer cell line. Olaparib was used as a PARP inhibitor. The anti-CD47 antibody was antibody 2.3D11 as described in Example 1.

Female CB.17 SCID mice (7-8 weeks old) were purchased from the Charles River Laboratories and acclimated for 1 week. The mice were caged in groups of five in an air-filtered laminar flow cabinet. All procedures were performed under sterile conditions in a laminar flow hood. Animal experiments were conducted under guidelines approved by the Institutional Animal Care and Use Committee (IACUC). Human ovarian cancer cells, SKOV-3-GFP, were injected into the abdominal cavities of SCID mice using 26-gauge needle. Each mouse received 5×10⁶ cells in 100 μl PBS. On day 24 post implant mice were randomized into four groups, with seven or ten mice per group, based on the green fluorescence signal (GFP) using IVIS Imaging System (Xenogen, Alameda, Calif., USA); isotype antibody control; 2.3D11, Olaparib, or combination of 2.3D11 and Olaparib, as denoted in Table 6 below:

TABLE 6
SKOV-3-GFP intraperitoneal xenograft model treatment regimens

| | # of | | Route of | | |
|---|---|---|---|---|---|
| Gr. | Mice | Treatment | administration | Dose Conc. | Schedule |
| 1 | 10 | Isotype | I.P* | 100 ug/mouse | Day 0, 7, 14 and day 28 |
| 2 | 7 | 2.3D11 | I.P | 100 ug/mouse | Day 0, 7, 14 and day 28 |
| 3 | 7 | PARP (Olaparib) | P.O* | 50 mg/kg | Day 7, 8, 9, 28, 29 and day |
| | | | | | 30 |

| | | | | |
|---|---|---|---|---|
| 4 | 7 | 2.3D11 + Olaparib | I.P + P.O | Dose and schedule was performed same as |
| | | | | Monotherapy as indicated in rows 2 & 3 |
| | | | | respectively |

*I.P: Intraperotonial
*P.O: Peroral

Dosing solutions were prepared fresh for each dose of 2.3D11, isotype antibody control, and Olaparib (PBS). Dosing volume for compounds administered IP was 0.2 mL/mouse, not adjusted for bodyweight. Dosing volume for compounds administered PO was 10 mL/kg. Animals were dosed according to the schedules for their treatment groups (Table 6).

Mice were monitored twice weekly for body weight and clinical observations including increasing volume of ascites, defined by the dark coloration of the abdomen due to hemorrhagic ascites. Animals were monitored individually. The endpoint of the experiment was loss of body weight greater than 20% or hemorrhagic ascites. Animals responding to treatment were followed for a maximum of 200 days. When the endpoint was reached, the animals were euthanized.

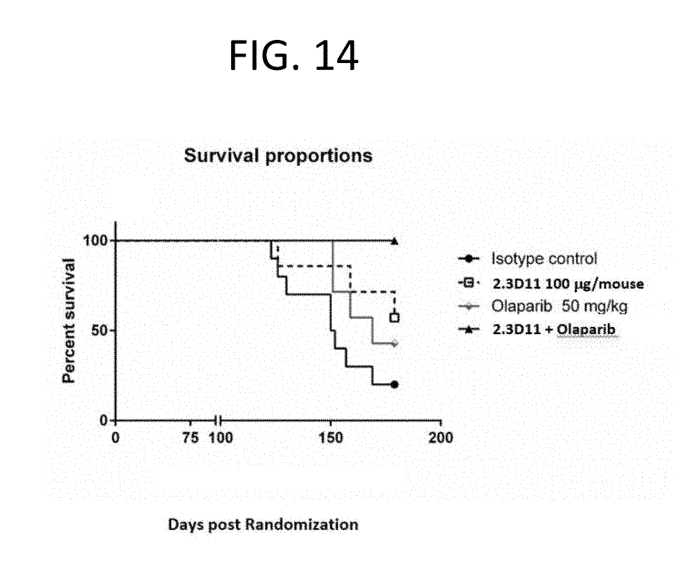

As shown in FIG. 14, a combination of 2.3D11 and PARP inhibitor (Olaparib) extended the survival of mice bearing SKOV-3-GFP intraperitoneal xenograft over each agent used as monotherapy.

SUMMARY OF SEQUENCES

| SEQ ID | | |
|---|---|---|
| NO | Description | Sequence |
| 1 | Human CD47 | MWPLVAALLLGSACCGSAQLLFNKTKSVEFTFCNDTVVIP |
| | protein (NCBI | CFVTNMEAQNTTEVYVKWKFKGRDIYTFDGALNKSTVPT |
| | Ref Sequence: | DFSSAKIEVSQLLKGDASLKMDKSDAVSHTGNYTCEVTEL |
| | NP_001768.1) | TREGETIIELKYRVVSWFSPNENILIVIFPIFAILLFWGQFGIK |
| | | TLKYRSGGMDEKTIALLVAGLVITVIVIVGAILFVPGEYSL |
| | | KNATGLGLIVTSTGILILLHYYVFSTAIGLTSFVIAILVIQVI |
| | | AYILAVVGLSLCIAACIPMHGPLLISGLSILALAQLLGLVY |
| | | MKFVASNQKTIQPPRKAVEEPLNAFKESKGMMNDE |
| 2 | Human CD47 | GGGGAGCAGGCGGGGGAGCGGGCGGGAAGCAGTGGGA |
| | mRNA (NCBI | GCGCGCGTGCGCGCGGCCGTGCAGCCTGGGCAGTGGGT |
| | Ref Sequence: | CCTGCCTGTGACGCGCGGCGGCGGTCGGTCCTGCCTGT |
| | NP_001768.1) | AACGGCGGCGGCGGCTGCTGCTCCAGACACCTGCGGCG |
| | | GCGGCGGCGACCCCGCGGCGGGCGCGGAGATGTGGCCC |
| | | CTGGTAGCGGCGCTGTTGCTGGGCTCGGCGTGCTGCGG |
| | | ATCAGCTCAGCTACTATTTAATAAAACAAAATCTGTAG |
| | | AATTCACGTTTTGTAATGACACTGTCGTCATTCCATGCT |
| | | TTGTTACTAATATGGAGGCACAAAACACTACTGAAGTA |
| | | TACGTAAAGTGGAAATTTAAAGGAAGAGATATTTACAC |
| | | CTTTGATGGAGCTCTAAACAAGTCCACTGTCCCCACTGA |
| | | CTTTAGTAGTGCAAAAATTGAAGTCTCACAATTACTAA |
| | | AAGGAGATGCCTCTTTGAAGATGGATAAGAGTGATGCT |
| | | GTCTCACACACAGGAAACTACACTTGTGAAGTAACAGA |
| | | ATTAACCAGAGAAGGTGAAACGATCATCGAGCTAAAAT |
| | | ATCGTGTTGTTTCATGGTTTTCTCCAAATGAAAATATTC |
| | | TTATTGTTATTTTCCCAATTTTTGCTATACTCCTGTTCTG |
| | | GGGACAGTTTGGTATTAAAACACTTAAATATAGATCCG |
| | | GTGGTATGGATGAGAAAACAATTGCTTTACTTGTTGCTG |
| | | GACTAGTGATCACTGTCATTGTCATTGTTGGAGCCATTC |
| | | TTTTCGTCCCAGGTGAATATTCATTAAAGAATGCTACTG |
| | | GCCTTGGTTTAATTGTGACTTCTACAGGGATATTAATAT |
| | | TACTTCACTACTATGTGTTTAGTACAGCGATTGGATTAA |
| | | CCTCCTTCGTCATTGCCATATTGGTTATTCAGGTGATAG |
| | | CCTATATCCTCGCTGTGGTTGGACTGAGTCTCTGTATTG |
| | | CGGCGTGTATACCAATGCATGGCCCTCTTCTGATTTCAG |
| | | GTTTGAGTATCTTAGCTCTAGCACAATTACTTGGACTAG |
| | | TTTATATGAAATTTGTGGCTTCCAATCAGAAGACTATAC |
| | | AACCTCCTAGGAAGCTGTAGAGGAACCCCTTAATGCAT |
| | | TCAAAGAATCAAAAGGAATGATGAATGATGAATAACTG |
| | | AAGTGAAGTGATGGACTCCGATTTGGAGAGTAGTAAGA |
| | | CGTGAAAGGAATACACTTGTGTTTAAGCACCATGGCCT |
| | | TGATGATTCACTGTTGGGGAGAAGAAACAAGAAAAGTA |
| | | ACTGGTTGTCACCTATGAGACCCTTACGTGATTGTTAGT |
| | | TAAGTTTTTATTCAAAGCAGCTGTAATTTAGTTAATAAA |
| | | ATAATTATGATCTATGTTGTTTGCCCAATTGAGATCCAG |
| | | TTTTTTGTTGTTATTTTTAATCAATTAGGGGCAATAGTA |
| | | GAATGGACAATTTCCAAGAATGATGCCTTTCAGGTCCT |
| | | AGGGCCTCTGGCCTCTAGGTAACCAGTTTAAATTGGTTC |
| | | AGGGTGATAACTACTTAGCACTGCCCTGGTGATTACCC |
| | | AGAGATATCTATGAAAACCAGTGGCTTCCATCAAACCT |
| | | TTGCCAACTCAGGTTCACAGCAGCTTTGGGCAGTTATGG |
| | | CAGTATGGCATTAGCTGAGAGGTGTCTGCCACTTCTGG |
| | | GTCAATGGAATAATAAATTAAGTACAGGCAGGAATTTG |
| | | GTTGGGAGCATCTTGTATGATCTCCGTATGATGTGATAT |
| | | TGATGGAGATAGTGGTCCTCATTCTTGGGGGTTGCCATT |
| | | CCCACATTCCCCCTTCAACAAACAGTGTAACAGGTCCTT |
| | | CCCAGATTTAGGGTACTTTTATTGATGGATATGTTTTCC |
| | | TTTTATTCACATAACCCCTTGAAACCCTGTCTTGTCCTCC |
| | | TGTTACTTGCTTCTGCTGTACAAGATGTAGCACCTTTTC |
| | | TCCTCTTTGAACATGGTCTAGTGACACGGTAGCACCAGT |
| | | TGCAGGAAGGAGCCAGACTTGTTCTCAGAGCACTGTGT |
| | | TCACACTTTTCAGCAAAAATAGCTATGGTTGTAACATAT |
| | | GTATTCCCTTCCTCTGATTTGAAGGCAAAAATCTACAGT |
| | | GTTTCTTCACTTCTTTTCTGATCTGGGGCATGAAAAAAG |
| | | CAAGATTGAAATTTGAACTATGAGTCTCCTGCATGGCA |
| | | ACAAAATGTGTGTCACCATCAGGCCAACAGGCCAGCCC |
| | | TTGAATGGGGATTTATTACTGTTGTATCTATGTTGCATG |
| | | ATAAACATTCATCACCTTCCTCCTGTAGTCCTGCCTCGT |
| | | ACTCCCCTTCCCCTATGATTGAAAAGTAAACAAAACCC |
| | | ACATTTCCTATCCTGGTTAGAAGAAAATTAATGTTCTGA |
| | | CAGTTGTGATCGCCTGGAGTACTTTTAGACTTTTAGCAT |
| | | TCGTTTTTTACCTGTTTGTGGATGTGTGTTTGTATGTGCA |
| | | TACGTATGAGATAGGCACATGCATCTTCTGTATGGACA |
| | | AAGGTGGGGTACCTACAGGAGAGCAAAGGTTAATTTTG |
| | | TGCTTTTAGTAAAAACATTTAAATACAAAGTTCTTTATT |
| | | GGGTGGAATTATATTTGATGCAAATATTTGATCACTTAA |
| | | AACTTTTAAAACTTCTAGGTAATTTGCCACGCTTTTTGA |
| | | CTGCTCACCAATACCCTGTAAAAATACGTAATTCTTCCT |
| | | GTTTGTGTAATAAGATATTCATATTTGTAGTTGCATTAA |
| | | TAATAGTTATTTCTTAGTCCATCAGATGTTCCCGTGTGC |
| | | CTCTTTTATGCCAAATTGATTGTCATATTTCATGTTGGG |
| | | ACCAAGTAGTTTGCCCATGGCAAACCTAAATTTATGAC |
| | | CTGCTGAGGCCTCTCAGAAAACTGAGCATACTAGCAAG |
| | | ACAGCTCTTCTTGAAAAAAAAAATATGTATACACAAAT |
| | | ATATACGTATATCTATATATACGTATGTATATACACACA |
| | | TGTATATTCTTCCTTGATTGTGTAGCTGTCCAAAATAAT |
| | | AACATATATAGAGGGAGCTGTATTCCTTTATACAAATCT |
| | | GATGGCTCCTGCAGCACTTTTTCCTTCTGAAAATATTTA |
| | | CATTTTGCTAACCTAGTTTGTTACTTTAAAAATCAGTTTT |
| | | GATGAAAGGAGGGAAAAGCAGATGGACTTGAAAAAGA |
| | | TCCAAGCTCCTATTAGAAAAGGTATGAAAATCTTTATA |
| | | GTAAAATTTTTTATAAACTAAAGTTGTACCTTTTAATAT |
| | | GTAGTAAACTCTCATTTATTTGGGGTTCGCTCTTGGATC |
| | | TCATCCATCCATTGTGTTCTCTTTAATGCTGCCTGCCTTT |
| | | TGAGGCATTCACTGCCCTAGACAATGCCACCAGAGATA |
| | | GTGGGGGAAATGCCAGATGAAACCAACTCTTGCTCTCA |
| | | CTAGTTGTCAGCTTCTCTGGATAAGTGACCACAGAAGC |
| | | AGGAGTCCTCCTGCTTGGGCATCATTGGGCCAGTTCCTT |
| | | CTCTTTAAATCAGATTTGTAATGGCTCCCAAATTCCATC |
| | | ACATCACATTTAAATTGCAGACAGTGTTTTGCACATCAT |
| | | GTATCTGTTTTGTCCCATAATATGCTTTTTACTCCCTGAT |
| | | CCCAGTTTCTGCTGTTGACTCTTCCATTCAGTTTTATTTA |
| | | TTGTGTGTTCTCACAGTGACACCATTTGTCCTTTTCTGCA |
| | | ACAACCTTTCCAGCTACTTTTGCCAAATTCTATTTGTCTT |
| | | CTCCTTCAAAACATTCTCCTTTGCAGTTCCTCTTCATCTG |
| | | TGTAGCTGCTCTTTTGTCTCTTAACTTACCATTCCTATAG |
| | | TACTTTATGCATCTCTGCTTAGTTCTATTAGTTTTTTGGC |
| | | CTTGCTCTTCTCCTTGATTTTAAAATTCCTTCTATAGCTA |
| | | GAGCTTTTCTTTCTTTCATTCTCTCTTCCTGCAGTGTTTT |
| | | GCATACATCAGAAGCTAGGTACATAAGTTAAATGATTG |
| | | AGAGTTGGCTGTATTTAGATTTATCACTTTTTAATAGGG |
| | | TGAGCTTGAGAGTTTTCTTTCTTTCTGTTTTTTTTTTTTGT |
| | | TTTTTTTTTTTTTTTTTTTTTTTTTTTTTTTGACTAATTTCA |
| | | CATGCTCTAAAAACCTTCAAAGGTGATTATTTTTCTCCT |
| | | GGAAACTCCAGGTCCATTCTGTTTAAATCCCTAAGAATG |
| | | TCAGAATTAAAATAACAGGGCTATCCCGTAATTGGAAA |
| | | TATTTCTTTTTTCAGGATGCTATAGTCAATTTAGTAAGT |
| | | GACCACCAAATTGTTATTTGCACTAACAAAGCTCAAAA |
| | | CACGATAAGTTTACTCCTCCATCTCAGTAATAAAAATTA |
| | | AGCTGTAATCAACCTTCTAGGTTTCTCTTGTCTTAAAAT |
| | | GGGTATTCAAAAATGGGGATCTGTGGTGTATGTATGGA |
| | | AACACATACTCCTTAATTTACCTGTTGTTGGAAACTGGA |
| | | GAAATGATTGTCGGGCAACCGTTTATTTTTTATTGTATT |
| | | TTATTTGGTTGAGGGATTTTTTTATAAACAGTTTTACTTG |
| | | TGTCATATTTTAAAATTACTAACTGCCATCACCTGCTGG |
| | | GGTCCTTTGTTAGGTCATTTTCAGTGACTAATAGGGATA |
| | | ATCCAGGTAACTTTGAAGAGATGAGCAGTGAGTGACCA |
| | | GGCAGTTTTTCTGCCTTTAGCTTTGACAGTTCTTAATTA |
| | | AGATCATTGAAGACCAGCTTTCTCATAAATTTCTCTTTT |
| | | TGAAAAAAAGAAAGCATTTGTACTAAGCTCCTCTGTAA |
| | | GACAACATCTTAAATCTTAAAAGTGTTGTTATCATGACT |
| | | GGTGAGAGAAGAAAACATTTTGTTTTTATTAAATGGAG |
| | | CATTATTTACAAAAAGCCATTGTTGAGAATTAGATCCCA |
| | | CATCGTATAAATATCTATTAACCATTCTAAATAAAGAG |
| | | AACTCCAGTGTTGCTATGTGCAAGATCCTCTCTTGGAGC |
| | | TTTTTTGCATAGCAATTAAAGGTGTGCTATTTGTCAGTA |
| | | GCCATTTTTTTGCAGTGATTTGAAGACCAAAGTTGTTTT |
| | | ACAGCTGTGTTACCGTTAAAGGTTTTTTTTTTTATATGTA |
| | | TTAAATCAATTTATCACTGTTTAAAGCTTTGAATATCTG |
| | | CAATCTTTGCCAAGGTACTTTTTTATTTAAAAAAAAACA |
| | | TAACTTTGTAAATATTACCCTGTAATATTATATATACTT |
| | | AATAAAACATTTTAAGCTATTTTGTTGGGCTATTTCTAT |
| | | TGCTGCTACAGCAGACCACAAGCACATTTCTGAAAAAT |
| | | TTAATTTATTAATGTATTTTTAAGTTGCTTATATTCTAGG |
| | | TAACAATGTAAAGAATGATTTAAAATATTAATTATGAA |
| | | TTTTTTGAGTATAATACCCAATAAGCTTTTAATTAGAGC |
| | | AGAGTTTTAATTAAAAGTTTTAAATCAGTC |
| 3 | 2.3D11 VH | QVQLQESGPGLVKPSGTLSLTCAVSGVSIRSINWWNWVR |
| | | QPPGKGLEWIGEIYHSGSTNYNPSLKSRVTISVDKSKNQFS |
| | | LKLNSVTAADTAVYYCARDGGIAVTDYYYYGLDVWGQG |
| | | TTVTVSS |
| 4 | 2.3D11 VL | EIVLTQSPATLSLSPGERATLSCRASESVSSNLAWYQQKPG |
| | | QAPRLLIYGAFNRATGIPARFSGSGSGTDFTLTISSLEPEDF |
| | | AVYYCQQRSDWFTFGGGTKVEIK |
| 5 | 2.3D11 | SINWWN |
| | HCDR1 | |
| 6 | 2.3D11 | EIYHSGSTNYNPSLKS |
| | HCDR2 | |
| 7 | 2.3D11 | DGGIAVTDYYYYGLDV |
| | HCDR3 | |
| 8 | 2.3D11 | RASESVSSNLA |
| | LCDR1 | |
| 9 | 2.3D11 | GAFNRAT |
| | LCDR2 | |
| 10 | 2.3D11 | QQRSDWFT |
| | LCDR3 | |
| 11 | 2.3D11 heavy | QVQLQESGPGLVKPSGTLSLTCAVSGVSIRSINWWNWVR |
| | chain | QPPGKGLEWIGEIYHSGSTNYNPSLKSRVTISVDKSKNQFS |
| | | LKLNSVTAADTAVYYCARDGGIAVTDYYYYGLDVWGQG |
| | | TTVTVSSAETTAPSVYPLAPGTALKSNSMVTLGCLVKGYF |
| | | PEPVTVTWNSGALSSGVHTFPAVLQSGLYTLTSSVTVPSST |
| | | WPSQTVTCNVAHPASSTKVDKKIVPRNCGGDCKPCICTGS |
| | | EVSSVFIFPPKPKDVLTITLTPKVTCVVVDISQDDPEVHFSW |
| | | FVDDVEVHTAQTRPPEEQFNSTFRSVSELPILHQDWLNGR |
| | | TFRCKVTSAAFPSPIEKTISKPEGRTQVPHVYTMSPTKEEM |
| | | TQNEVSITCMVKGFYPPDIYVEWQMNGQPQENYKNTPPT |
| | | MDTDGSYFLYSKLNVKKEKWQQGNTFTCSVLHEGLHNH |
| | | HTEKSLSHSPG |
| 12 | 2.3D11 heavy | QVQLQESGPGLVKPSGTLSLTCAVSGVSIRSINWWNWVR |
| | chain | QPPGKGLEWIGEIYHSGSTNYNPSLKSRVTISVDKSKNQFS |
| | | LKLNSVTAADTAVYYCARDGGIAVTDYYYYGLDVWGQG |
| | | TTVTVSSASTKGPSVFPLAPSSKSTSGGTAALGCLVKDYFP |
| | | EPVTVSWNSGALTSGVHTFPAVLQSSGLYSLSSVVTVPSSS |
| | | LGTQTYICNVNHKPSNTKVDKKVEPKSCDKTHTCPPCPAP |
| | | ELLGGPSVFLFPPKPKDTLMISRTPEVTCVVVDVSHEDPEV |
| | | KFNWYVDGVEVHNAKTKPREEQYNSTYRVVSVLTVLHQ |
| | | DWLNGKEYKCKVSNKALPAPIEKTISKAKGQPREPQVYTL |
| | | PPSRDELTKNQVSLTCLVKGFYPSDIAVEWESNGQPENNY |
| | | KTTPPVLDSDGSFFLYSKLTVDKSRWQQGNVFSCSVMHE |
| | | ALHNHYTQKSLSLSPGK |
| 13 | 2.3D11 heavy | QVQLQESGPGLVKPSGTLSLTCAVSGVSIRSINWWNWVR |
| | chain | QPPGKGLEWIGEIYHSGSTNYNPSLKSRVTISVDKSKNQFS |
| | | LKLNSVTAADTAVYYCARDGGIAVTDYYYYGLDVWGQG |
| | | TTVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPSCPAPEFL |
| | | GGPSVFLFPPKPKDTLMISRTPEVTCVVVDVSQEDPEVQFN |
| | | WYVDGVEVHNAKTKPREEQFNSTYRVVSVLTVLHQDWL |
| | | NGKEYKCKVSNKGLPSSIEKTISKAKGQPREPQVYTLPPSQ |
| | | EEMTKNQVSLTCLVKGFYPSDIAVEWESNGQPENNYKTTP |
| | | PVLDSDGSFFLYSRLTVDKSRWQEGNVFSCSVMHEALHN |
| | | HYTQKSLSLSLGK |
| 14 | 2.3D11 heavy | QVQLQESGPGLVKPSGTLSLTCAVSGVSIRSINWWNWVR |
| | chain | QPPGKGLEWIGEIYHSGSTNYNPSLKSRVTISVDKSKNQFS |
| | | LKLNSVTAADTAVYYCARDGGIAVTDYYYYGLDVWGQG |
| | | TTVTVSSASTKGPSVFPLAPCSRSTSESTAALGCLVKDYFP |
| | | EPVTVSWNSGALTSGVHTFPAVLQSSGLYSLSSVVTVPSSS |
| | | LGTKTYTCNVDHKPSNTKVDKRVESKYGPPCPPCPAPEFE |
| | | GGPSVFLFPPKPKDTLMISRTPEVTCWVDVSQEDPEVQFN |
| | | WYVDGVEVHNAKTKPREEQFNSTYRWSVLTVLHQDWLN |
| | | GKEYKCKVSNKGLPSSIEKTISKAKGQPREPQVYTLPPSQE |
| | | EMTKNQVSLTCLVKGFYPSDIAVEWESNGQPENNYKTTPP |
| | | VLDSDGSFFLYSRLTVDKSRWQEGNVFSCSVMHEALHNH |
| | | YTQKSLSLSLGK |
| 15 | 2.3D11 light | EIVLTQSPATLSLSPGERATLSCRASESVSSNLAWYQQKPG |
| | chain | QAPRLLIYGAFNRATGIPARFSGSGSGTDFTLTISSLEPEDF |
| | | AVYYCQQRSDWFTFGGGTKVEIKRADAAPTVSIFPPSTEQ |
| | | LATGGASVVCLMNNFYPRDISVKWKIDGTERRDGVLDSV |
| | | TDQDSKDSTYSMSSTLSLTKADYESHNLYTCEVVHKTSSS |
| | | PVVKSFNRNEC |
| 16 | 2.3D11 light | EIVLTQSPATLSLSPGERATLSCRASESVSSNLAWYQQKPG |
| | chain | QAPRLLIYGAFNRATGIPARFSGSGSGTDFTLTISSLEPEDF |
| | | AVYYCQQRSDWFTFGGGTKVEIKTVAAPSVFIFPPSDEQL |
| | | KSGTASVVCLLNNFYPREAKVQWKVDNALQSGNSQESVT |
| | | EQDSKDSTYSLSSTLTLSKADYEKHKVYACEVTHQGLSSP |
| | | VTKSFNRGEC |
| 17 | Human kappa | TVAAPSVFIFPPSDEQLKSGTASVVCLLNNFYPREAKVQW |
| | constant region | KVDNALQSGNSQESVTEQDSKDSTYSLSSTLTLSKADYEK |
| | | HKVYACEVTHQGLSSPVTKSFNRGEC |
| 18 | Human IgG1 | ASTKGPSVFPLAPSSKSTSGGTAALGCLVKDYFPEPVTVS |
| | | WNSGALTSGVHTFPAVLQSSGLYSLSSVVTVPSSSLGTQT |
| | | YICNVNHKPSNTKVDKKVEPKSCDKTHTCPPCPAPELLGG |
| | | PSVFLFPPKPKDTLMISRTPEVTCVVVDVSHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPSRD |
| | | ELTKNQVSLTCLVKGFYPSDIAVEWESNGQPENNYKTTPP |
| | | VLDSDGSFFLYSKLTVDKSRWQQGNVFSCSVMHEALHNH |
| | | YTQKSLSLSPGK |
| 19 | Human IgG2 | ASTKGPSVFPLAPCSRSTSESTAALGCLVKDYFPEPVTVSW |
| | | NSGALTSGVHTFPAVLQSSGLYSLSSVVTVPSSNFGTQTYT |
| | | CNVDHKPSNTKVDKTVERKCCVECPPCPAPPVAGPSVF |
| | | LFPPKPKDTLMISRTPEVTCVVVDVSHEDPEVQFNWYVDG |
| | | VEVHNAKTKPREEQFNSTFRVVSVLTVVHQDWLNGKEYK |
| | | CKVSNKGLPAPIEKTISKTKGQPREPQVYTLPPSREEMTKN |
| | | QVSLTCLVKGFYPSDISVEWESNGQPENNYKTTPPMLDSD |
| | | GSFFLYSKLTVDKSRWQQGNVFSCSVMHEALHNHYTQKS |
| | | LSLSPGK |
| 20 | Human IgG3 | ASTKGPSVFPLAPCSRSTSGGTAALGCLVKDYFPEPVTVS |
| | | WNSGALTSGVHTFPAVLQSSGLYSLSSVVTVPSSSLGTQT |
| | | YTCNVNHKPSNTKVDKRVELKTPLGDTTHTCPRCPEPKSC |
| | | DTPPPCPRCPEPKSCDTPPPCPRCPEPKSCDTPPPCPRCPAP |
| | | ELLGGPSVFLFPPKPKDTLMISRTPEVTCVVVDVSHEDPEV |
| | | QFKWYVDGVEVHNAKTKPREEQYNSTFRVVSVLTVLH |
| | | QDWLNGKEYKCKVSNKALPAPIEKTISKTKGQPREPQVYT |
| | | LPPSREEMTKNQVSLTCLVKGFYPSDIAVEWESSGQPENN |
| | | YNTTPPMLDSDGSFFLYSKLTVDKSRWQQGNIFSCSVMHE |
| | | ALHNRFTQKSLSLSPGK |
| 21 | Human IgG4 | ASTKGPSVFPLAPCSRSTSESTAALGCLVKDYFPEPVTVSW |
| | | NSGALTSGVHTFPAVLQSSGLYSLSSVVTVPSSSLGTKTYT |
| | | CNVDHKPSNTKVDKRVESKYGPPCPSCPAPEFLGGPSVFL |
| | | FPPKPKDTLMISRTPEVTCVVVDVSQEDPEVQFNWYVDG |
| | | VEVHNAKTKPREEQFNSTYRVVSVLTVLHQDWLNGKEY |
| | | KCKVSNKGLPSSIEKTISKAKGQPREPQVYTLPPSQEEMTK |
| | | NQVSLTCLVKGFYPSDIAVEWESNGQPENNYKTTPPVLDS |
| | | DGSFFLYSRLTVDKSRWQEGNVFSCSVMHEALHNHYTQK |
| | | SLSLSLGK |
| 22 | FLAG tag | DYKDDDDK |
| 23 | Polyhistidine | HHHHHH |
| | tag | |
| 24 | Hemagglutinin | YPYDVPDYA |
| | tag | |
| 25 | 2.3D11 heavy | AAGCTTACCGCCACCATGGGTTGGTCCTGCATCATCCTG |
| | chain with | TTCCTGGTGGCCACGGCCACCGGCGTGCACTCCCAAGT |
| | human IgG1 | CCAACTCCAGGAGTCCGGCCCCGGGCTGGTCAAGCCGT |
| | | CCGGCACACTGTCCCTGACGTGCGCCGTCTCCGGGGTCT |
| | | CTATCCGGAGCATCAACTGGTGGAATTGGGTGCGGCAG |
| | | CCGCCCGGTAAGGGCCTCGAGTGGATTGGCGAGATCTA |
| | | CCACTCAGGCAGCACCAACTACAACCCCTCCCTCAAGT |
| | | CGCGCGTCACGATCTCGGTCGACAAGTCCAAGAACCAG |
| | | TTCTCGCTCAAGCTCAACAGCGTGACCGCGGCGGACAC |
| | | CGCCGTGTACTACTGTGCCCGGGACGGCGGCATCGCAG |
| | | TCACTGACTACTACTATTACGGCCTCGACGTGTGGGGCC |
| | | AGGGGACGACGGTCACGGTGAGCTCCGCCTCCACCAAA |
| | | GGCCCCAGCGTCTTCCCCCTCGCGCCGTCCTCCAAGTCC |
| | | ACCTCGGGTGGCACCGCCGCCCTGGGCTGCCTGGTCAA |
| | | GGACTACTTCCCGGAGCCTGTGACCGTGTCCTGGAACTC |
| | | GGGCGCGCTCACGAGCGGCGTACACACCTTCCCGGCGG |
| | | TGCTCCAGTCCTCCGGGCTGTACTCGCTCTCGTCGGTCG |
| | | TCACGGTGCCGTCCTCCTCCCTGGGCACCCAGACCTACA |
| | | TCTGCAACGTGAACCACAAGCCGTCCAACACCAAGGTG |
| | | GATAAGAAGGTCGAGCCCAAGTCGTGCGACAAGACGC |
| | | ACACGTGCCCGCCGTGCCCGGCCCCGGAGCTGCTGGGC |
| | | GGCCCCTCGGTCTTCCTGTTCCCCCCGAAGCCCAAGGAT |
| | | ACGCTGATGATCTCCCGCACCCCGGAGGTCACCTGCGT |
| | | GGTGGTGGACGTCTCCCACGAGGACCCGGAGGTGAAAT |
| | | TCAACTGGTACGTCGACGGAGTGGAGGTCCACAACGCC |
| | | AAGACCAAGCCCCGGGAGGAGCAGTACAACTCCACGTA |
| | | CCGCGTCGTCTCCGTCCTGACCGTCCTCCACCAGGACTG |
| | | GCTGAACGGCAAGGAGTACAAGTGTAAGGTCTCCAACA |
| | | AGGCGCTGCCCGCCCCCATCGAGAAGACCATCTCCAAG |
| | | GCAAAGGGTCAGCCGCGGGAGCCGCAGGTCTATACCCT |
| | | CCCCCCGTCCCGCGACGAGCTGACGAAAAACCAGGTCT |
| | | CCCTGACCTGCCTGGTGAAGGGTTTCTACCCCTCCGACA |
| | | TCGCGGTCGAGTGGGAGTCGAACGGCCAGCCGGAGAAC |
| | | AACTACAAGACCACCCCCCCCGTGCTCGACAGTGACGG |
| | | CTCGTTCTTCCTGTACTCGAAGCTGACCGTCGACAAGTC |
| | | GCGCTGGCAGCAGGGCAACGTCTTCTCGTGCTCCGTTAT |
| | | GCACGAGGCCCTGCACAACCACTACACGCAGAAGAGTC |
| | | TTTCGCTGTCCCCGGGGAAGTGATAATCTAGAGTCGGG |
| | | GCGGCCGGCC |
| 26 | 2.3D11 heavy | AAGCTTACCGCCACCATGGGGTGGTCGTGCATCATCCTC |
| | chain with | TTCCTGGTCGCCACCGCGACCGGCGTGCATTCGCAGGTC |
| | human IgG4 | CAGCTCCAGGAGAGCGGCCCGGGCCTGGTGAAGCCCTC |
| | | CGGCACGCTCTCTCTGACGTGCGCCGTCTCGGGAGTGA |
| | | GTATCCGCTCGATCAACTGGTGGAACTGGGTGCGGCAG |
| | | CCGCCGGGCAAGGGCCTGGAATGGATCGGGGAGATCTA |
| | | CCACTCCGGGTCGACCAACTACAACCCGAGCCTGAAGT |
| | | CCCGGGTCACGATCAGCGTGGACAAGTCCAAGAACCAG |
| | | TTCTCCCTGAAGCTGAACAGTGTAACGGCGGCGGACAC |
| | | GGCGGTCTACTACTGTGCGCGCGACGGCGGCATCGCCG |
| | | TGACCGATTACTACTACTACGGCCTCGACGTATGGGGC |
| | | CAGGGCACCACCGTCACGGTGTCGAGCGCATCGACGAA |
| | | GGGCCCCTCCGTGTTCCCCCTAGCCCCGTGCTCCCGCAG |
| | | CACCTCTGAGTCCACGGCGGCCTTGGGCTGCCTCGTGA |
| | | AGGACTACTTCCCGGAGCCGGTCACTGTGTCGTGGAAC |
| | | TCCGGCGCGCTGACCAGCGGGGTCCACACCTTCCCCGC |
| | | CGTCCTGCAGTCGTCGGGCCTGTACTCCCTGAGCTCGGT |
| | | GGTGACCGTCCCCTCCAGCTCCCTCGGCACTAAGACCTA |
| | | TACCTGCAACGTCGACCACAAGCCGTCCAACACCAAGG |
| | | TGGACAAGCGAGTGGAATCGAAGTACGGCCCGCCCTGC |
| | | CCCTCCTGCCCCGCCCCCGAGTTCCTGGGGGGCCCGAG |
| | | CGTCTTCCTGTTCCCGCCGAAGCCGAAGGACACGCTGA |
| | | TGATCAGCCGGACGCCGGAAGTGACGTGCGTCGTCGTG |
| | | GACGTGTCCCAGGAAGACCCTGAGGTGCAGTTCAACTG |
| | | GTACGTGGACGGCGTGGAGGTGCACAACGCGAAAACC |
| | | AAGCCGCGCGAGGAGCAGTTCAACAGCACCTACCGCGT |
| | | CGTGAGCGTCCTGACGGTGCTGCACCAGGACTGGCTCA |
| | | ACGGCAAGGAGTACAAGTGCAAGGTATCCAACAAGGG |
| | | ACTGCCGTCGTCCATCGAGAAGACCATCTCCAAGGCCA |
| | | AGGGCCAGCCCCGGGAGCCCCAAGTCTACACCCTCCCC |
| | | CCGTCGCAGGAGGAGATGACGAAGAACCAGGTCTCCCT |
| | | GACCTGTCTCGTCAAGGGCTTCTACCCCTCCGACATCGC |
| | | CGTCGAGTGGGAGTCCAACGGGCAGCCCGAGAACAACT |
| | | ACAAGACCACCCCGCCAGTCCTGGACAGTGACGGGTCG |
| | | TTCTTCCTGTACTCCCGACTCACTGTGGACAAGAGCCGC |
| | | TGGCAAGAGGGGAACGTCTTCTCCTGCTCAGTGATGCA |
| | | CGAGGCCCTCCACAACCACTACACCCAAAAGTCGCTGT |
| | | CCCTGTCCCTCGGGAAATGATAATCTAGAGTCGGGGCG |
| | | GCCGGCC |
| 27 | 2.3D11 heavy | AAGCTTACCGCCACCATGGGGTGGTCGTGCATCATCCTC |
| | chain with | TTCCTGGTCGCCACCGCGACCGGCGTGCATTCGCAGGTC |
| | human IgG4 | CAGCTCCAGGAGAGCGGCCCGGGCCTGGTGAAGCCCTC |
| | Ser228Pro and | CGGCACGCTCTCTCTGACGTGCGCCGTCTCGGGAGTGA |
| | Leu235Glu | GTATCCGCTCGATCAACTGGTGGAACTGGGTGCGGCAG |
| | | CCGCCGGGCAAGGGCCTGGAATGGATCGGGGAGATCTA |
| | | CCACTCCGGGTCGACCAACTACAACCCGAGCCTGAAGT |
| | | CCCGGGTCACGATCAGCGTGGACAAGTCCAAGAACCAG |
| | | TTCTCCCTGAAGCTGAACAGTGTAACGGCGGCGGACAC |
| | | GGCGGTCTACTACTGTGCGCGCGACGGCGGCATCGCCG |
| | | TGACCGATTACTACTACTACGGCCTCGACGTATGGGGC |
| | | CAGGGCACCACCGTCACGGTGTCGAGCGCATCGACGAA |
| | | GGGCCCCTCCGTGTTCCCCCTAGCCCCGTGCTCCCGCAG |
| | | CACCTCTGAGTCCACGGCGGCCTTGGGCTGCCTCGTGA |
| | | AGGACTACTTCCCGGAGCCGGTCACTGTGTCGTGGAAC |
| | | TCCGGCGCGCTGACCAGCGGGGTCCACACCTTCCCCGC |
| | | CGTCCTGCAGTCGTCGGGCCTGTACTCCCTGAGCTCGGT |
| | | GGTGACCGTCCCCTCCAGCTCCCTCGGCACTAAGACCTA |
| | | TACCTGCAACGTCGACCACAAGCCGTCCAACACCAAGG |
| | | TGGACAAGCGAGTGGAATCGAAGTACGGCCCGCCCTGC |
| | | CCCCCCTGCCCCGCCCCCGAGTTCGAGGGCGGGCCGAG |
| | | CGTCTTCCTGTTCCCGCCGAAGCCGAAGGACACGCTGA |
| | | TGATCAGCCGGACGCCGGAAGTGACGTGCGTCGTCGTG |
| | | GACGTGTCCCAGGAAGACCCTGAGGTGCAGTTCAACTG |
| | | GTACGTGGACGGCGTGGAGGTGCACAACGCGAAAACC |
| | | AAGCCGCGCGAGGAGCAGTTCAACAGCACCTACCGCGT |
| | | CGTGAGCGTCCTGACGGTGCTGCACCAGGACTGGCTCA |
| | | ACGGCAAGGAGTACAAGTGCAAGGTATCCAACAAGGG |
| | | ACTGCCGTCGTCCATCGAGAAGACCATCTCCAAGGCCA |
| | | AGGGCCAGCCCCGGGAGCCCCAAGTCTACACCCTCCCC |
| | | CCGTCGCAGGAGGAGATGACGAAGAACCAGGTCTCCCT |
| | | GACCTGTCTCGTCAAGGGCTTCTACCCCTCCGACATCGC |
| | | CGTCGAGTGGGAGTCCAACGGGCAGCCCGAGAACAACT |
| | | ACAAGACCACCCCGCCAGTCCTGGACAGTGACGGGTCG |
| | | TTCTTCCTGTACTCCCGACTCACTGTGGACAAGAGCCGC |
| | | TGGCAAGAGGGGAACGTCTTCTCCTGCTCAGTGATGCA |
| | | CGAGGCCCTCCACAACCACTACACCCAAAAGTCGCTGT |
| | | CCCTGTCCCTCGGGAAATGATAATCTAGAGTCGGGGCG |
| | | GCCGGCC |
| 28 | 2.3D11 light | AAGCTTACCGCCACCATGGGGTGGTCGTGCATCATCCTC |
| | human kappa | CGTCCTGACCCAGTCCCCCGCCACCCTCTCCCTGTCGCC |
| | | GGGCGAGCGGGCCACGCTGTCGTGCCGGGCGTCCGAGT |
| | | CGGTCTCGTCGAACCTCGCCTGGTATCAGCAGAAGCCC |
| | | GGCCAGGCGCCGCGCCTCCTCATCTACGGCGCCTTCAAT |
| | | CGCGCCACGGGCATCCCCGCCCGGTTCTCCGGCTCCGG |
| | | ATCGGGGACCGACTTCACCCTCACCATCTCCTCGCTGGA |
| | | GCCGGAGGACTTCGCCGTCTACTACTGCCAGCAACGGT |
| | | CGGACTGGTTCACCTTCGGAGGCGGCACCAAGGTCGAG |
| | | ATCAAGACGGTGGCCGCGCCGAGCGTCTTCATCTTCCC |
| | | GCCTTCCGACGAGCAGCTCAAGTCCGGGACCGCCTCCG |
| | | TAGTATGCCTCCTCAATAACTTCTACCCCCGGGAGGCGA |
| | | AGGTCCAGTGGAAGGTCGACAACGCCCTCCAATCGGGC |
| | | AACTCCCAGGAGTCGGTGACCGAGCAGGATTCCAAGGA |
| | | CTCGACCTACAGTCTAAGCTCCACCCTCACACTGTCGAA |
| | | GGCGGACTACGAGAAGCACAAGGTGTACGCCTGCGAG |
| | | GTCACCCACCAGGGCCTGAGCAGCCCGGTCACCAAGTC |
| | | CTTCAACCGGGGCGAGTGCTGATAATCTAGAGTCGGGG |
| | | CGGCCGGCC |

1. A method for treating or delaying progression of a cancer or reducing or inhibiting tumor growth in a subject in need thereof, the method comprising administering to the subject an effective amount of a monoclonal antibody that specifically binds human CD47, or an antigen binding fragment thereof, and an effective amount of an agent that induces apoptosis.

2. The method of claim 1, wherein the cell death-inducing agent is an agent that induces apoptosis selected from the group consisting of: an inhibitor of BCL-2, an inhibitor of MCL-1, an inhibitor of BCL-XL, an inhibitor of MDM2, and a combination thereof.

3. (canceled)

4. The method of claim 2, wherein the inhibitor of BCL-2 is selected from Venetoclax™, Navitoclax, or obatoclax.

5. The method of claim 4, wherein the inhibitor of BCL-2 is Venetoclax™.

6.-29. (canceled)

30. The method of claim 1, further comprising administering an effective amount of a hypomethylating agent or an effective amount of a chemotherapeutic agent.

31.-37. (canceled)

38. The method of claim 1, wherein the monoclonal antibody that specifically binds human CD47, or antigen binding fragment thereof, comprises:
a heavy chain complementarity determining region 1 (HC CDR1) comprising the amino acid sequence set forth in SEQ ID NO: 5;
a heavy chain complementarity determining region 2 (HC CDR2) comprising the amino acid sequence set forth in SEQ ID NO: 6;
a heavy chain complementarity determining region 3 (HC CDR3) comprising the amino acid sequence set forth in SEQ ID NO: 7;
a light chain complementarity determining region 1 (LC CDR1) comprising the amino acid sequence set forth in SEQ ID NO: 8;
a light chain complementarity determining region 2 (LC CDR2) comprising the amino acid sequence set forth in SEQ ID NO: 9; and
a light chain complementarity determining region 3 (LC CDR3) comprising the amino acid sequence set forth in SEQ ID NO: 10.

39. The method of claim 1, wherein the monoclonal antibody that specifically binds to human CD47, or antigen binding fragment thereof, comprises a heavy chain variable region (VH) comprising the amino acid sequence set forth in SEQ ID NO: 3 and a light chain variable region (V_{L}) comprising the amino acid sequence set forth in SEQ ID NO: 4.

40.-144. (canceled)

145. A kit comprising a monoclonal antibody that specifically binds human CD47, or an antigen binding fragment thereof, and a package insert comprising instructions for administration of the monoclonal antibody or antigen binding fragment thereof in combination with a second composition comprising an agent that induces apoptosis, for treating or delaying progression of cancer or reducing or inhibiting tumor growth in a subject in need thereof.

146. (canceled)

147. A method for treating or delaying progression of a cancer or reducing or inhibiting tumor growth in a subject in need thereof, the method comprising administering to the subject an effective amount of a monoclonal antibody that specifically binds human CD47, or antigen binding fragment thereof, and an effective amount of an agent that induces immunogenic cell death (ICD).

148. The method of claim 147, wherein the agent that induces ICD is selected from an anthracycline, a proteasome inhibitor, and a platinum derivative.

149. The method of claim 148, wherein the agent that induces ICD is an anthracycline, and wherein the anthracycline is selected from doxorubicin, daunorubicin, epirubicin, idarubicin, and valrubicin.

150. The method of claim 148, wherein the agent that induces ICD is a proteasome inhibitor, and wherein the proteasome inhibitor is selected from bortezomib, carfilzomib, and ixazomib.

151. The method of claim 148, wherein the agent that induces ICD is a platinum derivative, and wherein the platinum derivative is selected from oxaliplatin, carboplatin, and cisplatin.

152. A kit comprising a monoclonal antibody that specifically binds human CD47, or an antigen binding fragment thereof, and a package insert comprising instructions for administration of the monoclonal antibody or antigen binding fragment thereof in combination with a second composition comprising an agent that induces immunogenic cell death (ICD), for treating or delaying progression of cancer or reducing or inhibiting tumor growth in a subject in need thereof.

153. A method for treating or delaying progression of a cancer or reducing or inhibiting tumor growth in a subject in need thereof, the method comprising administering to the subject an effective amount of a monoclonal antibody that specifically binds human CD47, or antigen binding fragment thereof, and an effective amount of an agent that inhibits a DNA damage response pathway.

154. The method of claim 153, wherein the agent that inhibits a DNA damage response pathway is an inhibitor of poly ADP ribose polymerase (PARP), and wherein the inhibitor of PARP is selected from the group consisting of Olaparib, Niraparib, and Rucaparib.

155. The method of claim 153, wherein the agent that inhibits a DNA damage response pathway is temozolomide.

156. A kit comprising a monoclonal antibody that specifically binds human CD47, or an antigen binding fragment thereof, and a package insert comprising instructions for administration of the monoclonal antibody or antigen binding fragment thereof in combination with a second composition comprising agent that inhibits a DNA damage response pathway, for treating or delaying progression of cancer or reducing or inhibiting tumor growth in a subject in need thereof.


SEQUENCE LISTING (28 sequences)

SEQ ID NO: 1 - protein, 323 aa, Homo sapiens
  misc_feature (1)..(323): Human CD47 protein
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 5345 aa, Homo sapiens
  misc_feature (1)..(5345): Human CD47 mRNA
GGGGAGCAGGCGGGGGAGCGGGCGGGAAGCAGTGGGAGCGCGCGTGCGCGCGGCCGTGCAGCCTGGGCAGTGGGTCCTGCCTGTGACGCGCGGCGGCGGTCGGTCCTGCCTGTAACGGCGGCGGCGGCTGCTGCTCCAGACACCTGCGGCGGCGGCGGCGACCCCGCGGCGGGCGCGGAGATGTGGCCCCTGGTAGCGGCGCTGTTGCTGGGCTCGGCGTGCTGCGGATCAGCTCAGCTACTATTTAATAAAACAAAATCTGTAGAATTCACGTTTTGTAATGACACTGTCGTCATTCCATGCTTTGTTACTAATATGGAGGCACAAAACACTACTGAAGTATACGTAAAGTGGAAATTTAAAGGAAGAGATATTTACACCTTTGATGGAGCTCTAAACAAGTCCACTGTCCCCACTGACTTTAGTAGTGCAAAAATTGAAGTCTCACAATTACTAAAAGGAGATGCCTCTTTGAAGATGGATAAGAGTGATGCTGTCTCACACACAGGAAACTACACTTGTGAAGTAACAGAATTAACCAGAGAAGGTGAAACGATCATCGAGCTAAAATATCGTGTTGTTTCATGGTTTTCTCCAAATGAAAATATTCTTATTGTTATTTTCCCAATTTTTGCTATACTCCTGTTCTGGGGACAGTTTGGTATTAAAACACTTAAATATAGATCCGGTGGTATGGATGAGAAAACAATTGCTTTACTTGTTGCTGGACTAGTGATCACTGTCATTGTCATTGTTGGAGCCATTCTTTTCGTCCCAGGTGAATATTCATTAAAGAATGCTACTGGCCTTGGTTTAATTGTGACTTCTACAGGGATATTAATATTACTTCACTACTATGTGTTTAGTACAGCGATTGGATTAACCTCCTTCGTCATTGCCATATTGGTTATTCAGGTGATAGCCTATATCCTCGCTGTGGTTGGACTGAGTCTCTGTATTGCGGCGTGTATACCAATGCATGGCCCTCTTCTGATTTCAGGTTTGAGTATCTTAGCTCTAGCACAATTACTTGGACTAGTTTATATGAAATTTGTGGCTTCCAATCAGAAGACTATACAACCTCCTAGGAAGCTGTAGAGGAACCCCTTAATGCATTCAAAGAATCAAAAGGAATGATGAATGATGAATAACTGAAGTGAAGTGATGGACTCCGATTTGGAGAGTAGTAAGACGTGAAAGGAATACACTTGTGTTTAAGCACCATGGCCTTGATGATTCACTGTTGGGGAGAAGAAACAAGAAAAGTAACTGGTTGTCACCTATGAGACCCTTACGTGATTGTTAGTTAAGTTTTTATTCAAAGCAGCTGTAATTTAGTTAATAAAATAATTATGATCTATGTTGTTTGCCCAATTGAGATCCAGTTTTTTGTTGTTATTTTTAATCAATTAGGGGCAATAGTAGAATGGACAATTTCCAAGAATGATGCCTTTCAGGTCCTAGGGCCTCTGGCCTCTAGGTAACCAGTTTAAATTGGTTCAGGGTGATAACTACTTAGCACTGCCCTGGTGATTACCCAGAGATATCTATGAAAACCAGTGGCTTCCATCAAACCTTTGCCAACTCAGGTTCACAGCAGCTTTGGGCAGTTATGGCAGTATGGCATTAGCTGAGAGGTGTCTGCCACTTCTGGGTCAATGGAATAATAAATTAAGTACAGGCAGGAATTTGGTTGGGAGCATCTTGTATGATCTCCGTATGATGTGATATTGATGGAGATAGTGGTCCTCATTCTTGGGGGTTGCCATTCCCACATTCCCCCTTCAACAAACAGTGTAACAGGTCCTTCCCAGATTTAGGGTACTTTTATTGATGGATATGTTTTCCTTTTATTCACATAACCCCTTGAAACCCTGTCTTGTCCTCCTGTTACTTGCTTCTGCTGTACAAGATGTAGCACCTTTTCTCCTCTTTGAACATGGTCTAGTGACACGGTAGCACCAGTTGCAGGAAGGAGCCAGACTTGTTCTCAGAGCACTGTGTTCACACTTTTCAGCAAAAATAGCTATGGTTGTAACATATGTATTCCCTTCCTCTGATTTGAAGGCAAAAATCTACAGTGTTTCTTCACTTCTTTTCTGATCTGGGGCATGAAAAAAGCAAGATTGAAATTTGAACTATGAGTCTCCTGCATGGCAACAAAATGTGTGTCACCATCAGGCCAACAGGCCAGCCCTTGAATGGGGATTTATTACTGTTGTATCTATGTTGCATGATAAACATTCATCACCTTCCTCCTGTAGTCCTGCCTCGTACTCCCCTTCCCCTATGATTGAAAAGTAAACAAAACCCACATTTCCTATCCTGGTTAGAAGAAAATTAATGTTCTGACAGTTGTGATCGCCTGGAGTACTTTTAGACTTTTAGCATTCGTTTTTTACCTGTTTGTGGATGTGTGTTTGTATGTGCATACGTATGAGATAGGCACATGCATCTTCTGTATGGACAAAGGTGGGGTACCTACAGGAGAGCAAAGGTTAATTTTGTGCTTTTAGTAAAAACATTTAAATACAAAGTTCTTTATTGGGTGGAATTATATTTGATGCAAATATTTGATCACTTAAAACTTTTAAAACTTCTAGGTAATTTGCCACGCTTTTTGACTGCTCACCAATACCCTGTAAAAATACGTAATTCTTCCTGTTTGTGTAATAAGATATTCATATTTGTAGTTGCATTAATAATAGTTATTTCTTAGTCCATCAGATGTTCCCGTGTGCCTCTTTTATGCCAAATTGATTGTCATATTTCATGTTGGGACCAAGTAGTTTGCCCATGGCAAACCTAAATTTATGACCTGCTGAGGCCTCTCAGAAAACTGAGCATACTAGCAAGACAGCTCTTCTTGAAAAAAAAAATATGTATACACAAATATATACGTATATCTATATATACGTATGTATATACACACATGTATATTCTTCCTTGATTGTGTAGCTGTCCAAAATAATAACATATATAGAGGGAGCTGTATTCCTTTATACAAATCTGATGGCTCCTGCAGCACTTTTTCCTTCTGAAAATATTTACATTTTGCTAACCTAGTTTGTTACTTTAAAAATCAGTTTTGATGAAAGGAGGGAAAAGCAGATGGACTTGAAAAAGATCCAAGCTCCTATTAGAAAAGGTATGAAAATCTTTATAGTAAAATTTTTTATAAACTAAAGTTGTACCTTTTAATATGTAGTAAACTCTCATTTATTTGGGGTTCGCTCTTGGATCTCATCCATCCATTGTGTTCTCTTTAATGCTGCCTGCCTTTTGAGGCATTCACTGCCCTAGACAATGCCACCAGAGATAGTGGGGGAAATGCCAGATGAAACCAACTCTTGCTCTCACTAGTTGTCAGCTTCTCTGGATAAGTGACCACAGAAGCAGGAGTCCTCCTGCTTGGGCATCATTGGGCCAGTTCCTTCTCTTTAAATCAGATTTGTAATGGCTCCCAAATTCCATCACATCACATTTAAATTGCAGACAGTGTTTTGCACATCATGTATCTGTTTTGTCCCATAATATGCTTTTTACTCCCTGATCCCAGTTTCTGCTGTTGACTCTTCCATTCAGTTTTATTTATTGTGTGTTCTCACAGTGACACCATTTGTCCTTTTCTGCAACAACCTTTCCAGCTACTTTTGCCAAATTCTATTTGTCTTCTCCTTCAAAACATTCTCCTTTGCAGTTCCTCTTCATCTGTGTAGCTGCTCTTTTGTCTCTTAACTTACCATTCCTATAGTACTTTATGCATCTCTGCTTAGTTCTATTAGTTTTTTGGCCTTGCTCTTCTCCTTGATTTTAAAATTCCTTCTATAGCTAGAGCTTTTCTTTCTTTCATTCTCTCTTCCTGCAGTGTTTTGCATACATCAGAAGCTAGGTACATAAGTTAAATGATTGAGAGTTGGCTGTATTTAGATTTATCACTTTTTAATAGGGTGAGCTTGAGAGTTTTCTTTCTTTCTGTTTTTTTTTTTTGTTTTTTTTTTTTTTTTTTTTTTTTTTTTTTTTGACTAATTTCACATGCTCTAAAAACCTTCAAAGGTGATTATTTTTCTCCTGGAAACTCCAGGTCCATTCTGTTTAAATCCCTAAGAATGTCAGAATTAAAATAACAGGGCTATCCCGTAATTGGAAATATTTCTTTTTTCAGGATGCTATAGTCAATTTAGTAAGTGACCACCAAATTGTTATTTGCACTAACAAAGCTCAAAACACGATAAGTTTACTCCTCCATCTCAGTAATAAAAATTAAGCTGTAATCAACCTTCTAGGTTTCTCTTGTCTTAAAATGGGTATTCAAAAATGGGGATCTGTGGTGTATGTATGGAAACACATACTCCTTAATTTACCTGTTGTTGGAAACTGGAGAAATGATTGTCGGGCAACCGTTTATTTTTTATTGTATTTTATTTGGTTGAGGGATTTTTTTATAAACAGTTTTACTTGTGTCATATTTTAAAATTACTAACTGCCATCACCTGCTGGGGTCCTTTGTTAGGTCATTTTCAGTGACTAATAGGGATAATCCAGGTAACTTTGAAGAGATGAGCAGTGAGTGACCAGGCAGTTTTTCTGCCTTTAGCTTTGACAGTTCTTAATTAAGATCATTGAAGACCAGCTTTCTCATAAATTTCTCTTTTTGAAAAAAAGAAAGCATTTGTACTAAGCTCCTCTGTAAGACAACATCTTAAATCTTAAAAGTGTTGTTATCATGACTGGTGAGAGAAGAAAACATTTTGTTTTTATTAAATGGAGCATTATTTACAAAAAGCCATTGTTGAGAATTAGATCCCACATCGTATAAATATCTATTAACCATTCTAAATAAAGAGAACTCCAGTGTTGCTATGTGCAAGATCCTCTCTTGGAGCTTTTTTGCATAGCAATTAAAGGTGTGCTATTTGTCAGTAGCCATTTTTTTGCAGTGATTTGAAGACCAAAGTTGTTTTACAGCTGTGTTACCGTTAAAGGTTTTTTTTTTTATATGTATTAAATCAATTTATCACTGTTTAAAGCTTTGAATATCTGCAATCTTTGCCAAGGTACTTTTTTATTTAAAAAAAAACATAACTTTGTAAATATTACCCTGTAATATTATATATACTTAATAAAACATTTTAAGCTATTTTGTTGGGCTATTTCTATTGCTGCTACAGCAGACCACAAGCACATTTCTGAAAAATTTAATTTATTAATGTATTTTTAAGTTGCTTATATTCTAGGTAACAATGTAAAGAATGATTTAAAATATTAATTATGAATTTTTTGAGTATAATACCCAATAAGCTTTTAATTAGAGCAGAGTTTTAATTAAAAGTTTTAAATCAGTC

SEQ ID NO: 3 - protein, 125 aa, Artificial Sequence
  Synthetic 2.3D11 VH
QVQLQESGPGLVKPSGTLSLTCAVSGVSIRSINWWNWVRQPPGKGLEWIGEIYHSGSTNYNPSLKSRVTISVDKSKNQFSLKLNSVTAADTAVYYCARDGGIAVTDYYYYGLDVWGQGTTVTVSS

SEQ ID NO: 4 - protein, 106 aa, Artificial Sequence
  Synthetic 2.3D11 VL
EIVLTQSPATLSLSPGERATLSCRASESVSSNLAWYQQKPGQAPRLLIYGAFNRATGIPARFSGSGSGTDFTLTISSLEPEDFAVYYCQQRSDWFTFGGGTKVEIK

SEQ ID NO: 5 - protein, 6 aa, Artificial Sequence
  Synthetic 2.3D11 HCDR1
SINWWN

SEQ ID NO: 6 - protein, 16 aa, Artificial Sequence
  Synthetic 2.3D11 HCDR2
EIYHSGSTNYNPSLKS

SEQ ID NO: 7 - protein, 16 aa, Artificial Sequence
  Synthetic 2.3D11 HCDR3
DGGIAVTDYYYYGLDV

SEQ ID NO: 8 - protein, 11 aa, Artificial Sequence
  Synthetic 2.3D11 LCDR1
RASESVSSNLA

SEQ ID NO: 9 - protein, 7 aa, Artificial Sequence
  Synthetic 2.3D11 LCDR2
GAFNRAT

SEQ ID NO: 10 - protein, 8 aa, Artificial Sequence
  Synthetic 2.3D11 LCDR3
QQRSDWFT

SEQ ID NO: 11 - protein, 450 aa, Artificial Sequence
  Synthetic 2.3D11 heavy chain
QVQLQESGPGLVKPSGTLSLTCAVSGVSIRSINWWNWVRQPPGKGLEWIGEIYHSGSTNYNPSLKSRVTISVDKSKNQFSLKLNSVTAADTAVYYCARDGGIAVTDYYYYGLDVWGQGTTVTVSSAETTAPSVYPLAPGTALKSNSMVTLGCLVKGYFPEPVTVTWNSGALSSGVHTFPAVLQSGLYTLTSSVTVPSSTWPSQTVTCNVAHPASSTKVDKKIVPRNCGGDCKPCICTGSEVSSVFIFPPKPKDVLTITLTPKVTCVVVDISQDDPEVHFSWFVDDVEVHTAQTRPPEEQFNSTFRSVSELPILHQDWLNGRTFRCKVTSAAFPSPIEKTISKPEGRTQVPHVYTMSPTKEEMTQNEVSITCMVKGFYPPDIYVEWQMNGQPQENYKNTPPTMDTDGSYFLYSKLNVKKEKWQQGNTFTCSVLHEGLHNHHTEKSLSHSPG

SEQ ID NO: 12 - protein, 455 aa, Artificial Sequence
  Synthetic 2.3D11 heavy chain
QVQLQESGPGLVKPSGTLSLTCAVSGVSIRSINWWNWVRQPPGKGLEWIGEIYHSGSTNYNPSLKSRVTISVDKSKNQFSLKLNSVTAADTAVYYCARDGGIAVTDYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 452 aa, Artificial Sequence
  Synthetic 2.3D11 heavy chain
QVQLQESGPGLVKPSGTLSLTCAVSGVSIRSINWWNWVRQPPGKGLEWIGEIYHSGSTNYNPSLKSRVTISVDKSKNQFSLKLNSVTAADTAVYYCARDGGIAVTDYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 450 aa, Artificial Sequence
  Synthetic 2.3D11 heavy chain
QVQLQESGPGLVKPSGTLSLTCAVSGVSIRSINWWNWVRQPPGKGLEWIGEIYHSGSTNYNPSLKSRVTISVDKSKNQFSLKLNSVTAADTAVYYCARDGGIAVTDYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCWVDVSQEDPEVQFNWYVDGVEVHNAKTKPREEQFNSTYRWSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 213 aa, Artificial Sequence
  Synthetic 2.3D11 light chain
EIVLTQSPATLSLSPGERATLSCRASESVSSNLAWYQQKPGQAPRLLIYGAFNRATGIPARFSGSGSGTDFTLTISSLEPEDFAVYYCQQRSDWFTFGGGTKVEIKRADAAPTVSIFPPSTEQLATGGASVVCLMNNFYPRDISVKWKIDGTERRDGVLDSVTDQDSKDSTYSMSSTLSLTKADYESHNLYTCEVVHKTSSSPVVKSFNRNEC

SEQ ID NO: 16 - protein, 212 aa, Artificial Sequence
  Synthetic 2.3D11 light chain
EIVLTQSPATLSLSPGERATLSCRASESVSSNLAWYQQKPGQAPRLLIYGAFNRATGIPARFSGSGSGTDFTLTISSLEPEDFAVYYCQQRSDWFTFGGGTKVEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 106 aa, Homo sapiens
  misc_feature (1)..(106): Human kappa constant region
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 330 aa, Homo sapiens [sequence omitted]

SEQ ID NO: 19 - protein, 326 aa, Homo sapiens
  misc_feature (1)..(326): Human IgG2
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 20 - protein, 377 aa, Homo sapiens
  misc_feature (1)..(377): Human IgG3
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 21 - protein, 327 aa, Homo sapiens
  misc_feature (1)..(327): Human IgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 8 aa, Artificial Sequence
  Synthetic FLAG tag
DYKDDDDK

SEQ ID NO: 23 - protein, 6 aa, Artificial Sequence
  Synthetic Polyhistidine tag
HHHHHH

SEQ ID NO: 24 - protein, 9 aa, Artificial Sequence
  Synthetic Hemagglutinin tag
YPYDVPDYA

SEQ ID NO: 25 - DNA, 1465 bp, Artificial Sequence
  Synthetic 2.3D11 heavy chain with human IgG1
aagcttaccgccaccatgggttggtcctgcatcatcctgttcctggtggccacggccaccggcgtgcactcccaagtccaactccaggagtccggccccgggctggtcaagccgtccggcacactgtccctgacgtgcgccgtctccggggtctctatccggagcatcaactggtggaattgggtgcggcagccgcccggtaagggcctcgagtggattggcgagatctaccactcaggcagcaccaactacaacccctccctcaagtcgcgcgtcacgatctcggtcgacaagtccaagaaccagttctcgctcaagctcaacagcgtgaccgcggcggacaccgccgtgtactactgtgcccgggacggcggcatcgcagtcactgactactactattacggcctcgacgtgtggggccaggggacgacggtcacggtgagctccgcctccaccaaaggccccagcgtcttccccctcgcgccgtcctccaagtccacctcgggtggcaccgccgccctgggctgcctggtcaaggactacttcccggagcctgtgaccgtgtcctggaactcgggcgcgctcacgagcggcgtacacaccttcccggcggtgctccagtcctccgggctgtactcgctctcgtcggtcgtcacggtgccgtcctcctccctgggcacccagacctacatctgcaacgtgaaccacaagccgtccaacaccaaggtggataagaaggtcgagcccaagtcgtgcgacaagacgcacacgtgcccgccgtgcccggccccggagctgctgggcggcccctcggtcttcctgttccccccgaagcccaaggatacgctgatgatctcccgcaccccggaggtcacctgcgtggtggtggacgtctcccacgaggacccggaggtgaaattcaactggtacgtcgacggagtggaggtccacaacgccaagaccaagccccgggaggagcagtacaactccacgtaccgcgtcgtctccgtcctgaccgtcctccaccaggactggctgaacggcaaggagtacaagtgtaaggtctccaacaaggcgctgcccgcccccatcgagaagaccatctccaaggcaaagggtcagccgcgggagccgcaggtctataccctccccccgtcccgcgacgagctgacgaaaaaccaggtctccctgacctgcctggtgaagggtttctacccctccgacatcgcggtcgagtgggagtcgaacggccagccggagaacaactacaagaccaccccccccgtgctcgacagtgacggctcgttcttcctgtactcgaagctgaccgtcgacaagtcgcgctggcagcagggcaacgtcttctcgtgctccgttatgcacgaggccctgcacaaccactacacgcagaagagtctttcgctgtccccggggaagtgataatctagagtcggggcggccggcc

SEQ ID NO: 26 - DNA, 1456 bp, Artificial Sequence
  Synthetic 2.3D11 heavy chain with human IgG4
aagcttaccgccaccatggggtggtcgtgcatcatcctcttcctggtcgccaccgcgaccggcgtgcattcgcaggtccagctccaggagagcggcccgggcctggtgaagccctccggcacgctctctctgacgtgcgccgtctcgggagtgagtatccgctcgatcaactggtggaactgggtgcggcagccgccgggcaagggcctggaatggatcggggagatctaccactccgggtcgaccaactacaacccgagcctgaagtcccgggtcacgatcagcgtggacaagtccaagaaccagttctccctgaagctgaacagtgtaacggcggcggacacggcggtctactactgtgcgcgcgacggcggcatcgccgtgaccgattactactactacggcctcgacgtatggggccagggcaccaccgtcacggtgtcgagcgcatcgacgaagggcccctccgtgttccccctagccccgtgctcccgcagcacctctgagtccacggcggccttgggctgcctcgtgaaggactacttcccggagccggtcactgtgtcgtggaactccggcgcgctgaccagcggggtccacaccttccccgccgtcctgcagtcgtcgggcctgtactccctgagctcggtggtgaccgtcccctccagctccctcggcactaagacctatacctgcaacgtcgaccacaagccgtccaacaccaaggtggacaagcgagtggaatcgaagtacggcccgccctgcccctcctgccccgcccccgagttcctggggggcccgagcgtcttcctgttcccgccgaagccgaaggacacgctgatgatcagccggacgccggaagtgacgtgcgtcgtcgtggacgtgtcccaggaagaccctgaggtgcagttcaactggtacgtggacggcgtggaggtgcacaacgcgaaaaccaagccgcgcgaggagcagttcaacagcacctaccgcgtcgtgagcgtcctgacggtgctgcaccaggactggctcaacggcaaggagtacaagtgcaaggtatccaacaagggactgccgtcgtccatcgagaagaccatctccaaggccaagggccagccccgggagccccaagtctacaccctccccccgtcgcaggaggagatgacgaagaaccaggtctccctgacctgtctcgtcaagggcttctacccctccgacatcgccgtcgagtgggagtccaacgggcagcccgagaacaactacaagaccaccccgccagtcctggacagtgacgggtcgttcttcctgtactcccgactcactgtggacaagagccgctggcaagaggggaacgtcttctcctgctcagtgatgcacgaggccctccacaaccactacacccaaaagtcgctgtccctgtccctcgggaaatgataatctagagtcggggcggccggcc

SEQ ID NO: 27 - DNA, 1456 bp, Artificial Sequence
  Synthetic 2.3D11 heavy chain with human IgG4 Ser228Pro and Leu235Glu
aagcttaccgccaccatggggtggtcgtgcatcatcctcttcctggtcgccaccgcgaccggcgtgcattcgcaggtccagctccaggagagcggcccgggcctggtgaagccctccggcacgctctctctgacgtgcgccgtctcgggagtgagtatccgctcgatcaactggtggaactgggtgcggcagccgccgggcaagggcctggaatggatcggggagatctaccactccgggtcgaccaactacaacccgagcctgaagtcccgggtcacgatcagcgtggacaagtccaagaaccagttctccctgaagctgaacagtgtaacggcggcggacacggcggtctactactgtgcgcgcgacggcggcatcgccgtgaccgattactactactacggcctcgacgtatggggccagggcaccaccgtcacggtgtcgagcgcatcgacgaagggcccctccgtgttccccctagccccgtgctcccgcagcacctctgagtccacggcggccttgggctgcctcgtgaaggactacttcccggagccggtcactgtgtcgtggaactccggcgcgctgaccagcggggtccacaccttccccgccgtcctgcagtcgtcgggcctgtactccctgagctcggtggtgaccgtcccctccagctccctcggcactaagacctatacctgcaacgtcgaccacaagccgtccaacaccaaggtggacaagcgagtggaatcgaagtacggcccgccctgccccccctgccccgcccccgagttcgagggcgggccgagcgtcttcctgttcccgccgaagccgaaggacacgctgatgatcagccggacgccggaagtgacgtgcgtcgtcgtggacgtgtcccaggaagaccctgaggtgcagttcaactggtacgtggacggcgtggaggtgcacaacgcgaaaaccaagccgcgcgaggagcagttcaacagcacctaccgcgtcgtgagcgtcctgacggtgctgcaccaggactggctcaacggcaaggagtacaagtgcaaggtatccaacaagggactgccgtcgtccatcgagaagaccatctccaaggccaagggccagccccgggagccccaagtctacaccctccccccgtcgcaggaggagatgacgaagaaccaggtctccctgacctgtctcgtcaagggcttctacccctccgacatcgccgtcgagtgggagtccaacgggcagcccgagaacaactacaagaccaccccgccagtcctggacagtgacgggtcgttcttcctgtactcccgactcactgtggacaagagccgctggcaagaggggaacgtcttctcctgctcagtgatgcacgaggccctccacaaccactacacccaaaagtcgctgtccctgtccctcgggaaatgataatctagagtcggggcggccggcc

SEQ ID NO: 28 - DNA, 736 bp, Artificial Sequence
  Synthetic 2.3D11 light chain with human kappa
aagcttaccgccaccatggggtggtcgtgcatcatcctcttcctggtggccaccgccacgggcgtccactccgagatcgtcctgacccagtcccccgccaccctctccctgtcgccgggcgagcgggccacgctgtcgtgccgggcgtccgagtcggtctcgtcgaacctcgcctggtatcagcagaagcccggccaggcgccgcgcctcctcatctacggcgccttcaatcgcgccacgggcatccccgcccggttctccggctccggatcggggaccgacttcaccctcaccatctcctcgctggagccggaggacttcgccgtctactactgccagcaacggtcggactggttcaccttcggaggcggcaccaaggtcgagatcaagacggtggccgcgccgagcgtcttcatcttcccgccttccgacgagcagctcaagtccgggaccgcctccgtagtatgcctcctcaataacttctacccccgggaggcgaaggtccagtggaaggtcgacaacgccctccaatcgggcaactcccaggagtcggtgaccgagcaggattccaaggactcgacctacagtctaagctccaccctcacactgtcgaaggcggactacgagaagcacaaggtgtacgcctgcgaggtcacccaccagggcctgagcagcccggtcaccaagtccttcaaccggggcgagtgctgataatctagagtcggggcggccggcc